(19) United States
(12) Patent Application Publication
(10) Pub. No.: US 2018/0016309 A1
(43) Pub. Date: Jan. 18, 2018

(54) NOVEL BT TOXIN RECEPTORS AND METHODS OF USE

(71) Applicants: PIONEER HI-BRED INTERNATIONAL, INC., JOHNSTON, IA (US); E. I. DU PONT DE NEMOURS AND COMPANY, WILMINGTON, DE (US)

(72) Inventors: JAMES E. BECKER, CLIVE, IA (US); CATHERINE J. FINKE, ALTOONA, IA (US); JOHN P. MATHIS, JOHNSTON, IA (US); MARK EDWARD NELSON, WAUKEE, IA (US)

(73) Assignees: PIONEER HI-BRED INTERNATIONAL, INC., JOHNSTON, IA (US); E. I. DU PONT DE NEMOURS AND COMPANY, WILMINGTON, DE (US)

(21) Appl. No.: 15/548,341

(22) PCT Filed: Jan. 20, 2016
(86) PCT No.: PCT/US2016/014008

§ 371 Date: Aug. 2, 2017

Related U.S. Application Data
(60) Provisional application No. 62/111,958, filed on Feb. 4, 2015.

(51) Int. Cl. C07K 14/435; G01N 33/68; A01K 67/033; C07K 16/28; C12N 15/113; C12N 15/82; C12Q 1/68; C07K 14/325

(52) U.S. Cl. CPC C07K 14/43563; C12Q 1/6876; G01N 33/6872; C07K 14/325; C07K 16/28; C12N 15/1138; C12N 15/8286; A01K 67/0333; A01K 2217/052; A01K 2267/01; C12N 2310/14; C12Q 2600/124; C12Q 2600/158; G01N 2333/325; G01N 2500/04; G01N 2333/43552

(57) ABSTRACT

The disclosure relates to Bt toxin resistance management. One embodiment relates to the isolation and characterization of polynucleotides and polypeptides corresponding to novel Bt toxin receptors. The polynucleotides and polypeptides are useful in identifying or designing novel Bt toxin receptor ligands including novel insecticidal toxins.

FIELD

This disclosure is directed to the manipulation of Bt toxin susceptibility in plant pests. One embodiment relates to the isolation and characterization of nucleic acids and polypeptides for novel Bt toxin receptors. The nucleic acids and polypeptides are useful in improving insecticides, developing new insecticides, and monitoring insect resistance.

### BACKGROUND

Insect pests are a major factor in the loss of the world's agricultural crops. For example, armyworm feeding, black cutworm damage, or European corn borer damage can be economically devastating to agricultural producers. Insect pest-related crop loss from attacks on field and sweet corn alone has reached about one billion dollars a year in damage and control expenses.

Traditionally, growers have used chemical pesticides as a means to control agronomically important pests. The introduction of transgenic plants carrying the delta-endotoxin from Bacillus thuringiensis (Bt) afforded a non-chemical method of control. Bt toxins have traditionally been categorized by their specific toxicity towards specific insect categories. For example, the Cry1 group of toxins are toxic to Lepidoptera. The Cry1 group includes, but is not limited to, Cry1Aa, Cry1Ab and Cry1Ac. See Hofte et al (1989) Microbiol Rev 53: 242-255.

Lepidopteran insects cause considerable damage to maize crops throughout North America and the world. One of the leading pests is Ostrinia nubulalis, commonly called the European corn borer (ECB). Genes encoding the crystal proteins Cry1Ab and Cry1Ac from Bt have been introduced into maize as a means of ECB control as well as other pests. These transgenic maize hybrids have been effective in control of ECB. However, developed resistance to Bt toxins presents a challenge in pest control. See McGaughey et al. (1998) Nature Biotechnology 16: 144-146; Estruch et al. (1997) Nature Biotechnology 15:137-141; Roush et al. (1997) Nature Biotechnology 15 816-817; and Hofte et al. (1989) Microbiol. Rev. 53: 242-255.

A primary site of action of Cry1 toxins is in the brush border membranes of the midgut epithelia of susceptible insect larvae such as lepidopteran insects. Cry1A toxin binding polypeptides have been characterized from a variety of Lepidopteran species. A Cry1A(c) binding polypeptide with homology to an aminopeptidase N has been reported from Manduca sexta, Lymantria dispar, Helicoverpa zea and Heliothis virescens. See Knight et al (1994) Mol Micro 11: 429-436; Lee et al. (1996) Appl Environ Micro 63: 2845-2849; Gill et al. (1995) J Biol. Chem 270: 27277-27282; and Garczynski et al. (1991) Appl Environ Microbiol 10: 2816-2820.

Another Bt toxin binding polypeptide (BTR1) cloned from M sexta has homology to the cadherin polypeptide superfamily and binds Cry1A(a), Cry1A(b) and Cry1A(c). See Vadlamudi et al. (1995) J Biol Chem 270(10):5490-4, Keeton et al. (1998) Appl Environ Microbiol 64(6):2158-2165; Keeton et al. (1997) Appl Environ Microbiol 63(9):3419-3425 and U.S. Pat. No. 5,693,491.

A homologue of BTR1 that demonstrates binding to Cry1A(a) was isolated from Bombyx mori as described in Ihara et al. (1998) Comparative Biochemistry and Physiology, Part B 120:197-204 and Nagamatsu et al. (1998) Biosci. Biotechnol. Biochem. 62(4):727-734. In addition, a Bt-binding protein that is also a member of the cadherin superfamily was isolated from Heliothis virescens, the tobacco budworm. See Gahan et al. (2001) Science 293:857-860 and GenBank accession number AF367362.

Similarly, the Cry2 class of Bt toxins are toxic to lepidopteran insects, and specifically, Helicoverpa zea. Cry2Ab specifically binds to H. zea midgut tissue to a binding site similar to other Cry2A family toxins, but different from that of Cry1Ac toxins. See Hernandez-Rodriguez et al (2008) Appl Environ Microbiol 74(24): 7654-7659. A specific receptor for Cry2A class toxins has yet to be identified. Furthermore, binding site alteration of a receptor has been proposed as a mechanism of resistance to Cry2A class toxins. See Caccia et al (2010) Plos One 5(4):e9975.

Identification of the plant pest binding polypeptides for Bt toxins are useful for investigating Bt toxin-Bt toxin receptor interactions, selecting and designing improved toxins or other insecticides, developing novel insecticides, and screening for resistance or other resistance management strategies and tools.

### BRIEF SUMMARY

Compositions and methods for modulating susceptibility of a cell to Bt toxins are provided. The compositions include Bt toxin receptor polypeptides and fragments and variants thereof, from the lepidopteran insects corn earworm (CEW, Helicoverpa zea) and European corn borer (ECB, Ostrinia nubilalis), fall armyworm (FAW, Spodoptera frugiperda), and soybean looper (SBL, Chrysodeixis includens). Nucleic acids encoding the polypeptides, antibodies specific to the polypeptides, and nucleic acid constructs for expressing the polypeptides in cells of interest are also provided.

The methods provided here are useful for investigating the structure-function relationships of Bt toxin receptors; investigating toxin-receptor interactions; elucidating the mode of action of Bt toxins; screening and identifying novel Bt toxin receptor ligands including novel insecticidal toxins; designing and developing novel Bt toxin receptor ligands; and creating insects or insect colonies with altered susceptibility to insecticidal toxins.

The methods provided here are also useful for managing Bt toxin resistance in plant pests, for monitoring of toxin resistance in plant pests, and for protecting plants against damage by plant pests.

### BRIEF DESCRIPTION OF THE DRAWINGS

FIG. 1A: An in-solution competitive binding assay was performed using 40 μg of midgut derived brush border membrane vesicles (BBMVs) from Helicoverpa zea (corn earworm) and 10 nM IP2.127 labeled with Alexa Fluor®-488 (hereinafter Alexa-488 or Alexa; Life Technologies Invitrogen) fluorescence molecule (Alexa IP2.127) that had been enzymatically derived from full length IP2.127 by treatment with purified trypsin to simulate host midgut processing. Binding buffer used for IP2.127 binding was a sodium carbonate buffer consisting of 50 mM sodium carbonate/HCl pH 9.6, 150 mM NaCl, 0.1% Tween 20. Total binding sample (“Total” on graph) contained 40 μg of BBMVs from Helicoverpa zea (corn earworm) and 10 nM Alexa IP2.127 in binding buffer. Nonspecific binding sample (“Nonspecific” on graph) contained same as Total binding sample with the addition of 1 μM IP2.127 and reflects non-receptor mediated interaction of labeled IP2.127. Samples were incubated at room temperature and then unbound IP2.127 was separated by centrifugation allowing IP2.127 bound to BBMVs to be subjected to SDS-PAGE. Binding signal was monitored by in-gel fluorescence using a laser scanner and quantified by densitometry. The difference between the binding signal measured for the “Nonspecific” sample and the signal measured for the “Total” sample represents the specific interaction of Alexa-IP2.127 with its receptor(s) in H. zea BBMVs.

FIG. 1B: An in-solution competitive binding assay was performed using 40 μg of midgut derived brush border membrane vesicles (BBMVs) from Ostrinia nubilalis (European corn borer) and 10 nM IP2.127 labeled with Alexa-488 fluorescence molecule (Alexa IP2.127) that had been enzymatically derived from full length IP2.127 by treatment with purified trypsin to simulate host midgut processing. Binding buffer used for IP2.127 binding was a sodium carbonate buffer consisting of 50 mM sodium carbonate/HCl pH 9.6, 150 mM NaCl, 0.1% Tween 20. Total binding sample (“Total” on graph) contained 40 μg of BBMVs from Ostrinia nubilalis (European corn borer) and 10 nM Alexa IP2.127 in binding buffer. Nonspecific binding sample (“Nonspecific” on graph) contained same as Total binding sample with the addition of 1 μM IP2.127 and reflects non-receptor mediated interaction of labeled IP2.127. Samples were incubated at room temperature and then unbound IP2.127 was separated by centrifugation allowing IP2.127 bound to BBMVs to be subjected to SDS-PAGE. Binding signal was monitored by in-gel fluorescence using a laser scanner and quantified by densitometry. The difference between the binding signal measured for the “Nonspecific” sample and the signal measured for the “Total” sample represents the specific interaction of Alexa-IP2.127 with its receptor(s) in O. nubilalis BBMVs.

FIG. 1C: An in-solution competitive binding assay was performed using 20 μg of midgut derived brush border membrane vesicles (BBMVs) from Spodoptera frugiperda (Fall Armyworm) and 10 nM IP2.127 labeled with Alexa-488 fluorescence molecule (Alexa IP2.127) that had been enzymatically derived from full length IP2.127 by treatment with purified trypsin to simulate host midgut processing. Binding buffer used for IP2.127 binding was a sodium carbonate buffer consisting of 50 mM sodium carbonate/HCl pH 9.6, 150 mM NaCl, 0.1% Tween 20. Total binding sample (“Total” on graph) contained 20 μg of BBMVs from Spodoptera frugiperda (Fall Armyworm) and 10 nM Alexa IP2.127 in binding buffer. Nonspecific binding sample (“Nonspecific” on graph) contained same as Total binding sample with the addition of 1 μM IP2.127 and reflects non-receptor mediated interaction of labeled IP2.127. Samples were incubated at room temperature and then unbound IP2.127 was separated by centrifugation allowing IP2.127 bound to BBMVs to be subjected to SDS-PAGE. Binding signal was monitored by in-gel fluorescence using a laser scanner and quantified by densitometry.

FIG. 1D: An in-solution competitive binding assay was performed using 40 μg of midgut derived brush border membrane vesicles (BBMVs) from Chrysodeixis includens (Soybean Looper) and 5 nM IP2.127 labeled with Alexa-488 fluorescence molecule (Alexa IP2.127) that had been enzymatically derived from full length IP2.127 by treatment with purified trypsin to simulate host midgut processing. Binding buffer used for IP2.127 binding was a CAPS buffer consisting of 20 mM CAPS, 150 mM NaCl, 0.1% Tween 20, pH 10.5. Total binding sample (“Total” on graph) contained 40 μg of BBMVs from Chrysodeixis includens (Soybean Looper) and 5nM Alexa IP2.127 in binding buffer. Nonspecific binding sample (“Nonspecific” on graph) contained same as Total binding sample with the addition of 1 μM IP2.127 and reflects non-receptor mediated interaction of labeled IP2.127. Samples were incubated at room temperature and then unbound IP2.127 was separated by centrifugation allowing IP2.127 bound to BBMVs to be subjected to SDS-PAGE. Binding signal was monitored by in-gel fluorescence using a laser scanner and quantified by densitometry.

FIG. 2A: Binding assay/co-precipitation sample compositions are: lane 1, Binding buffer; lane 2, Molecular weights standards; lane 3, 100 nM biotin-labeled IP2.127 and 1 μM IP2.127; lane 4, 100 nM biotin-labeled IP2.127; lane 5, 1 μM IP2.127; lane 6, 500 μg H. zea BBMVs; lane 7, 1 μM biotin-labeled IP2.127 and 500 μg H. zea BBMVs; lane 8, 100 nM biotin-labeled IP2.127 and 500 μg H. zea BBMVs; Note the unique band in lanes 7 and 8 (indicated by the arrow) that is absent from lane 6 (BBMVs in the absence of biotin-labeled IP2.127). The unique band was extracted from the gel and further analyzed.

FIG. 2B: Binding assay/co-immunoprecipitation sample compositions are: lanes 1 and 8, Molecular weights standards; lane 2, binding buffer; lane 3, 1 μM IP2.127; lane 4, 500 μg O. nubilalis BBMVs; lane 5, 1 μM IP2.127 and 500 μg O. nubilalis BBMVs with no antibody; lane 6, 1 μM IP2.127 and 500 μg O. nubilalis BBMVs; lane 7, 100 nM IP2.127 and 500μg O. nubilalis BBMVs; lane 9, 100 nM IP2.127 used as gel standard (assay/co-immunoprecipitation sample.) Note the unique band with the arrow in lane 6 that is also present in lane 7, but at lower intensity consistent with the lower concentration of IP2.127. The unique band was extracted from the gel and further analyzed.

FIG. 2C: Binding assay/co-immunoprecipitation sample compositions are: lanes 1 and 8, Molecular weights standards; lane 2, Binding buffer; lane 3, 1 μM IP2.127; lane 4, 500 μg S. frupperda BBMVs; lane 5, 1 μM IP2.127 and 500 μg S. frupperda BBMVs with no antibody; lane 6, 1 μM IP2.127 and 500 μg S. frupperda BBMVs; lane 7, 100 nM IP2.127 and 500 μg S. frupperda BBMVs; lane 9, 100 nM IP2.127 used as gel standard (assay/co-immunoprecipitation sample). The band indicated by the arrow was extracted from the gel and further analyzed.

FIG. 2D: Binding assay/co-immunoprecipitation sample compositions are lane 1 and 8, Molecular weights standards; lane 2, Binding buffer; lane 3, 1 μM IP2.127; lane 4, 500μg C. includens BBMVs; lane 5, 1 μM IP2.127 and 500 μg C. includens BBMVs with no antibody; lane 6, 1 μM IP2.127 and 500 μg C. includens BBMVs; lane 7, 100 nM IP2.127 and 500 μg C. includens BBMVs; lane 9, 100 nM IP2.127 used as gel standard (assay/co-immunoprecipitation sample). The band indicated by the arrow was extracted from the gel and further analyzed.

FIG. 3A: FIG. 3A represents the peptide sequences of SEQ ID NO: 2 from the protein band identified by mass spectrometry. Peptides identified by mass spectrometry are in bold.

FIG. 3B: FIG. 3B represents the peptide sequences of SEQ ID NO: 4 from the protein band identified by mass spectrometry. Peptides identified by mass spectrometry are in bold.

FIG. 3C: FIG. 3C represents the peptide sequences of SEQ ID NO: 8 from the protein band identified by mass spectrometry. Peptides identified by mass spectrometry are in bold.

FIG. 3D: FIG. 3D represents the peptide sequences of SEQ ID NO: 10 from the protein band identified by mass spectrometry. Peptides identified by mass spectrometry are in bold.

FIG. 4: A depiction of SEQ ID NO: 2 with the diamonds representing transmembrane regions and dashes representing the peptide sequences identified by mass spectrometry. Transmembrane region 1 is defined from about amino acid 22 to about amino acid 65; transmembrane region 2 is defined from about amino acid 332 to about amino acid 375; transmembrane region 3 is defined from about amino acid 375 to about amino acid 418; transmembrane region 4 is defined from about amino acid 407 to about amino acid 447; transmembrane region 5 is defined from about amino acid 479 to about amino acid 522; transmembrane region 6 is defined from about amino acid 1116 to about amino acid 1139; transmembrane region 7 is defined from about amino acid 1158 to about amino acid 1201; transmembrane region 8 is defined from about amino acid 1195 to about amino acid 1238; transmembrane region 9 is defined from about amino acid 1234 to about amino acid 1267; transmembrane region 10 is defined from about amino acid 1261 to about amino acid 1304; and transmembrane region 11 is defined from about amino acid 1334 to about amino acid 1377.

### DETAILED DESCRIPTION

The embodiments provided herein are directed to novel receptor polypeptides having Bt toxin binding activity, the receptors being derived from the order Lepidoptera. Receptor polypeptides disclosed herein are derived from the superfamilies including the Noctuidae, particularly from Helicoverpa zea, Spodoptera frugiperda, and Chrysodeixis includens, and the Crambidae, particularly from Ostrinia nubilalis and have Bt binding activity. The polypeptides have homology to members of the ABC Transporter family of proteins, more specifically, to members of the ABC Transporter subfamilies A and G.

Accordingly, one embodiment provides for isolated nucleic acid molecules comprising nucleotide sequences encoding polypeptides having Bt toxin binding activity shown in SEQ ID NO: 2, 4, 6, 8, 10 or 12; or the respective encoding polynucleotide sequences of SEQ ID NO: 1, 3, 5, 7, 9 or 11. Further provided are fragments and variant polypeptides described herein.

The term “nucleic acid” refers to all forms of DNA such as cDNA and RNA such as mRNA, as well as analogs of the DNA or RNA generated using nucleotide analogs. The nucleic acid molecules can be single stranded or double stranded. Strands can include the coding or non-coding strand.

One embodiment encompasses isolated or substantially purified nucleic acids or polypeptide compositions. An “isolated” or “purified” nucleic acid molecule or polypeptide, or biologically active portion thereof, is substantially free of other cellular material, or culture medium when produced by recombinant techniques, or substantially free of chemical precursors or other chemicals when chemically synthesized. An “isolated” nucleic acid can be free of sequences (preferably polypeptide encoding sequences) that naturally flank the nucleic acid (i.e., sequences located at the 5′ and 3′ ends of the nucleic acid) in the genomic DNA of the organism from which the nucleic acid is derived. For example, in one embodiment, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. One embodiment contemplates polypeptide that is substantially free of cellular material including preparations of polypeptide having less than about 30%, 20%, 10%, 5%, (by dry weight) of contaminating polypeptide. When the polypeptide or biologically active portion thereof is recombinantly produced, the culture medium may represent less than about 30%, 20%, 10%, or 5% (by dry weight) of chemical precursors or non-polypeptide-of-interest chemicals.

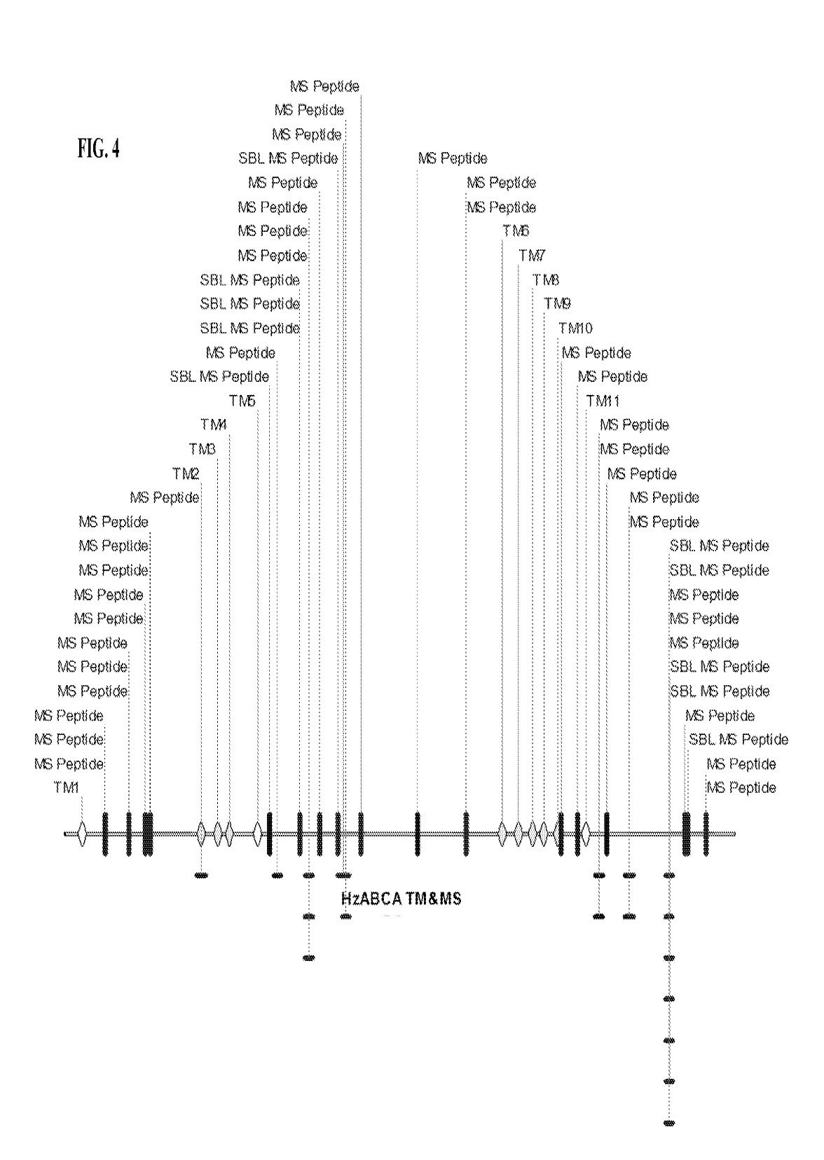

In another embodiment, polypeptide preparations may contain contaminating material that does not interfere with the specific desired activity of the polypeptide. The compositions also encompass fragments and variants of the disclosed nucleotide sequences and the polypeptides encoded thereby. In one embodiment, a fragment comprises a transmembrane fragment (FIG. 4).

Polynucleotide compositions are useful for, among other uses, expressing the receptor polypeptides in cells of interest to produce cellular or isolated preparations of said polypeptides for investigating the structure-function and/or sequence-function relationships of Bt toxin receptors, evaluating toxin-receptor interactions, elucidating the mode of action of Bt toxins, screening test compounds to identify novel Bt toxin receptor ligands including novel insecticidal toxins, and designing and developing novel Bt toxin receptor ligands including novel insecticidal toxins.

The isolated polynucleotides encoding the receptor polypeptides of the embodiment may be expressed in a cell of interest; and the Bt toxin receptor polypeptides produced may be utilized in intact cell or in-vitro receptor binding assays, and/or intact cell toxicity assays. Methods and conditions for Bt toxin binding and toxicity assays are known in the art and include but are not limited to those described in U.S. Pat. No. 5,693,491; T. P. Keeton et al. (1998) Appl. Environ. Microbiol. 64(6):2158-2165; B. R. Francis et al. (1997) Insect Biochem. Mol. Biol. 27(6):541-550; T. P. Keeton et al. (1997) Appl. Environ. Microbiol. 63(9):3419-3425; R. K. Vadlamudi et al. (1995) J. Biol. Chem. 270(10):5490-5494; Ihara et al. (1998) Comparative Biochem. Physiol. B 120:197-204; and Nagamatsu et al. (1998) Biosci. Biotechnol. Biochem. 62(4):727-734.

As used herein, a “Bt toxin” refers to genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC® Accession Numbers 40098, 67136, 31995 and 31998. Members of these classes of B. thuringiensis insecticidal proteins include, but are not limited to, Cry proteins well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix).

By “cell of interest” is intended any cell in which expression of the polypeptides disclosed herein is desired. Cells of interest include, but are not limited to mammalian, avian, insect, plant, bacteria, fungi and yeast cells. Cells of interest include but are not limited to cultured cell lines, primary cell cultures, cells in vivo, and cells of transgenic organisms.

As used herein, a “modified” or “altered” sequence refers to a sequence that differs from the wildtype sequence. In one embodiment, a modified or altered polynucleotide sequence differs from SEQ ID NOs: 1, 3, 5, 7, 9, 11, or 13-15. In another embodiment, a modified or altered amino acid sequence differs from SEQ ID NO: 2, 4, 6, 8, 10 or 12. In one embodiment, a modification or alteration in a sequence can be screened to determine an altered susceptibility to a Bt toxin. The methods embodied contemplate the use of polypeptides and polynucleotides disclosed herein in receptor binding and/or toxicity assays to screen test compounds to identify novel Bt toxin receptor ligands, including receptor agonists and antagonists, or to screen for resistance. Test compounds include molecules available from diverse libraries of small molecules created by combinatorial synthetic methods. Test compounds also include, but are not limited to, antibodies, binding peptides, and other small molecules designed or deduced to interact with the receptor polypeptides of the embodiment. Test compounds may also include peptide fragments of the receptor, anti-receptor antibodies, anti-idiotypic antibodies mimicking one or more receptor binding domains of a toxin, binding peptides, chimeric peptides, and fusion, or heterologous polypeptides, produced by combining two or more toxins or fragments thereof, such as extracellular portions of the receptors disclosed herein and the like. Ligands identified by the screening methods of the embodiment include potential novel insecticidal toxins, the insecticidal activity of which can be determined by known methods; for example, as described in U.S. Pat. Nos. 5,407,454, 5,986,177, and 6,232,439.

In one embodiment, the methods relate to isolating receptors of insect midgut toxins comprising dissecting an insect midgut tissue; performing a membrane enrichment step on the insect midgut tissue, such as a BBMV preparation; performing an in-solution binding assay on the enriched membrane with an insect toxin; and performing an affinity purification, wherein the toxin is the affinity purification target. In another embodiment, performing a membrane enrichment step may be performed on a whole insect. In another embodiment, the affinity purification may be performed prior to the in-solution binding step. In one embodiment, the affinity purification target is the insect toxin. In another embodiment, the affinity purification target is the receptor polypeptide.

The embodiment provides methods for screening ligands that bind to the polypeptides disclosed herein. Both the polypeptides and fragments thereof (for example, toxin binding peptides) may be used in screening assays for compounds that bind to receptor peptides and exhibit desired binding characteristics. Desired binding characteristics include, but are not limited to binding affinity, binding site specificity, association and dissociation rates, and the like. The screening assays may be conducted in intact cells or in in vitro assays which include exposing a ligand binding domain to a sample ligand and detecting the formation of a ligand-binding polypeptide complex. The assays may be direct ligand-receptor binding assays, ligand competition assays, or indirect assays designed to measure impact of binding on transporter function, for example, ATP hydrolysis, conformational change, or solute transport.

The methods comprise providing at least one Bt toxin receptor polypeptide disclosed herein, contacting the polypeptide with a sample and a control ligand under conditions promoting binding, and determining binding characteristics of sample ligands, relative to control ligands. Methods for conducting a binding assay are known in the art. For in vitro binding assays, the polypeptide may be provided as isolated, lysed, or homogenized cellular preparations. Isolated polypeptides may be provided in solution, or immobilized to a matrix. Methods for immobilizing polypeptides are well known in the art, and include but are not limited to construction and use of fusion polypeptides with commercially available high affinity ligands. For example, GST fusion proteins can be adsorbed onto glutathione sepharose beads (Sigma Chemical, St. Louis, Mo.) or glutathione derivatized microtitre plates. The polypeptides may also be immobilized using biotin and streptavidin, or chemical conjugation (linking) of polypeptides to a matrix through techniques known in the art. Alternatively, the polypeptides may be provided in intact cell binding assays in which the polypeptides are generally expressed as cell surface Bt toxin receptors.

The disclosure provides methods utilizing intact cell toxicity assays to screen for ligands that bind to the receptor polypeptides disclosed herein and confer toxicity upon a cell of interest expressing the polypeptide in the presence of a Bt toxin. A ligand selected by this screening is a potential insecticidal toxin to insects expressing the receptor polypeptides, particularly enterally. The insect specificity of a particular Bt toxin may be determined by the presence of the receptor in specific insect species. Binding of the toxins may be specific for the receptor of some insect species and while insignificant or nonspecific for other variant receptors. See, for example Hofte et al. (1989) Microbiol Rev 53: 242-255. The toxicity assays include exposing, in intact cells expressing a polypeptide of the embodiment, the toxin binding domain of a polypeptide to a sample ligand and detecting the toxicity effected in the cell expressing the polypeptide. By “toxicity” is intended the decreased viability of a cell. By “viability” is intended the ability of a cell to proliferate and/or differentiate and/or maintain its biological characteristics in a manner characteristic of that cell in the absence of a particular cytotoxic agent.

In one embodiment, the methods comprise providing at least one cell surface Bt toxin receptor polypeptide comprising SEQ ID NO: 2, 4, 6, 8, 10, 12 or an extracellular toxin binding domain thereof, contacting the receptor polypeptide with a sample and a control ligand under conditions promoting binding, and determining the viability of the cell expressing the cell surface Bt toxin receptor polypeptide, relative to the control ligand.

By “contacting” is intended that the sample and control agents are presented to the intended ligand binding site of the polypeptides of the embodiment.

By “conditions promoting binding” is intended any combination of physical and biochemical conditions that enables a ligand of the polypeptides of the embodiment to bind the intended polypeptide over background levels. Examples of such conditions for binding of Cry2 toxins to Bt toxin receptors, as well as methods for assessing the binding, are known in the art and include but are not limited to those described in Keeton et al. (1998) Appl Environ Microbiol 64(6): 2158-2165; Francis et al. (1997) Insect Biochem Mol Biol 27(6):541-550; Keeton et al. (1997) Appl Environ Microbiol 63(9):3419-3425; Vadlamudi et al. (1995) J Biol Chem 270(10):5490-5494; Ihara et al. (1998) Comparative Biochemistry and Physiology, Part B 120:197-204; and Nagamatsu et al. (1998) Biosci. Biotechnol. Biochem. 62(4):727-734. In this aspect, commercially available methods for studying protein-protein interactions, such as yeast and/or bacterial two-hybrid systems could also be used. Two-hybrid systems are available from, for example, Clontech (Palo Alto, Calif.) or Display Systems Biotech Inc. (Vista, Calif.).

The compositions and screening methods disclosed herein are useful for designing and developing novel Bt toxin receptor ligands including novel insecticidal toxins. Various candidate ligands; ligands screened and characterized for binding, toxicity, and species specificity; and/or ligands having known characteristics and specificities may be linked or modified to produce novel ligands having particularly desired characteristics and specificities. The methods described herein for assessing binding, toxicity and insecticidal activity may be used to screen and characterize the novel ligands.

The compositions and screening methods disclosed herein are useful for designing and developing novel Bt toxin receptor-ligand complexes, wherein both the receptor and ligand are expressed in the same cell. By “complexes” is intended that the association of the receptor to the ligand is sufficient to prevent other interactions to the ligand in the cell. The receptor may be receptors described herein, or variants or fragments thereof. Also, the receptor may be a heterologous polypeptide, retaining biological activity of the receptor polypeptides described herein.

In one embodiment, the sequences encoding the receptors, and variants and fragments thereof, are used with yeast and bacterial two-hybrid systems to screen for Bt toxins of interest (for example, more specific and/or more potent toxins), or for insect molecules that bind the receptor and can be used in developing novel insecticides.

By “linked” is intended that a covalent bond is produced between two or more molecules. Methods that may be used for modification and/or linking of polypeptide ligands such as toxins, include mutagenic and recombinogenic approaches including, but not limited to, site-directed mutagenesis, chimeric polypeptide construction, and DNA shuffling. Polypeptide modification methods also include methods for covalent modification of polypeptides. “Operably linked” means that the linked molecules carry out the function intended by the linkage.

The compositions and screening methods are useful for targeting ligands to cells expressing the receptor polypeptides. For targeting, secondary polypeptides, and/or small molecules which do not bind the receptor polypeptides are linked with one or more primary ligands which bind the receptor polypeptides disclosed herein, including but not limited to a Cry2A toxin, and more particularly an IP2.127 toxin (SEQ ID NO: 20 and 21), a variant, or a fragment thereof. (See SEQ ID NOs: 133 and 134 of U.S. Pat. No. 7,208,474). By linkage, any polypeptide and/or small molecule linked to a primary ligand may be targeted to the receptor polypeptide, and thereby to a cell expressing the receptor polypeptide; wherein the ligand binding site is available at the extracellular surface of the cell.

In one embodiment, at least one secondary polypeptide toxin is linked with a primary Cry2A toxin capable of binding the receptor polypeptides of SEQ ID NO: 2, 4, 6, 8, 10, or 12 to produce a toxin that is targeted and toxic to insects expressing the receptor for the primary toxin. Such insects include those of the order Lepidoptera, superfamilies including the Noctuidae and particularly from Helicoverpa zea, Spodoptera frugiperda, and Chrysodeixis includens, and the Crambidae and particularly from Ostrinia nubilalis. Such a combination toxin is particularly useful for eradicating or reducing crop damage by insects that have developed resistance to the primary toxin.

For expression of the Bt toxin receptor polypeptides of SEQ ID NO: 2, 4, 6, 8, 10, or 12, variants, or fragments in a cell of interest, the Bt toxin receptor sequences may be provided in expression cassettes. The cassette may include 5′ and 3′ regulatory sequences operably linked to a Bt toxin receptor sequence. In this aspect, by “operably linked” is intended a functional linkage between a promoter and a second sequence, wherein the promoter sequence initiates and mediates transcription of the DNA sequence corresponding to the second sequence. In reference to nucleic acids, generally, operably linked means that the nucleic acid sequences being linked are contiguous and, where necessary to join two polypeptide coding regions, contiguous and in the same reading frame. The cassette may additionally contain at least one additional gene to be cotransformed into the organism. Alternatively, the additional gene(s) may be provided on multiple expression cassettes.

Such an expression cassette may be provided with a plurality of restriction sites for insertion of the Bt toxin receptor sequence to be under the transcriptional regulation of the regulatory regions. The expression cassette may additionally contain selectable marker genes.

The expression cassette may include in the 5′-3′ direction of transcription, a transcriptional and translational initiation region (i.e., a promoter), a Bt toxin receptor nucleotide sequence, and a transcriptional and translational termination region (i.e., termination region) functional in host cells. The transcriptional initiation region, the promoter, may be native or analogous, or foreign or heterologous to the plant host and/or to the Bt toxin receptor sequence. Additionally, the promoter may be the natural sequence or alternatively a synthetic sequence. Where the promoter is “foreign” or “heterologous” to the plant host, is intended that the promoter is not found in the native host cells into which the promoter is introduced. Where the promoter is “foreign” or “heterologous” to the Bt toxin receptor sequence, it is intended that the promoter is not the native or naturally occurring promoter for the operably linked Bt toxin receptor sequence.

Heterologous promoters or native promoter sequences may be used in construct design. Such constructs may change expression levels of a Bt toxin receptor in a cell of interest, resulting in alteration of the phenotype of the cell.

The termination region may be native with the transcriptional initiation region, may be native with the operably linked DNA sequence of interest, may be native with the plant host, or may be derived from another source (i.e., foreign or heterologous to the promoter, the Bt toxin receptor sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, a gene may be optimized for increased expression in a particular transformed cell of interest. That is, the genes may be synthesized using host cell-preferred codons for improved expression.

Additional sequence modifications may enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (encephalomyocarditis 5′ noncoding region; Elroy-Stein et al. (1989) PNAS USA 86:6126-6130); potyvirus leaders, for example, TEV leader (tobacco etch virus; Allison et al. (1986); MDMV leader (maize dwarf mosaic virus), and human immunoglobulin heavy-chain binding polypeptide (BiP), (Macejak et al. (1991) Nature 353:90-94); untranslated leader from the coat polypeptide mRNA of alfalfa mosaic virus (AMV RNA 4); Jobling et al. (1987) Nature 325:622-625); tobacco mosaic virus leader (TMV; Gallie et al. (1989) in Molecular Biology of RNA, ed. Cech (Liss, New York), pp. 237-256); and maize chlorotic mottle virus leader (MCMV; Lommel et al. (1991) Virology 81:382-385). See also, Della-Cioppa et al. (1987) Plant Physiol. 84:965-968. Other methods to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Using the nucleic acids disclosed herein, the polypeptides may be expressed in any cell of interest, the particular choice of the cell depending on factors such as the level of expression and/or receptor activity desired. Cells of interest include, but are not limited to mammalian, plant, insect, bacteria, and yeast host cells. The choice of promoter, terminator, and other expression vector components will also depend on the cell chosen. The cells produce the protein in a non-natural condition (e.g., in quantity, composition, location, and/or time), because they have been genetically altered through human intervention to do so.

Those of skill in the art are knowledgeable in the numerous expression systems available for expression of a nucleic acid encoding a protein of the present embodiment. In brief summary, the expression of isolated nucleic acids encoding a protein of the present embodiment will typically be achieved by operably linking, for example, the DNA or cDNA to a promoter, followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present embodiment. To obtain high level expression of a cloned gene, it is desirable to construct expression vectors which contain, at the minimum, a strong promoter to direct transcription, a ribosome binding site for translational initiation, and a transcription or translation terminator. One of skill would recognize that modifications can be made to a protein of the present embodiment without diminishing its biological activity. Some modifications may be made to facilitate the cloning, expression, or incorporation of the targeting molecule into a heterologous polypeptide. Such modifications include, for example, a methionine added at the amino terminus to provide an initiation site, or additional amino acids (e.g., poly His) placed on either terminus to create conveniently located restriction sites or termination codons or purification sequences.

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda-derived P L promoter and N-gene ribosome binding site (Shimatake et al. (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present embodiment are available using Bacillus sp. and Salmonella. See, Palva et al. (1983) Gene 22:229-235 and Mosbach et al. (1983) Nature 302:543-545.

A variety of eukaryotic expression systems such as yeast, insect cell lines, plant and mammalian cells, are known to those of skill in the art. The sequences disclosed herein may be expressed in these eukaryotic systems. In some embodiments, transformed/transfected plant cells are employed as expression systems for production of the receptor proteins.

Synthesis of heterologous proteins in yeast is well known. See, for example, Sherman, F. et al. (1982) Methods in Yeast Genetics, Cold Spring Harbor Laboratory, which describes the various methods available to produce the protein in yeast. Two widely utilized yeast for production of eukaryotic proteins are Saccharomyces cerevisia and Pichia pastoris. Vectors, strains, and protocols for expression in Saccharomyces and Pichia are known in the art and available from commercial suppliers (e.g., Invitrogen Life Technologies, Carlsbad, Calif.). Suitable vectors usually have expression control sequences, such as promoters, for example 3-phosphoglycerate kinase or alcohol oxidase, and an origin of replication, termination sequences and the like as desired.

Polypeptides disclosed herein, once expressed, may be isolated from yeast by lysing the cells and applying standard protein isolation techniques to the lysates. The monitoring of the purification process may be accomplished by using Western blot techniques or radioimmunoassay or other standard immunoassay techniques.

The sequences encoding polypeptides disclosed herein may also be ligated to various expression vectors for use in transfecting cell cultures of, for instance, mammalian, insect, or plant origin. Illustrative of cell cultures useful for the production of the peptides are mammalian cells. Mammalian cell systems often will be in the form of monolayers of cells although mammalian cell suspensions may also be used. A number of suitable host cell lines capable of expressing intact proteins have been developed in the art, and include the COS, HEK293, BHK21, and CHO cell lines. Expression vectors for these cells can include expression control sequences, such as an origin of replication, a promoter (e.g., the CMV promoter, the HSV tk promoter or pgk (phosphoglycerate kinase promoter)), an enhancer (Queen et al. (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins are available, for instance, from the American Type Culture Collection Catalogue of Cell Lines and Hybridomas (7th edition, 1992). One example of mammalian cells for expression of a Bt toxin receptor and assessing Bt toxin cytotoxicity mediated by the receptor, is human embryonic kidney 293 cells. See U.S. Pat. No. 5,693,491, herein incorporated by reference.

Appropriate vectors for expressing polypeptides disclosed herein in insect cells are usually derived from the SF9 baculovirus. Suitable insect cell lines include mosquito larvae, silkworm, armyworm, moth and Drosophila cell lines such as a Schneider cell line (Schneider et al. (1987) J Embryol. Exp. Morphol. 27: 353-365). One embodiment contemplates a cell-free polypeptide expression system.

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague et al. (1983) J. Virol. 45:773-781). Additionally, gene sequences to control replication in the host cell may be incorporated into the vector such as those found in bovine papilloma virus-type vectors. Saveria-Campo, M., Bovine Papilloma Virus DNA a Eukaryotic Cloning Vector in DNA Cloning Vol. II a Practical Approach, D. M. Glover, ed., IRL Pres, Arlington, Va. pp. 213-238 (1985).

In a particular embodiment, it may be desirable to negatively control receptor binding; particularly, when toxicity to a cell is no longer desired or if it is desired to reduce toxicity to a lower level. In this case, ligand-receptor polypeptide binding assays may be used to screen for compounds that bind to the receptor polypeptides but do not confer toxicity to a cell expressing the receptor. The examples of a molecule that can be used to block ligand binding include an antibody that specifically recognizes the ligand binding domain of the receptor polypeptides such that ligand binding is decreased or prevented as desired.

In another embodiment, receptor polynucleotide or polypeptide expression could be altered, for example, reduction by mediating RNA interference (RNAi), including the use of a silencing element directed against specific receptor polynucleotide sequence. Silencing elements can include, but are not limited to, a sense suppression element, an antisense suppression element, a double stranded RNA (dsRNA), a siRNA, a amiRNA, a miRNA, or a hairpin suppression element. Inhibition of expression of coding sequences of a receptor polynucleotide or polypeptide by a silencing element may occur by providing exogenous nucleic acid silencing element constructs, for example, a dsRNA, to an insect. Silencing element constructs contain at least one silencing element targeting the receptor polynucleotide.

In particular embodiments, reducing the polynucleotide level and/or the polypeptide level of the target sequence in a pest results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate target insect. Methods to assay for the level of the RNA transcript include, but are not limited to qRT-PCR, Northern blotting, RT-PCR, and digital PCR.

In specific embodiments, the silencing element has 100% sequence identity to the target receptor polynucleotide. In other embodiments, the silencing element has homology to the target polypeptide have at least 50%, 60%, 70%, 80%, 85%, 90%, 91%, 92%, 93%, 94%, 95%, 96%, 97%, 98%, 99%, or greater sequence identity to a region of the target polynucleotide, where the sequence identity to the target polynucleotide need only be sufficient to decrease expression of the target receptor polynucleotide. Generally, sequences of at least 19 nucleotides, 21 nucleotides, 24 nucleotides, 50 nucleotides, 100 nucleotides, 200 nucleotides, or greater may be used.

Fragments and variants of the disclosed nucleotide sequences and polypeptides encoded thereby are contemplated herein. By “fragment” is intended a portion of the nucleotide sequence, or a portion of the amino acid sequence, and hence a portion of the polypeptide encoded thereby. Fragments of a nucleotide sequence may encode polypeptide fragments that retain the biological activity of the native polypeptide and, for example, bind Bt toxins. Alternatively, fragments of a nucleotide sequence that are useful as hybridization probes. Thus, fragments of a nucleotide sequence may range from at least about 20 nucleotides, about 50 nucleotides, about 100 nucleotides, and up to the full-length nucleotide sequence encoding the polypeptides of the embodiment.

A fragment of a Bt toxin receptor nucleotide sequence that encodes a biologically active portion of a Bt toxin receptor polypeptide may encode at least 15, 25, 30, 50, 100, 150, 200 or 250 contiguous amino acids, or up to the total number of amino acids present in a full-length Bt toxin receptor polypeptide. Fragments of a Bt toxin receptor nucleotide sequence that are useful as hybridization probes for PCR primers generally need not encode a biologically active portion of a Bt toxin receptor polypeptide.

Thus, a fragment of a Bt toxin receptor nucleotide sequence may encode a biologically active portion of a Bt toxin receptor polypeptide, or it may be a fragment that can be used as a hybridization probe or PCR primer using methods disclosed below. A biologically active portion of a Bt toxin receptor polypeptide can be prepared by isolating a portion of one of the Bt toxin receptor nucleotide sequences, expressing the encoded portion of the Bt toxin receptor polypeptide (e.g., by recombinant expression in vitro), and assessing the activity of the encoded portion of the Bt toxin receptor polypeptide. Nucleic acid molecules that are fragments of a Bt toxin receptor nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400, 1500, 2000, or 2500 nucleotides, or up to the number of nucleotides present in a full-length Bt toxin receptor nucleotide sequence disclosed herein. By “variants” is intended substantially similar sequences. For nucleotide sequences, conservative variants include those sequences that, because of the degeneracy of the genetic code, encode the amino acid sequence of one of the Bt toxin receptor polypeptides. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis, but which still encode a Bt toxin receptor protein. Generally, variants of a particular nucleotide sequence of the embodiment will have at least about 40%, 50%, 60%, 65%, 70%, generally at least about 75%, 80%, 85%, 86%, 87%, 88, 89%, such as at least about 90%, 91%, 92%, 93%, 94%, 95%, 96%, 97%, for example at least about 98%, 99% or more sequence identity to that particular nucleotide sequence as determined by sequence alignment programs described elsewhere herein using default parameters.

Variants of a particular nucleotide sequence of the embodiment (i.e., the reference nucleotide sequence) can also be evaluated by comparison of the percent sequence identity between the polypeptide encoded by a variant nucleotide sequence and the polypeptide encoded by the reference nucleotide sequence. Thus, for example, isolated nucleic acids that encode a polypeptide with a given percent sequence identity to the polypeptide of SEQ ID NOs: 2, 4, 6, 8, 10, or 12 are disclosed. Percent sequence identity between any two polypeptides can be calculated using sequence alignment programs described elsewhere herein using default parameters. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, generally at least about 75%, 80%, 85%, such as at least about 90%, 91%, 92%, 93%, 94%, 95%, 96%, 97%, for example at least about 98%, 99% or more sequence identity.

Variants of a particular nucleotide sequence disclosed herein (i.e., the reference nucleotide sequence) can also be evaluated by comparison of the percent sequence identity between the polypeptide encoded by a variant nucleotide sequence and the polypeptide encoded by the reference nucleotide sequence. Thus, for example, isolated nucleic acids that encode a polypeptide with a given percent sequence identity to the polypeptide of SEQ ID NOs: 2, 4, 6, 8, 10, or 12 are disclosed. Percent sequence identity between any two polypeptides can be calculated using sequence alignment programs described elsewhere herein using default parameters. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant polypeptides and polynucleotides in the present embodiment also include homologous and orthologous polypeptide sequences. Variant proteins contemplated herein are biologically active, that is they continue to possess the desired biological activity of the native protein, that is, activity as described herein (for example, Bt toxin binding activity). Such variants may result from, for example, genetic polymorphism or from human manipulation. Biologically active variants of a native Bt toxin receptor protein will have at least about 40%, 50%, 60%, 65%, 70%, generally at least about 75%, 80%, 85%, 86%, 87%, 88%, 89%, such as at least about 90%, 91%, 92%, 93%, 94%, 95%, 96%, 97%, for example at least about 98%, 99% or more sequence identity to the amino acid sequence for the native protein as determined by sequence alignment programs described elsewhere herein using default parameters. A biologically active variant of a protein may differ from that protein by as few as 1-15 amino acid residues, as few as 1-10, such as 6-10, as few as 5, as few as 4, 3, 2, or even 1 amino acid residue.

In one embodiment, the variants of a target receptor can be used for high throughput screening, such as, but not limited to, phage display as reported in Fernandez et al (2008) Peptides, 29(2) 324-329). See also Guo et al. Appl Microbiol Biotechnology. 93(3) 1249-1256. This screening can be used to develop increased toxicity of an insecticide, or to screen for a novel site of action. The high throughput screen can also be applied to screening insects or insect populations for altered susceptibility to an insecticide. Furthermore, more than one variant, fragment, receptor, or the combination of variants, fragments, or receptors can be used in one large, but multiple screening assay.

The polypeptides of the embodiment may be altered in various ways including amino acid substitutions, deletions, truncations, and insertions. Methods for such manipulations are generally known in the art. For example, amino acid sequence variants of the Bt toxin receptor polypeptides can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be made.

Thus, the genes and nucleotide sequences contemplated herein include both the naturally occurring sequences as well as mutant forms. Likewise, the proteins of the embodiment encompass naturally occurring proteins as well as variations and modified forms thereof. Such variants will continue to possess the desired toxin binding activity. The mutations that may be made in the DNA encoding the variant must not place the sequence out of reading frame and in some embodiments, will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. For example, it is recognized that at least about 10, 20, 50, 100, 150, 200, 250, 300, 350, 400, 450, 500, 550, 600, 650, 700, 750, 800, 850, 900, 950, and up to 960 amino acids may be deleted from the N-terminus of a polypeptide that has the amino acid sequence set forth in SEQ ID NOs: 2, 4, 6, 8, 10, or 12, and still retain binding function. It is further recognized that at least about 10, 20, 30, 40, 50, 60, 70, 80, 90, 100, 110, and up to 119 amino acids may be deleted from the C-terminus of a polypeptide that has the amino acid sequence set forth in SEQ ID NOs: 2, 4, 6, 8, 10, or 12, and still retain binding function. Deletion variants encompass polypeptides having these deletions. It is recognized that deletion variants that retain binding function encompass polypeptides having these N-terminal or C-terminal deletions, or having any deletion combination thereof at both the C- and the N-termini. In one embodiment, a deletion, insertion, and/or substitution of the protein sequence may alter or signify an alteration in susceptibility to a Bt toxin.

The exact effect of the substitution, deletion, or insertion in advance of doing so, one skilled in the art will appreciate that the effect will be evaluated by routine screening assays. That is, the activity can be evaluated by receptor binding and/or toxicity assays. See, for example, U.S. Pat. No. 5,693,491; Keeton et al. (1998) Appl. Environ. Microbiol. 64(6):2158-2165; Francis et al. (1997) Insect Biochem. Mol. Biol. 27(6):541-550; Keeton et al. (1997) Appl. Environ. Microbiol. 63(9):3419-3425; Vadlamudi et al. (1995) J. Biol. Chem. 270(10):5490-5494; Ihara et al. (1998) Comparative Biochem. Physiol. B 120:197-204; and Nagamatsu et al. (1998) Biosci. Biotechnol. Biochem. 62(4):727-734; each of which is herein incorporated by reference.

Variant nucleotide sequences and polypeptides also encompass sequences and polypeptides derived from a mutagenic and recombinogenic procedure such as DNA shuffling. With such a procedure, one or more different toxin receptor coding sequences can be manipulated to create a new toxin receptor, including but not limited to a new Bt toxin receptor, possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the Bt toxin receptor genes and other known Bt toxin receptor genes to obtain a new gene coding for a polypeptide with an improved property of interest, such as an increased ligand affinity in the case of a receptor. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,448.

Where the receptor polypeptides are expressed in a cell and associated with the cell membrane (for example, by a transmembrane segment), in order for the receptor to bind a desired ligand, for example a Cry2A toxin, the receptor's ligand binding domain must be available to the ligand. In this aspect, it is recognized that the native Bt toxin receptor is oriented such that the toxin binding site is available extracellularly.

Accordingly, in methods comprising use of intact cells, the embodiment provides cell surface Bt-toxin receptors. By a “cell surface Bt toxin receptor” is intended a membrane-bound receptor polypeptide comprising at least one extracellular Bt toxin binding site. A cell surface receptor of the embodiment comprises an appropriate combination of signal sequences and transmembrane segments for guiding and retaining the receptor at the cell membrane such that that toxin binding site is available extracellularly. Where native Bt toxin receptors are used for expression, deduction of the composition and configuration of the signal sequences and transmembrane segments, it is not necessary to ensure the appropriate topology of the polypeptide for displaying the toxin binding site extracellularly. As an alternative to native signal and transmembrane sequences, heterologous signal and transmembrane sequences could be utilized to produce a cell surface receptor polypeptide.

It is recognized that it may be of interest to generate Bt toxin receptors that are capable of interacting with the receptor's ligands intracellularly in the cytoplasm, in the nucleus or other organelles, in other subcellular spaces; or in the extracellular space. Accordingly, the embodiment encompasses variants of the receptors, wherein one or more of the segments of the receptor polypeptide is modified to target the polypeptide to a desired intra- or extracellular location.

Also encompassed are receptor fragments and variants that are useful, among other things, as binding antagonists that will compete with a cell surface receptor disclosed herein. Such a fragment or variant can, for example, bind a toxin but not be able to confer toxicity to a particular cell. In this aspect, the embodiment provides secreted Bt toxin receptors, i.e. receptors that are not membrane bound. In another embodiment, receptor fragments and variants are useful, among other things, as binding antagonists that have a synergistic relationship to a Bt toxin. The secreted receptors can contain a heterologous or homologous signal sequence facilitating their secretion from the cell expressing the receptors; and further comprise a secretion variation in the region corresponding to transmembrane segments. By “secretion variation” is intended that amino acids corresponding to a transmembrane segment of a membrane bound receptor comprise one or more deletions, substitutions, insertions, or any combination thereof; such that the region no longer retains the requisite hydrophobicity to serve as a transmembrane segment. Sequence alterations to create a secretion variation can be tested by confirming secretion of the polypeptide comprising the variation from the cell expressing the polypeptide.

The polypeptides of the embodiment can be purified from cells that naturally express them, purified from cells that have been altered to express them (e.g., recombinant host cells) or synthesized using polypeptide synthesis techniques. In one embodiment, the polypeptide is produced by recombinant DNA methods. In such methods a nucleic acid molecule encoding the polypeptide is cloned into an expression vector as described more fully herein and expressed in an appropriate host cell according to known methods in the art. The polypeptide is then isolated from cells using polypeptide purification techniques. Alternatively, the polypeptide or fragment can be synthesized using peptide synthesis methods.

Heterologous polypeptides in which one or more polypeptides are fused with at least one polypeptide of interest are also contemplated herein. One embodiment encompasses fusion polypeptides in which a heterologous polypeptide of interest has an amino acid sequence that is not substantially homologous to the receptor polypeptide. In this embodiment, the receptor polypeptide and the polypeptide of interest may or may not be operatively linked. An example of operative linkage is fusion in-frame so that a single polypeptide is produced upon translation. Such fusion polypeptides can, for example, facilitate the purification of a recombinant polypeptide.

In another embodiment, the fused polypeptide of interest may contain a heterologous signal sequence at the N-terminus facilitating its secretion from specific host cells. The expression and secretion of the polypeptide can thereby be increased by use of the heterologous signal sequence.

The embodiment is also directed to polypeptides in which one or more domains in the polypeptide described herein are operatively linked to heterologous domains having homologous functions. Thus, the toxin binding domain can be replaced with a toxin binding domain for other toxins. Thereby, the toxin specificity of the receptor is based on a toxin binding domain other than the domain encoded by Bt toxin receptor but other characteristics of the polypeptide, for example, membrane localization and topology is based on the Bt toxin receptor of SEQ ID NO: 2, 4, 6, 8, 10, or 12.

Alternatively, the native Bt toxin binding domain may be retained while additional heterologous ligand binding domains, including but not limited to heterologous toxin binding domains are comprised by the receptor. Thus, fusion polypeptides in which a polypeptide of interest is a heterologous polypeptide comprising a heterologous toxin binding domains are also contemplated herein. Examples of heterologous polypeptides comprising Cry1 toxin binding domains include, but are not limited to those disclosed in Knight et al (1994) Mol. Micro. 11: 429-436; Lee et al. (1996) Appl. Environ. Micro. 63: 2845-2849; Gill et al. (1995) J. Biol. Chem. 270: 27277-27282; Garczynski et al. (1991) Appl. Environ. Microbiol. 10: 2816-2820; Vadlamudi et al. (1995) J. Biol. Chem. 270(10):5490-4, and U.S. Pat. No. 5,693,491.

The Bt toxin receptor polypeptides of SEQ ID NO: 2, 4, 6, 8, 10, or 12 may also be fused with other members of the ABC transporter superfamily. Such fusion polypeptides could provide an important reflection of the binding properties of the members of the superfamily. Such combinations could be further used to extend the range of applicability of these molecules in a wide range of systems or species that might not otherwise be amenable to native or relatively homologous polypeptides. The fusion constructs could be substituted into systems in which a native construct would not be functional because of species specific constraints. Hybrid constructs may further exhibit desirable or unusual characteristics otherwise unavailable with the combinations of native polypeptides.

Polypeptide variants contemplated herein include those containing mutations that either enhance or decrease one or more domain functions. For example, in the toxin binding domain, a mutation may be introduced that increases or decreases the sensitivity of the domain to a specific toxin.

As an alternative to the introduction of mutations, an increase in activity may be achieved by increasing the copy number of ligand binding domains. Thus, the embodiment also encompasses receptor polypeptides in which the toxin binding domain is provided in more than one copy.

The embodiment further encompasses cells containing receptor expression vectors comprising the Bt toxin receptor sequences, and fragments and variants thereof. The expression vector can contain one or more expression cassettes used to transform a cell of interest. Transcription of these genes can be placed under the control of a constitutive or inducible promoter (for example, tissue- or cell cycle-preferred).

Where more than one expression cassette is utilized, the cassette that is additional to the cassette comprising at least one receptor sequence may comprise a receptor sequence disclosed herein or any other desired sequence.

The nucleotide sequences disclosed herein can be used to isolate homologous sequences in insect species other than Helicoverpa zea, Chrysodeixis includens, Spodoptera frugiperda, or Ostrinia nubilalis, particularly other lepidopteran species, more particularly other Noctuidae or Crambidae species.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS 4:11-17; the local alignment of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. USA 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990), supra. BLAST nucleotide searches can be performed with the BLASTN program, score=100, wordlength=12, to obtain nucleotide sequences homologous to a nucleotide sequence encoding a protein of the embodiment. BLAST protein searches can be performed with the BLASTX program, score=50, wordlength=3, to obtain amino acid sequences homologous to a protein or polypeptide of the embodiment. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.hlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62. See Henikoff and Henikoff (1989) Proc. Natl. Acad. Sci. USA 89:10915.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, at least 80%, at least 90%, or at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, at least 70%, at least 80%, at least 90%, such as at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid sequence is immunologically cross reactive with the polypeptide encoded by the second nucleic acid sequence.

(e)(ii) The term “substantial identity” in the context of a peptide indicates that a peptide comprises a sequence with at least 70% sequence identity to a reference sequence, at least 80%, at least 85%, such as at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The nucleotide sequences disclosed herein may be used to isolate corresponding sequences from other organisms, particularly other insects, more particularly other lepidopteran species. In this manner, methods such as PCR, hybridization, and the like can be used to identify such sequences based on their sequence homology to the sequences set forth herein. Additionally, a transcriptome can be used to identify such sequences based on their sequence homology to the sequences set forth herein. See Yinu et al (2012). Plos One, 7(8): e43713. Sequences isolated based on their sequence identity to the entire Bt toxin receptor sequences set forth herein or to fragments thereof are contemplated herein. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences which encode polypeptides having Bt toxin receptor activity and which hybridize under stringent conditions to the H. zea Bt toxin receptor sequences disclosed herein, or to fragments thereof, are contemplated herein.

In a PCR-based approach, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

Degenerate bases, otherwise known as wobbles, are equimolar mixtures of two or more different bases at a given position within a sequence. Since the genetic code is degenerate (e.g., histidine could be encoded by CAC or CAT), an oligo probe may be prepared with wobbles at the degenerate sites (e.g., for histidine CAY is used where Y=C+T). There are eleven standard wobbles mixtures. The standard code letters for specifying a wobble are as follows: R=A+G; Y=C+T; M=A+C; K=G+T; S=C+G; W=A+T; B=C+G+T; D=A+G+T; H=A+C+T; V=A+C+G; and N=A+C+G+T.

Degenerate bases are used to produce degenerate probes and primers. Degenerate bases are often incorporated into oligonucleotide probes or primers designed to hybridize to an unknown gene that encodes a known amino acid sequence. They may also be used in probes or primers that are designed based upon regions of homology between similar genes in order to identify a previously unknown ortholog. Oligonucleotides with wobbles are also useful in random mutagenesis and combinatorial chemistry.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the Bt toxin receptor sequences. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire Bt toxin receptor sequences disclosed herein, or one or more portions thereof, may be used as a probe capable of specifically hybridizing to corresponding Bt toxin receptor sequences and messenger RNAs. To achieve specific hybridization under a variety of conditions, such probes include sequences that are unique among Bt toxin receptor sequences and are at least about 10 nucleotides in length, or at least about 20 nucleotides in length. Such probes may be used to amplify corresponding Bt toxin receptor sequences from a chosen plant organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989)Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, such as less than 500 nucleotides in length.

Typically, stringent conditions will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (%GC)−0.61 (% form)—500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode for a Bt toxin receptor protein and which hybridize under stringent conditions to the Bt toxin receptor sequences disclosed herein, or to fragments thereof, are encompassed herein.

The compositions and screening methods of the embodiment are useful for identifying cells expressing the Bt toxin receptors, variants and homologues thereof. Such identification could utilize detection methods at the protein level, such as ligand-receptor binding, or at the nucleotide level. Detection of the polypeptide could be in situ by means of in situ hybridization of tissue sections but may also be analyzed by bulk polypeptide purification and subsequent analysis by Western blot or immunological assay of a bulk preparation. Alternatively, receptor gene expression can be detected at the nucleic acid level by techniques known to those of ordinary skill in any art using complimentary polynucleotides to assess the levels of genomic DNA, mRNA, and the like. As an example, PCR primers complimentary to the nucleic acid of interest can be used to identify the level of expression. Tissues and cells identified as expressing the receptor sequences of the embodiment are determined to be susceptible to toxins that bind the receptor polypeptides.

Where the source of the cells identified to express the receptor polypeptides is an organism, for example an insect plant pest, the organism is determined to be susceptible to toxins capable of binding the polypeptides. In a particular embodiment, identification is in a lepidopteran plant pest expressing a Bt toxin receptor set forth herein.

The embodiment encompasses antibody preparations with specificity against the receptor polypeptides. In further embodiments, the antibodies are used to detect receptor expression in a cell.

In one aspect, the embodiment is drawn to compositions and methods for modulating susceptibility of plant pests to Bt toxins. However, it is recognized that the methods and compositions may be used for modulating susceptibility of any cell or organism to the toxins. By “modulating” is intended that the susceptibility of a cell or organism to the cytotoxic effects of the toxin is increased or decreased. By “susceptibility” is intended that the viability of a cell contacted with the toxin is decreased. Thus the embodiment encompasses expressing the cell surface receptor polypeptides to increase susceptibility of a target cell or organ to Bt toxins. Such increases in toxin susceptibility are useful for medical and veterinary purposes in which eradication or reduction of viability of a group of cells is desired. Such increases in susceptibility are also useful for agricultural applications in which eradication or reduction of populations of particular plant pests is desired.

Plant pests of interest include, but are not limited to insects, nematodes, and the like. Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

In one embodiment, the methods comprise creating a genetically edited or modified insect, or colony thereof. The polynucleotide sequence of the target receptor may be used to knockout or mutate the target receptor polynucleotide in an insect by means known to those skilled in the art, including, but not limited to use of a Cas9/CRISPR system, TALENs, homologous recombination, and viral transformation. See Ma et al (2014), Scientific Reports, 4: 4489; Daimon et al (2013), Development, Growth, and Differentiation, 56(1): 14-25; and Eggleston et al (2001) BMC Genetics, 2:11. A knockout or mutation of the target receptor polynucleotide should presumably result in an insect having reduced or altered susceptibility to a Bt toxin or other pesticide. The resulting resistant insect, or colony thereof, can be used to screen potential new active toxins or other agents for new or different sites of action. Current toxins can also be characterized using a resistant insect line.

In one embodiment, one or more polynucleotides as set forth in SEQ ID NOs: 1, 3, 5, 7, 9, 11, or 13-15, or an expression construct comprising a sequence as set forth in SEQ ID NOs: 1, 3, 5, 7, 9, 11, or 13-15, and compositions comprising said sequences, may be edited or inserted by genome editing using double stranded break inducing agent, such as a CRISPR/Cas9 system. In one embodiment, the genomic DNA sequence set forth in SEQ ID NOs: 13-15 may be edited or inserted by genome editing using double stranded break inducing agent, such as a CRISPR/Cas9 system.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times-also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas endonuclease relates to a Cas protein encoded by a Cas gene, wherein said Cas protein is capable of introducing a double strand break into a DNA target sequence. The Cas endonuclease is guided by a guide polynucleotide to recognize and optionally introduce a double strand break at a specific target site into the genome of a cell (See U.S. Patent Application Publication No. 2015/0082478). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

In one embodiment, the methods comprise creating an insect, or colony thereof, wherein the target gene is edited so that it is no longer functional. The polynucleotide sequence of the target gene can be used to knockout the target gene polynucleotide in an insect by means known to those skilled in the art, including, but not limited to use of a Cas9/CRISPR system, TALENs, homologous recombination, and viral transformation. See Ma et al (2014), Scientific Reports, 4: 4489; Daimon et al (2013), Development, Growth, and Differentiation, 56(1): 14-25; and Eggleston et al (2001) BMC Genetics, 2:11.

In one embodiment, the methods relate to methods that result in rescue of resistance achieved through the target receptor polynucleotide expression (e.g., targeting a negative regulatory element by RNAi) or a reverse mutation.

In one embodiment, the methods relate to creating an insect colony resistant to Cry2 Bt toxins. A colony can be made through genetically modification methods or the target receptor polynucleotide can be used to screen for mutants, insects lacking the target receptor polynucleotide, or any other genetic variants. Subsequent screening and selection on a Cry2 toxin should result in a Cry2 resistant colony that may be used as described herein. The methods include, but are not limited to, feeding the insects leaf material from maize plants expressing insecticides or purified insecticides applied to an artificial diet, and selecting individuals that survived exposure. The methods may further involve transferring the surviving insects to a standard diet that lacks insecticide to allow the survivors to complete development. The methods can further involve allowing the surviving insects to mate to maintain the colony with selection periodically applied in subsequent generations by feeding the insects leaf material from maize plants expressing insecticides or purified insecticides and selecting surviving insects, and therefore fixing resistance by eliminating individuals that do not carry homozygous resistance alleles.

One embodiment encompasses a method of screening insect populations for altered levels of susceptibility to an insecticide, including a resistance monitoring assay. An assay for screening altered levels of susceptibility includes, but is not limited to, assaying a target receptor gene DNA sequence, RNA transcript, polypeptide, or activity of the target receptor polypeptide. Methods for assaying include, but are not limited to DNA sequencing, Southern blotting, northern blotting, RNA sequencing, PCR, RT-PCR, qPCR, qRT-PCR, protein sequencing, western blotting, mass spectrometry identification, antibody preparation and detection, and enzymatic assays. A change in sequence in a DNA, RNA transcript, or polypeptide can indicate a resistant insect. Also, a change in the amount or abundance of an RNA, a polypeptide, or an enzymatic activity of a target receptor polypeptide can indicate a resistant insect. In one embodiment, the method includes screening an insect under selection to increase efficiency of selection for a receptor-mediated resistance. In another embodiment, the method comprises screening for a mutation or altered sequence in a disclosed polypeptide receptor of SEQ ID NOs: 2, 4, 6, 8, 10, or 12, a change in expression of SEQ ID NOs: 2, 4, 6, 8, 10, or 12, or a change in expression of SEQ ID NOs: 1, 3, 5, 7, 9, 11, or a complement thereof, wherein the change indicates receptor-mediated resistance to a toxin. In another embodiment, the method relates to screening an insect for an ABC transporter gene or gene product, transcript, or polypeptide sequence that is different from a native non-resistant insect sequence. In one embodiment, an insect with an altered or mutated sequence is further exposed to an insecticidal toxin, wherein the insecticidal toxin has the same site of action as a Cry 2 toxin. The use of screening for a receptor allows for efficient receptor-mediated resistance selection to create a resistant insect colony.

In one embodiment, the method relates to a method for monitoring insect resistance or altered levels of susceptibility to a Cry toxin in a field comprising assaying for altered levels of susceptibility or insect resistance, which may include, but not limited to, assaying a target receptor gene DNA sequence, RNA transcript, polypeptide, or activity of the target receptor polypeptide. Methods for assaying include, but are not limited to DNA sequencing, Southern blotting, northern blotting, RNA sequencing, PCR, RT-PCR, qPCR, qRT-PCR, protein sequencing, western blotting, mass spectrometry identification, antibody preparation and detection, and enzymatic assays. A change in sequence in the DNA, RNA transcript, or polypeptide can indicate a resistant insect. Also, a change in the amount or abundance of an RNA, a polypeptide, or an enzymatic activity of a target receptor polypeptide can indicate a resistant insect. In another embodiment, the method comprises screening for a mutation or altered sequence in a disclosed polypeptide receptor of SEQ ID NOs: 2, 4, 6, 8, 10, or 12, a change in expression of SEQ ID NOs: 2, 4, 6, 8, 10, or 12, or a change in expression of SEQ ID NOs: 1, 3, 5, 7, 9, 11, or a complement thereof, wherein the change indicates receptor-mediated resistance to a toxin. In a further embodiment, the method relates to applying an insecticidal agent to an area surrounding the environment of an insect or an insect population having an ABC transporter gene or gene product sequence that is different from a native sequence, wherein the insecticidal agent has a different mode of action compared to a Cry2 Bt toxin. In further embodiment, the method comprises implementing an insect management resistance (IRM) plan. In one embodiment, an IRM plan may include, but not limited to, adding refuge or additional refuge, rotation of crops, planting additional natural refuge, and applying a insecticide with a different site of action.

In one embodiment, the methods comprise an assay kit to monitor resistance. The simple kits can be used in the field or in a lab to screen for the presence of resistant insects. In preferred embodiments, an antibody raised against SEQ ID NOs: 2, 4, 6, 8, 10, or 12 may be used to determine levels of, or the presence of, absence of or change in concentration of SEQ ID NOs: 2, 4, 6, 8, 10, or 12 in an insect population. In another embodiment, an assessment of SEQ ID NOs: 1, 3, 5, 7, 9, 11, or 13-15 is performed, either to assess sequence changes in an insect or insect population target receptor sequence or for expression changes relative to a control or for sequence variation. Molecular techniques are common to those skilled in the art to accomplish the resistance monitoring in a kit, such as but not limited to PCR, RT-PCR, qRT-PCR, Southern blotting, Northern blotting, and others.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing embodiment has been described in some detail by way of illustration and example for purposes of clarity of understanding, certain changes and modifications may be practiced within the scope of the appended claims.

The following examples are offered by way of illustration and not by way of limitation.

### EXPERIMENTAL

### Example 1

### Specific Binding of Bt Toxin to Lepidopteran Insects

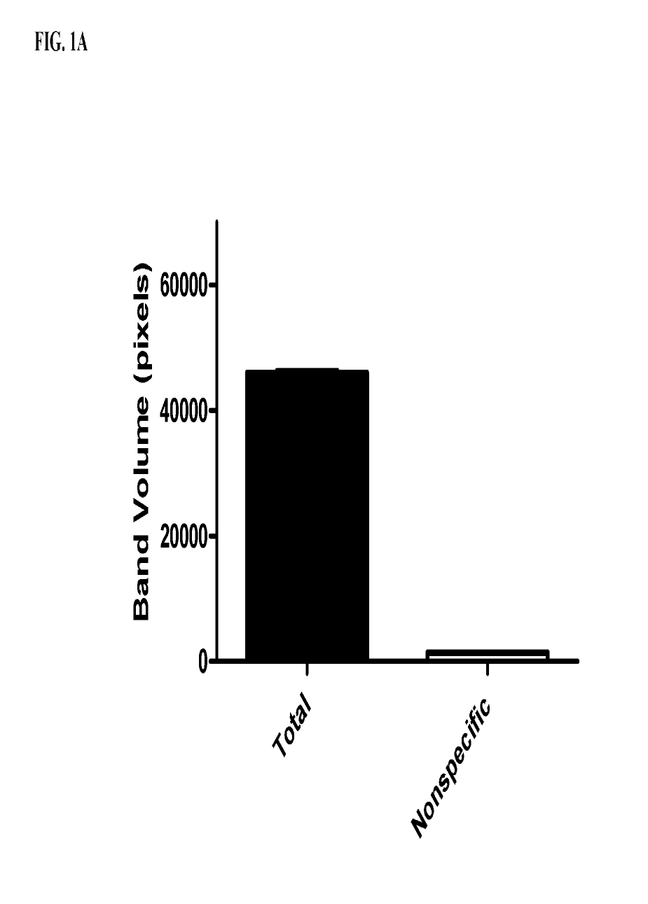

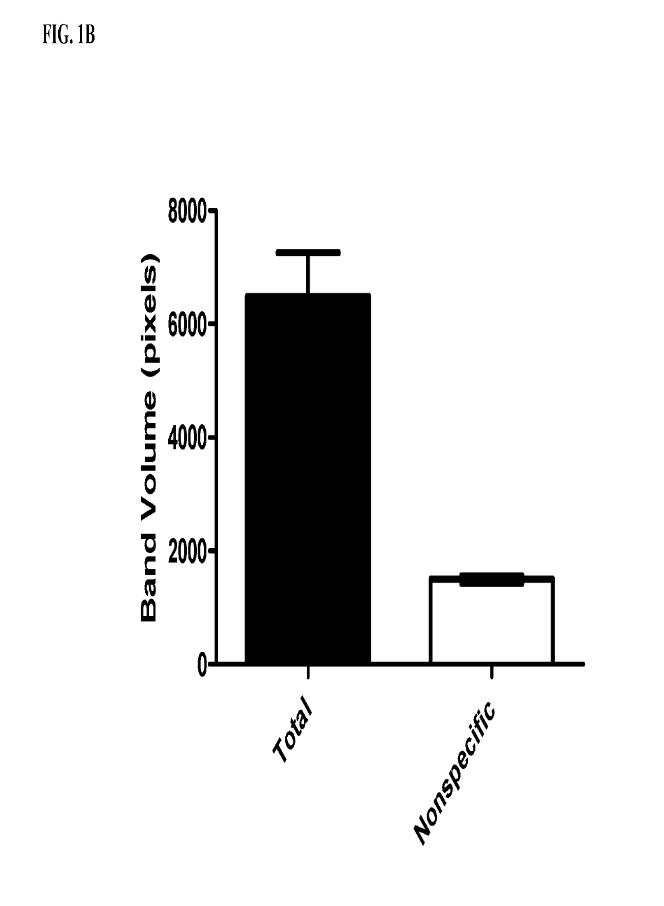

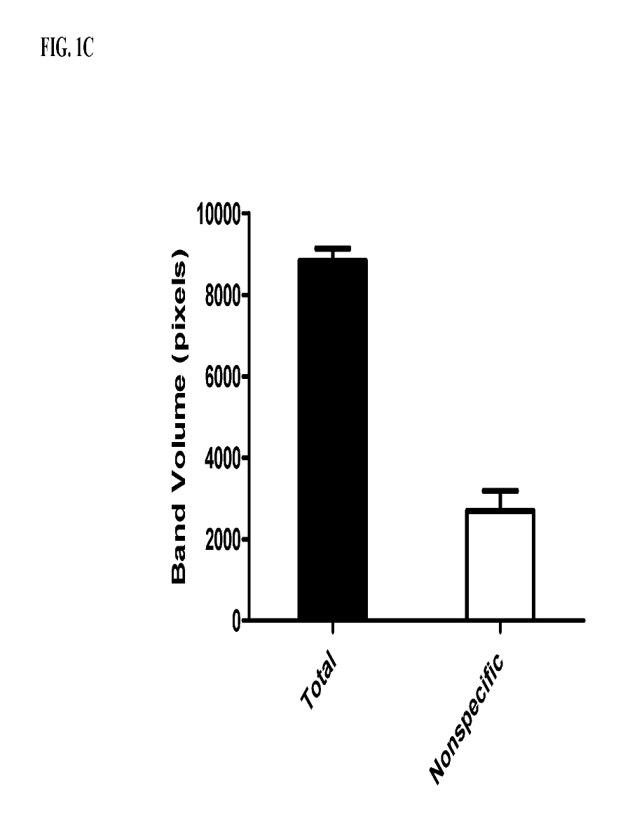

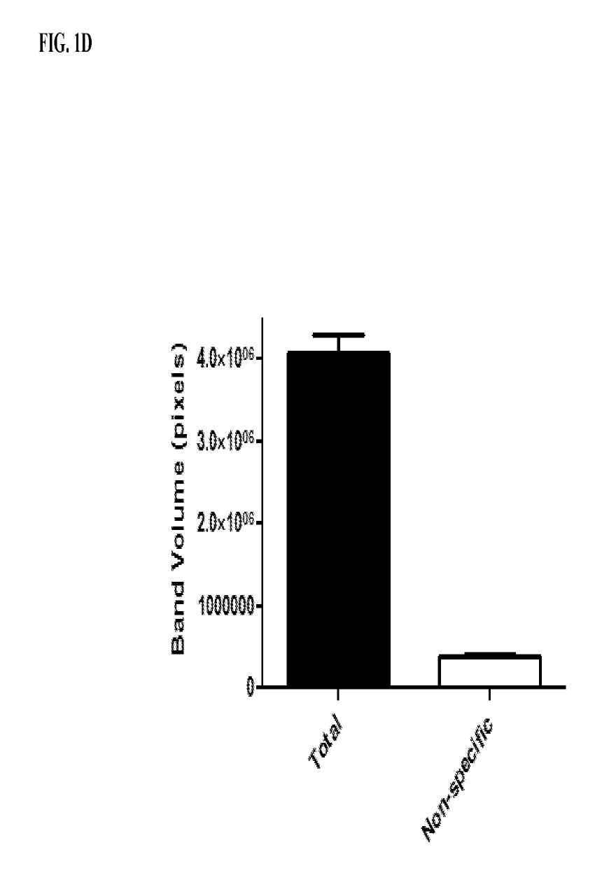

Midguts from fourth instar Helicoverpa zea, Ostrinia nubilalis, Spodoptera frugiperda, and Chrysodeixis includens larvae were isolated for brush border membrane vesicle (BBMV) preparation using the protocol by Wolfersberger et al. (1987) Comp. Biochem. Physiol. 86A:301-308. An in-solution competitive binding assay was performed using 40 μg (protein content) of BBMVs from H. zea (corn earworm) and O. nubilalis and 10 nM IP2.127 (SEQ ID NO: 21) labeled with Alexa-488 fluorescence molecule to measure specific binding of IP2.127 to H. zea or O. nubilalis. An in-solution competitive binding assay was performed using 20 μg (protein content) of BBMVs from S. frugiperda (fall armyworm) and 10 nM IP2.127 labeled with Alexa-488 fluorescence molecule to measure specific binding of IP 2.127 to S. frugiperda. Binding buffer used for IP2.127 binding was a sodium carbonate buffer consisting of 50 mM sodium carbonate/HCl pH 9.6, 150 mM NaCl, 0.1% Tween 20. An in-solution binding competitive binding assay was performed using 40 μg (protein content) of BBMVs from C. includens (soybean looper) and 5 nM IP2.127 labeled with Alexa-488 fluorescence molecule to measure specific binding of IP2.127 to C. includens. Binding buffer used for IP2.127 binding in C. includens was a CAPS buffer consisting of 20 mM CAPS pH 10.5, 150 mM NaCl, and 0.1% Tween 20. FIG. 1A shows the homologous competition of IP2.127 in H. zea, FIG. 1B shows the homologous competition of IP2.127 in O. nubilalis, FIG. 1C shows the homologous competition of IP2.127 in S. frugiperda, and FIG. 1D shows the homologous competition of IP2.127 in C. includens.

### Example 2

### Isolation of Lepidopteran Bt Toxin Receptor

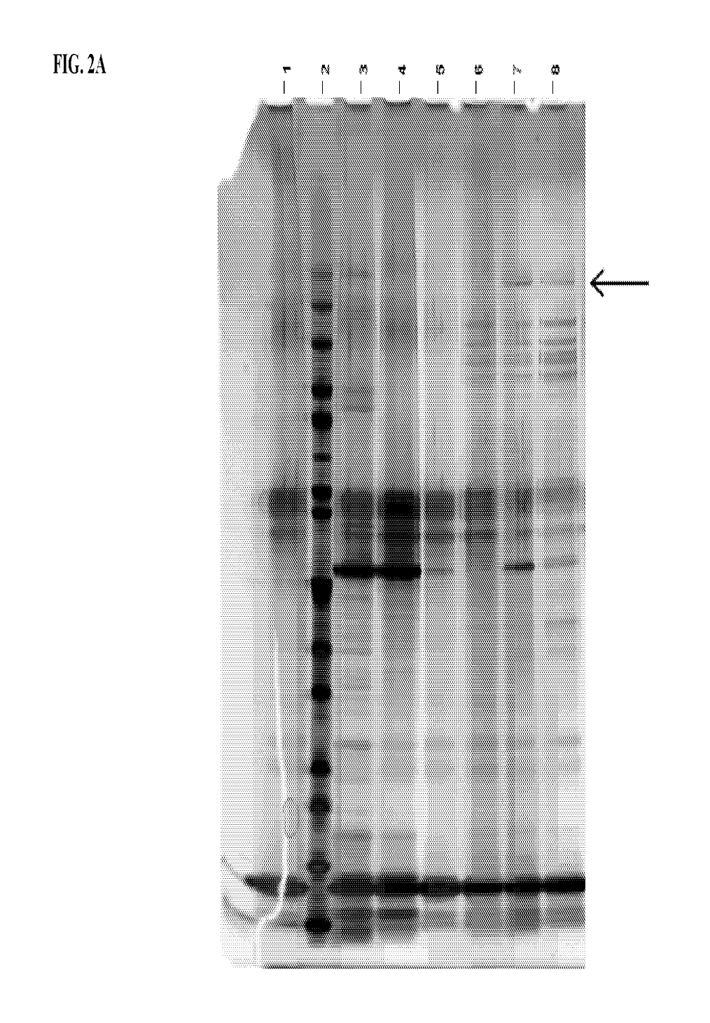

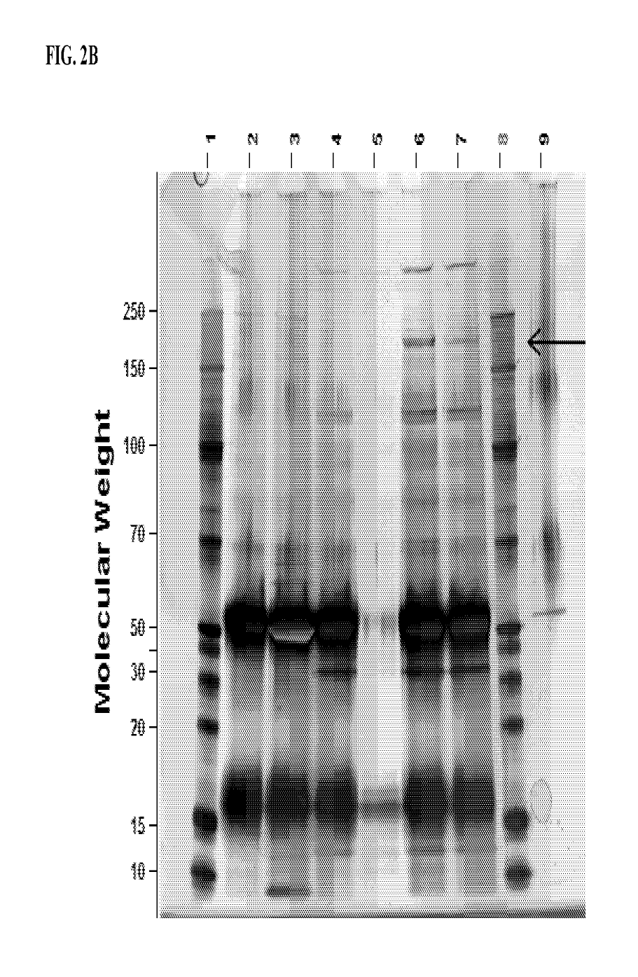

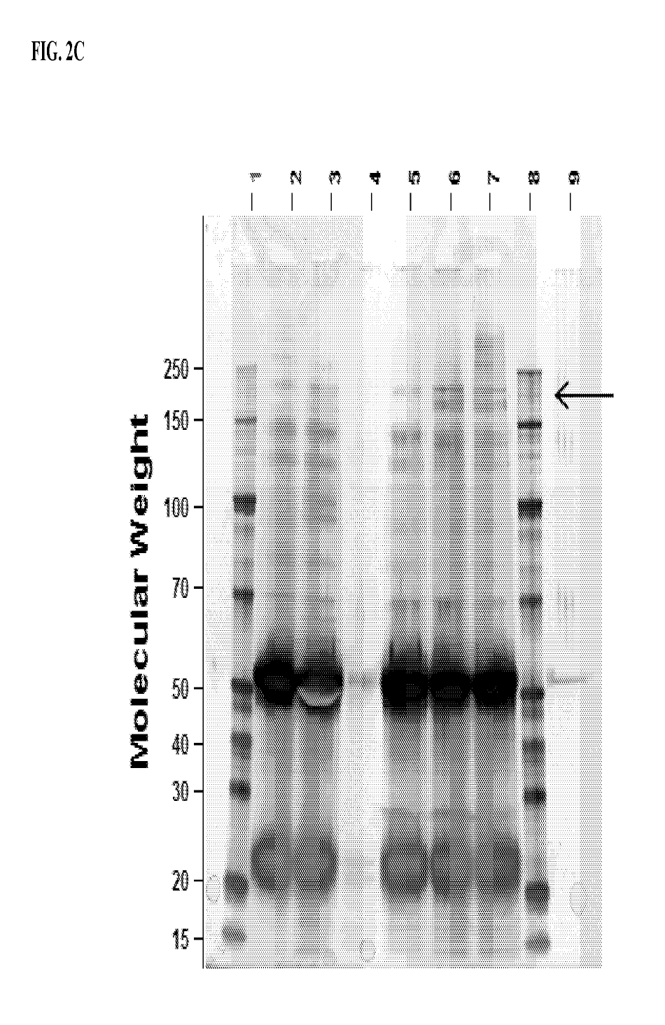

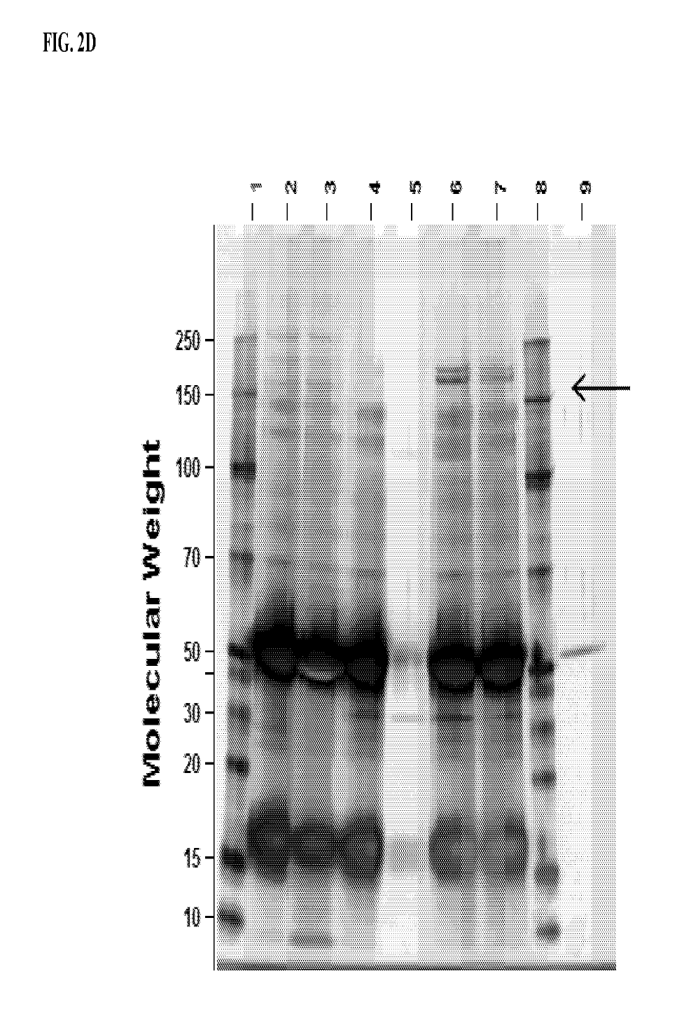

A solution binding assay was done using H. zea BBMVs with biotin labeled IP2.127 (SEQ ID NO: 21). The binding assay was followed by the detergent (Triton X100®) extraction of proteins from BBMVs bound to the biotin-labeled IP2.127. The proteins bound to biotin labeled IP2.127 were then “co-precipitated” (co-isolated) using Dynabeads® MyOne™ Streptavidin T1 (Life Technologies # 65601) which binds the biotin-labeled IP2.127 and proteins bound to biotin labeled IP2.127 while unbound proteins are washed away. The samples are then separated by SDS-PAGE and stained to visualize protein bands. FIG. 2A shows the gel of the isolated proteins with an arrow indicating to the unique protein that was selected for mass spectrometry in H. zea.

Solution binding assays were done using one of each of O. nubilalis, S. frugiperda, or C. includens BBMVs with IP2.127. The binding assays were followed by the detergent (Triton X100®) extraction of proteins from BBMVs bound to the IP2.127. The proteins bound to IP2.127 were then “co-immunoprecipitated” (co-isolated) using Dynabeads® Protein G (Life Technologies # 10007D), which were bound to IP2.127 antibody. The beads bound to antibody then bind the IP2.127 and proteins bound to IP2.127 and unbound proteins are washed away. The samples are then separated by SDS-PAGE and stained to visualize protein bands. FIG. 2B shows the gel of the co-isolated proteins from O. nubilalis with an arrow pointing to the unique protein sent for mass spectrometry. FIG. 2C shows the gel of the co-isolated proteins from S. frugiperda with an arrow pointing to the unique protein sent for mass spectrometry. FIG. 2D shows the gel of the co-isolated proteins from C. includens with an arrow pointing to the unique protein sent for mass spectrometry.

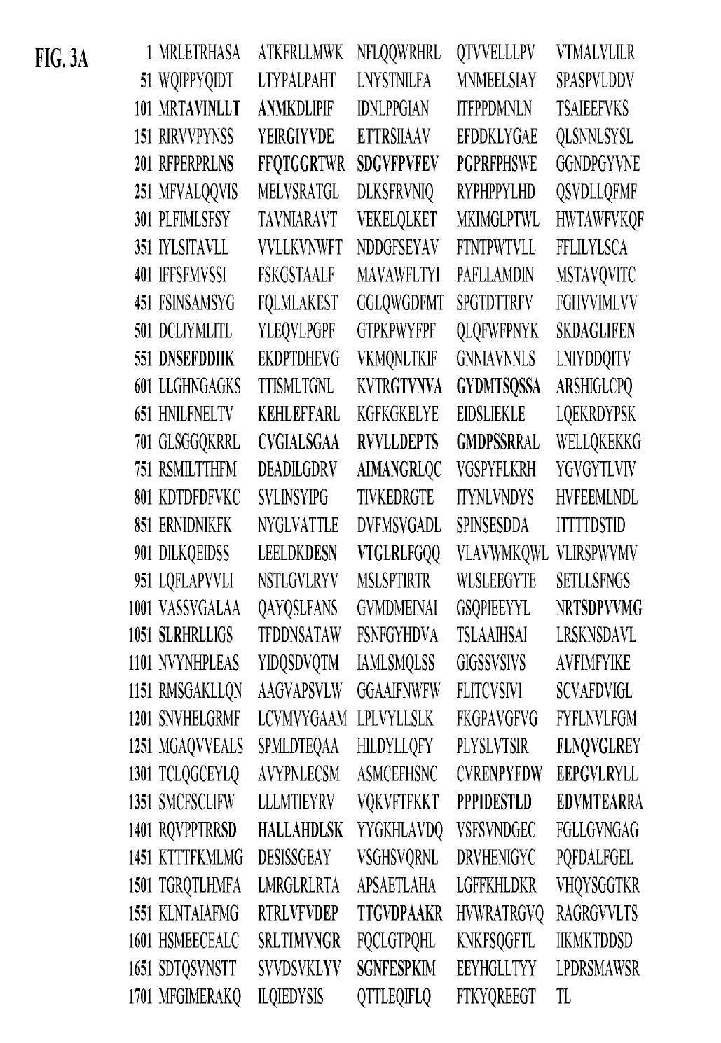

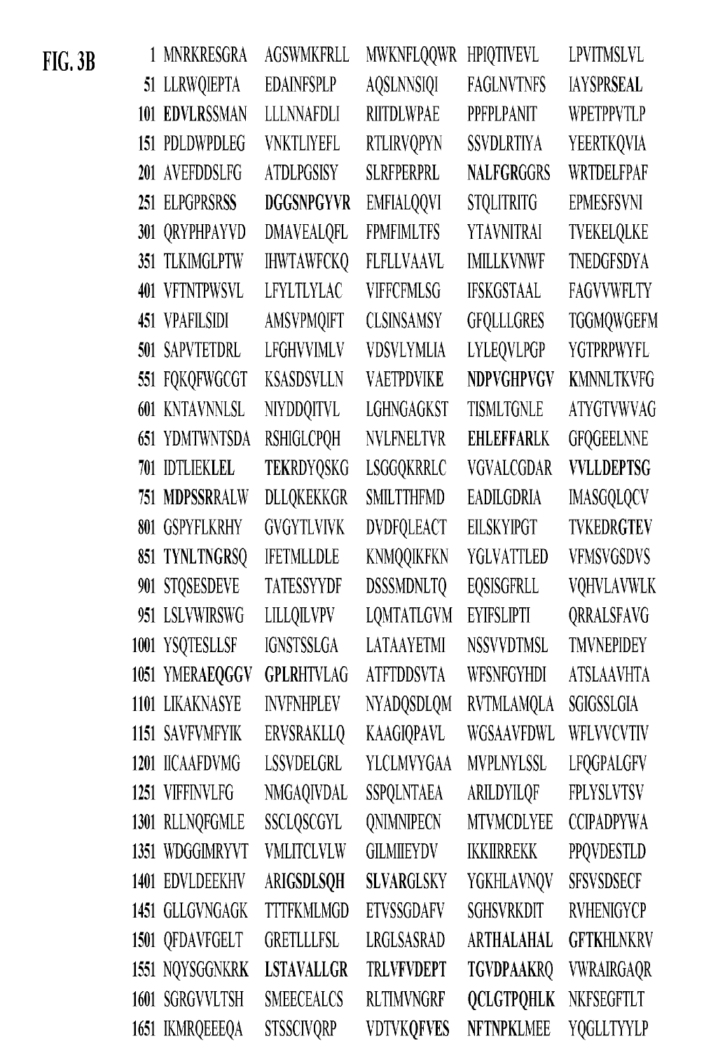

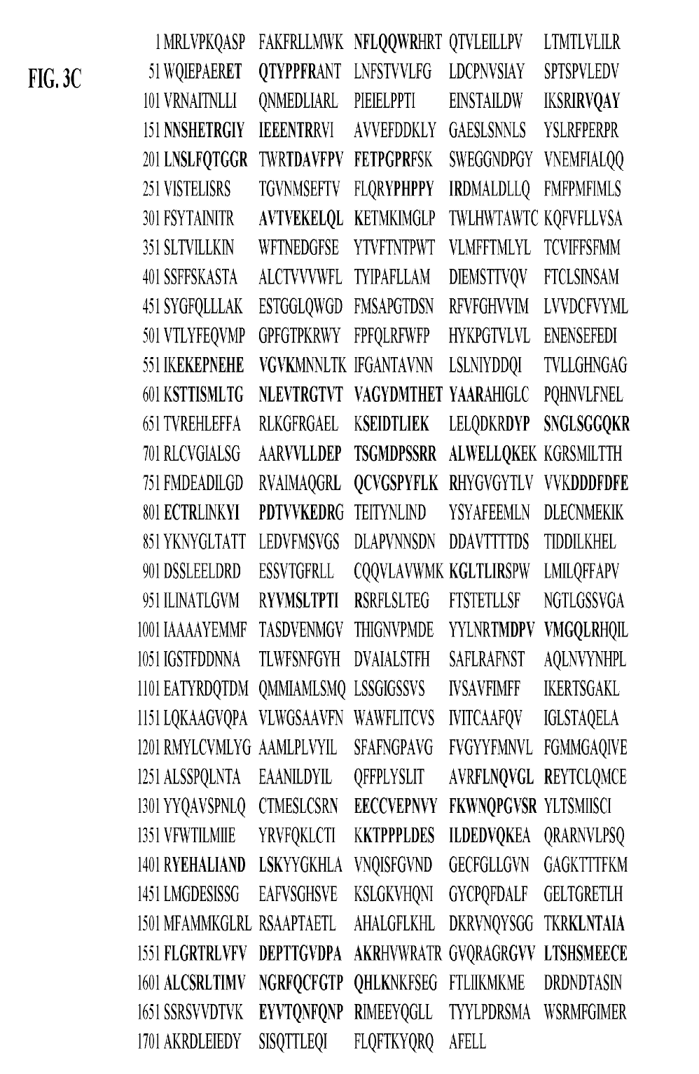

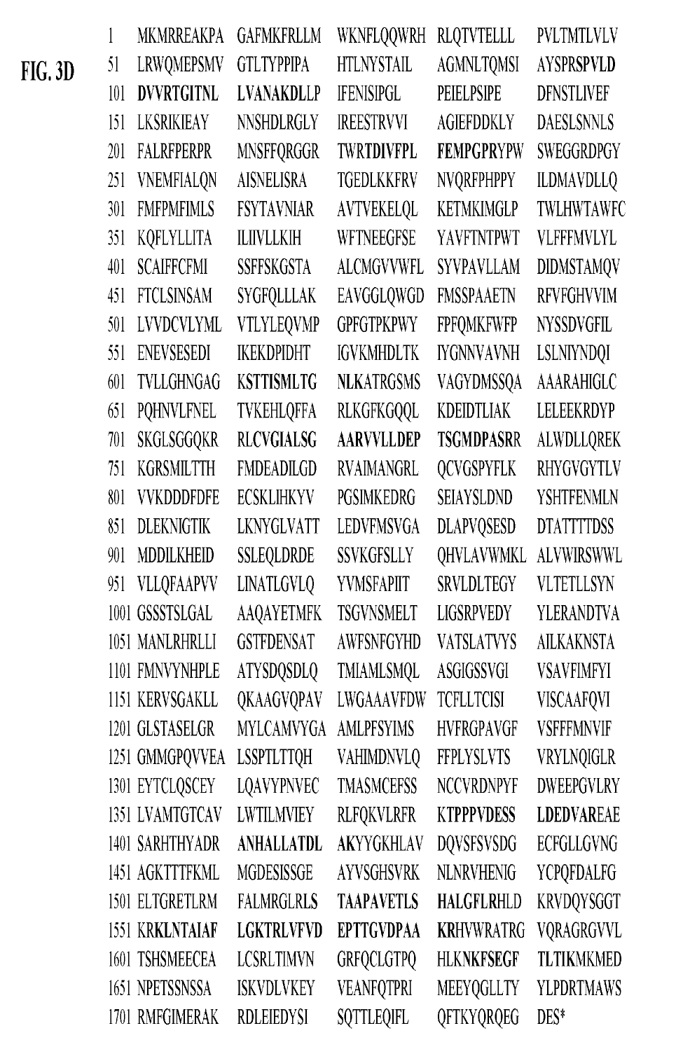

The unique band was excised from the SDS-PAGE gel, digested by trypsin, and the resulting peptides analyzed by mass spectrometry for identification. The resulting peptide sequences from the protein band were identified for H. zea as SEQ ID NO: 2 with 13% peptide sequence coverage, for O. nubilalis as SEQ ID NO: 4 with 9% peptide sequence coverage, for S. frugiperda as SEQ ID NO: 8 with 21% peptide sequence coverage, and for C. includens as SEQ ID NO: 10 with 9% peptide sequence coverage (see FIGS. 3a, 3b, 3c, and 3d respectively). Open reading frames (ORFs) were identified in Vector NTI® Suite software (available from Informax, Inc., Bethesda, Md.) to determine a nucleotide sequence encoding SEQ ID NO: 2 for H. zea, and SEQ ID NO: 4 for O. nubilalis SEQ ID NO: 8 for S. frugiperda, and SEQ ID NO: 10 for C. inlcudens. The cDNA sequences encoding the identified region were blasted to a proprietary H. zea, O. nubilalis, S. frugiperda and C. includens transcriptome. Table 1 indicates cDNA sequences identified and homologous sequences from other corn pests. Further sequence analysis was conducted to verify the cDNA sequence and to isolate variants by isolating cDNA from Helicoverpa zea, Ostrinia nubilalis, and Chrysodeixis includens and cloning the receptor sequences using species specific primers (SEQ ID NOs: 22-27) matching to the transcriptome sequences into E. coli (for methods see Maniatis, T., E. F. Fritsch, and J. Sambrook. Molecular Cloning, a Laboratory Manual, 1982). The cloned cDNA sequences were sequenced, and the nucleotide sequences are set forth in SEQ ID NOs: 16-19.

TABLE 1
The receptor nucleotide coding sequence for H. zea,
SEQ ID NO: 2, was identified by mass spectrometry.
This sequence was then blasted against proprietary
sequence databases and the remaining sequences
were identified with >50% homology.

| | | | % |
|---|---|---|---|
| Gene ID | Species | Seq no. | homology |
| ATP-binding cassette sub- | Helicoverpa | Seq no. 001 | 100 |
| family A member 3 XnoC3 | zea | | |
| ATP-binding cassette sub- | Ostrinia | Seq no. 003 | 66.1 |
| family A member 3 5NOC3 | nubilalis | | |
| ATP-binding cassette sub- | Spodoptera | Seq no. 005 | 74.5 |
| family A member 3 XnoC3 | frugiperda | | |
| Atp-binding cassette sub-family | Ostrinia | Seq no. 007 | 66.1 |
| G member/ARP2_G246 XnoC3 | nubilalis | | |

That which is claimed:

1. A heterologous polynucleotide comprising:
a) the nucleotide sequence set forth in SEQ ID NO: 1, 3, 5, 7, or 9;
b) a nucleotide sequence encoding the amino acid sequence set forth in SEQ ID NO: 2, 4, 6, 8, or 10;
c) a nucleotide sequence having at least about 90% sequence identity to the nucleotide sequence set forth in SEQ ID NO: 1, 3, 5, 7, or 9, wherein said nucleotide sequence having at least about 90% sequence identity to the nucleotide sequence set forth in SEQ ID NO: 1, 3, 5, 7, or 9 encodes a polypeptide having Bt toxin binding activity;
d) a nucleotide sequence that hybridizes to the complement of the nucleotide sequence set forth in SEQ ID NO: 1, 3, 5, 7, or 9 under stringent conditions, wherein said nucleotide sequence that hybridizes to the complement of the nucleotide sequence set forth in SEQ ID NO: 1, 3, 5, 7, or 9 under stringent conditions encodes a polypeptide having Bt toxin binding activity; or
e) a nucleotide sequence complementary to at least one nucleotide sequence set forth in a), b), c), and d).

2. The heterologous polynucleotide of claim 1, wherein said heterologous polynucleotide comprises a nucleotide sequence encoding a polypeptide having Cry toxin binding activity.

3. The heterologous polynucleotide of claim 2, wherein said heterologous polynucleotide comprises a nucleotide sequence encoding a polypeptide having Cry2A binding activity.

4. A heterologous polypeptide having the amino acid sequence comprising:
a) the amino acid sequence set forth in SEQ ID NO: 2, 4, 6, 8, or 10; or b) a variant of the amino acid sequence set forth in SEQ ID NO: 2, 4, 6, 8, or 10, wherein said sequence variant has Bt toxin binding activity and shares at least about 90% sequence identity with the amino acid sequence set forth in SEQ ID NO: 2, 4, 6, 8, or 10;

5. A polypeptide complex comprising a heterologous polypeptide comprising the heterologous polypeptide of claim 4, wherein said heterologous polypeptide is bound to a Cry toxin.

6. The heterologous polypeptide of claim 5, wherein said heterologous polypeptide is bound to a Cry2A toxin.

7. An antibody preparation specific for the polypeptide of claim 4.

8. An expression cassette comprising the nucleotide sequence of claim 1 operably linked to a promoter.

9. A host cell comprising the expression cassette of claim 8.

10. The host cell of claim 9, wherein said cell is a microorganism.

11. The host cell of claim 10 wherein said microorganism is selected from the group consisting of yeast and bacteria.

12. The host cell of claim 9, wherein said cell is an insect cell.

13. The host cell of claim 9, wherein said cell is a mammalian cell.

14. A transformed cell of interest having stably incorporated within its genome a heterologous nucleotide sequence comprising:
a) the nucleotide sequence set forth in SEQ ID NO: 1, 3, 5, 7, or 9;
b) a nucleotide sequence encoding the amino acid sequence set forth in SEQ ID NO: 2, 4, 6, 8, or 10;
c) a nucleotide sequence having at least about 90% sequence identity to the nucleotide sequence set forth in SEQ ID NO: 1, 3, 5, 7, or 9, wherein said nucleotide sequence having at least about 90% sequence identity to the nucleotide sequence set forth in SEQ ID NO: 1, 3, 5, 7, or 9 encodes a polypeptide having Bt toxin binding activity;
d) a nucleotide sequence that hybridizes to the complement of the nucleotide sequence set forth in SEQ ID NO: 1, 3, 5, 7, or 9 under stringent conditions, wherein said nucleotide sequence that hybridizes to the complement of the nucleotide sequence set forth in SEQ ID NO: 1, 3, 5, 7, or 9 under stringent conditions encodes a polypeptide having Bt toxin binding activity; or
e) a nucleotide sequence complementary to at least one nucleotide sequences et forth in a), b), c), or d).

15. The transformed cell of claim 14, wherein said cell is a plant cell.

16. The transformed cell of claim 15, wherein said plant cell is monocotyledonous.

17. A method for identifying compounds that bind to the polypeptide of claim 4, said method comprising:
a) contacting the polypeptide of claim 4 with one or more test compounds; and
b) determining whether the test compound binds to the polypeptide.

18. A method for screening test compounds to identify a compound that binds to the polypeptide of claim 4, said method comprising:
a) contacting the host cell of claim 9 with one or more test compounds; and
b) determining whether the test compound binds to the polypeptide of claim 4.

19. A method for generating a variant of SEQ ID NO: 1, 3, 5, 7, or 9 comprising modifying the endogenous sequence of SEQ ID NO: 1, 3, 5, 7, or 9 in an insect by a transgenic technique.

20. The method of claim 19, wherein the insect expresses a mutated variant polypeptide of SEQ ID NO: 2, 4, 6, 8, or 10.

21. A method for selecting altered susceptibility of an insect, said method comprising:
a) identifying in an insect:
i) alterations of the nucleotide sequence of SEQ ID NO: 1, 3, 5, 7, or 9;
ii) alterations of the amino acid sequence of SEQ ID NO: 2, 4, 6, 8, or 10; or
iii) changes in expression of SEQ ID NOs: 1, 2, 3, 4, 5, 6, 7, 8, 9, or 10; and
b) selecting for altered susceptibility of said insect.

22. The method of claim 21, wherein the insect is a transgenic insect.

23. A method for altering the susceptibility of an insect to an insecticide, comprising feeding an insect a silencing element, wherein the silencing element comprises at least 19 consecutive nucleotides of:
a) the nucleotide sequence set forth in SEQ ID NO: 1, 3, 5, 7, or 9;
b) a nucleotide sequence encoding the amino acid sequence set forth in SEQ ID NO: 2, 4, 6, 8, or 10;
c) a nucleotide sequence having at least about 90% sequence identity to the nucleotide sequence set forth in SEQ ID NO: 1, 3, 5, 7, or 9, wherein said nucleotide sequence having at least about 90% sequence identity to the nucleotide sequence set forth in SEQ ID NO: 1, 3, 5, 7, or 9 encodes a polypeptide having Bt toxin binding activity;
d) a nucleotide sequence that hybridizes to the complement of the nucleotide sequence set forth in SEQ ID NO: 1, 3, 5, 7, or 9 under stringent conditions, wherein said nucleotide sequence that hybridizes to the complement of the nucleotide sequence set forth in SEQ ID NO: 1, 3, 5, 7, or 9 under stringent conditions encodes a polypeptide having Bt toxin binding activity; or
e) a nucleotide sequence complementary to at least one nucleotide sequence set forth in a), b), c), and d),
wherein feeding the insect a silencing element alters the susceptibility of the insect to an insecticide.

24. A kit for screening populations of insects, wherein said kit comprises a means for assaying an altered level of expression of a sequence of an ABC transporter gene or gene product, wherein the alteration indicates an insect resistant to an insecticide.

25. A method for isolating a receptor of an insect midgut toxin comprising:
a) dissecting an insect to collect midgut tissue;
b) performing a membrane enrichment step on the midgut tissue, such as a BBMV preparation;
b) performing an in-solution binding assay on the enriched membrane; and
c) performing an affinity purification, wherein the toxin is the affinity purification target.


SEQUENCE LISTING (27 sequences)

SEQ ID NO: 1 - DNA, 5229 bp, Helicoverpa zea
atgagattagaaacgaggcacgctagtgccgccaccaagttccgcctgctcatgtggaagaactttctgcagcaatggagacatcggctacaaactgtcgtagagctactattgccagtcgtgacaatggcgctggtgctcatccttcggtggcagataccgccttatcaaatcgatacactcacttaccccgcgttgccagcgcacacactcaactactctaccaatatcctttttgccatgaatatggaagaattatcaattgcatactccccggcaagtccagtgttagatgatgtaatgagaactgctgttattaatttactaacagccaatatgaaagatctgattcctatttttattgataacttaccaccggggatagccaatataacatttccaccagatatgaacttaaatacgtcggccattgaggagttcgtgaagtcacgaatacgagtcgtaccttataacagcagttatgaaataagagggatctacgttgacgaagaaactacacgcagcattatcgctgccgtcgagtttgacgataaactatatggagcagaacagttgtcaaataatttatcttattcgctacgttttcctgagagaccccgtctcaattcgtttttccaaactggagggcgcacctggagatctgacggagtattcccggttttcgaagtgcctgggcctagatttcctcactcgtgggaaggtggtaatgacccaggttacgttaacgaaatgttcgtggcacttcaacaagttatttccatggagctggtatcaagggcaaccgggttggacttgaagtcatttagggtgaacatacagaggtacccgcacccgccgtaccttcacgaccagtcagtggatctgctgcaatttatgttccccctgttcatcatgttgagcttcagctacactgctgtcaacattgcacgggcggtcacagttgagaaggaattgcaattaaaggaaactatgaaaattatgggcctccccacatggttgcactggacagcatggtttgttaaacagtttatctacctatcaatcacagctgttctgctagttgtgttgctaaaggtaaattggtttactaacgacgatggcttcagcgaatatgctgtatttactaatacaccttggacggttttgctattcttcttgatactgtatttatcttgcgcgatatttttttctttcatggtaagcagtatattttcaaaaggtagtacggccgcgttgtttatggcggtggcatggttcctcacttacatccctgctttcctcctggccatggatatcaatatgtcgactgcggtgcaggtcatcacatgcttcagtattaactctgcgatgtcctatggtttccaactaatgctcgctaaggaaagcactggagggctgcagtggggcgacttcatgacgtcaccagggacggacaccacgcgcttcgtgttcggccacgtggtcatcatgctggtagtggactgcctcatctacatgctgatcaccctctaccttgaacaagtgctaccaggccccttcgggactcccaaaccttggtacttccccttccagttgcagttctggttcccaaattataaatcgaaagatgctggattaattttcgaaaatgataatagtgaattcgatgatattataaaagaaaaggatcccacagaccacgaagttggtgttaaaatgcaaaatttaacaaaaatctttgggaataacatagctgttaacaatttatctttgaatatctatgacgaccaaatcacagttttacttggtcataatggtgctggcaaatcaactacaatatctatgttaacaggaaatttaaaggtaactcgcgggacagtgaacgttgccggatatgatatgacttctcaaagctccgcagcccgttcccacattggattgtgtcctcagcacaatatactgtttaacgaactcacggtcaaagaacatttggaattctttgctagactaaaaggatttaaaggcaaagaactgtatgaagagatagactcacttattgaaaaattggaactacaggagaagcgtgactacccctcaaaaggtctgtcaggaggtcagaagcgtcgtctttgtgttggtattgctctgagtggggcggcacgagtggtcttactcgacgaacctacgtctggcatggatccttcatctcgtcgagcactgtgggaactcttacagaaggaaaagaaaggtcgctcgatgatcctgacgactcattttatggacgaagcagatattctcggcgacagagtggctataatggcaaacggtagactgcaatgcgtgggctctccgtatttcctcaaacgtcattatggcgtcgggtataccctggtgatcgttaaggacacagacttcgactttgtgaaatgctccgtacttatcaatagctatattcctggtactattgttaaagaagatcgaggaacggaaatcacttataatttggtaaacgattactcacacgtttttgaagaaatgttgaatgatttggaaagaaatattgataacatcaaatttaaaaactatggtttagttgctactacattagaagatgtctttatgtccgttggtgcagacttaagtccaattaattccgaatctgacgatgctattactactactactgactcgactatcgatgatatattaaaacaagaaatcgattcatctttggaagaactggataaggacgagagtaacgtgacgggcctccgcttgttcggtcagcaagtgctggctgtatggatgaagcagtggctggtgctgatccgctcgccatgggtcatggtactgcagtttttggcgccagtggtactcatcaactccacgctaggagttctgcgttacgtcatgtctttatcaccgaccattagaactaggtggttgtcgttggaagaagggtatacggaaagcgaaactctgctcagcttcaacggtagtgtagcgtcatcagtgggtgccctagccgcgcaagcataccaaagcctgttcgccaattctggtgttatggacatggaaatcaacgctatcggaagccagccaatagaagaatattatctaaatagaacaagtgatcccgttgtgatgggttcgctgcggcaccgcttgctgataggctccacatttgacgacaactctgctaccgcctggttcagtaactttggctaccacgatgttgctacatcgcttgcggcaatccactcagctattctcagatctaaaaactctgatgcagtactcaatgtatataatcatccgttggaagcttcgtatatagatcagagtgacgtgcagactatgatagctatgttgtccatgcagttgtcctctggcatcggcagtagtgtgagcattgttagtgcggttttcatcatgttttatatcaaggaacgtatgtcgggggcaaaacttctacaaaatgcagcaggcgtggcgccttctgtgctgtggggcggcgcagcgatcttcaattggttttggttcctcatcacttgtgtttccatcgtcatctcgtgcgtcgcttttgatgtcatcgggttatcgaacgtgcatgaattaggtcgaatgtttttgtgcgtcatggtatacggtgcggcgatgttgccattagtgtaccttttgtcgcttaagttcaagggaccagctgtcggcttcgtgggcttctatttcctcaacgtgcttttcggtatgatgggtgcgcaggtggtggaggcactatcctctcctatgctggacacagagcaagccgcccacatccttgactacttactgcagttctacccgctttacagtcttgtcacttctatcaggtttttaaatcaagtcggcctacgggagtatacttgcttacaaggctgtgaatacttgcaggcagtatacccgaatctagagtgtagcatggcaagcatgtgcgaattccacagtaactgctgcgttcgtgaaaacccatacttcgattgggaggaaccaggcgtcctgaggtacttgctcagtatgtgcttctcctgcctaatcttctggttgctgcttatgaccattgaatacagagtggtgcaaaaggtgttcacattcaagaagactcctcctccaatagacgagagcacgttagacgaggacgtgatgacagaggcgaggcgcgcgcgccaggtgccgccgacacgccgcagcgaccacgcgcttctcgctcacgacctctccaagtactacgggaaacatctcgccgtagaccaagtctcgtttagtgtgaacgacggcgaatgcttcggtctattgggtgtgaatggtgccggaaaaacgaccaccttcaagatgctgatgggtgatgagtccatttcaagcggcgaggcgtatgtctccgggcactcggtgcagaggaatctcgatagagtacacgagaatattggatattgtccgcaatttgacgcattatttggtgagctgacgggtcgccagacactacacatgtttgcgttgatgcgcggcttgcgtttacgcactgcagcaccttcggctgaaacactcgcacatgcgcttggcttcttcaaacatcttgataaaagggtgcatcagtattcaggcggcacgaaacgcaagcttaacacggcgatagcattcatgggacgaacacggcttgtgtttgttgatgagcctaccactggagttgatcccgccgctaaacgccacgtatggcgcgctacccgcggcgtgcagcgagcaggtcgcggcgtggtgctgacgtcacatagtatggaggagtgcgaggcgttgtgttcgcgactgaccatcatggtcaacggtcgcttccagtgtctgggaacgccacaacatctcaagaacaaattttctcaaggctttactttaatcattaaaatgaaaactgacgacagtgacagcgacacgcagtcagtaaacagcactaccagcgtagtagatagtgtcaaactatacgtctctgggaactttgaaagtccaaagataatggaagagtatcatggtcttctaacttactacttgcctgaccgtagcatggcatggtcacgaatgtttggtatcatggagcgcgccaaacagatcttacaaattgaggactacagcatatcgcagactaccctcgaacaaatattcttgcagttcaccaaataccaaagagaagaaggaacgacgttataa

SEQ ID NO: 2 - protein, 1742 aa, Helicoverpa zea
MRLETRHASAATKFRLLMWKNFLQQWRHRLQTVVELLLPVVTMALVLILRWQIPPYQIDTLTYPALPAHTLNYSTNILFAMNMEELSIAYSPASPVLDDVMRTAVINLLTANMKDLIPIFIDNLPPGIANITFPPDMNLNTSAIEEFVKSRIRVVPYNSSYEIRGIYVDEETTRSIIAAVEFDDKLYGAEQLSNNLSYSLRFPERPRLNSFFQTGGRTWRSDGVFPVFEVPGPRFPHSWEGGNDPGYVNEMFVALQQVISMELVSRATGLDLKSFRVNIQRYPHPPYLHDQSVDLLQFMFPLFIMLSFSYTAVNIARAVTVEKELQLKETMKIMGLPTWLHWTAWFVKQFIYLSITAVLLVVLLKVNWFTNDDGFSEYAVFTNTPWTVLLFFLILYLSCAIFFSFMVSSIFSKGSTAALFMAVAWFLTYIPAFLLAMDINMSTAVQVITCFSINSAMSYGFQLMLAKESTGGLQWGDFMTSPGTDTTRFVFGHVVIMLVVDCLIYMLITLYLEQVLPGPFGTPKPWYFPFQLQFWFPNYKSKDAGLIFENDNSEFDDIIKEKDPTDHEVGVKMQNLTKIFGNNIAVNNLSLNIYDDQITVLLGHNGAGKSTTISMLTGNLKVTRGTVNVAGYDMTSQSSAARSHIGLCPQHNILFNELTVKEHLEFFARLKGFKGKELYEEIDSLIEKLELQEKRDYPSKGLSGGQKRRLCVGIALSGAARVVLLDEPTSGMDPSSRRALWELLQKEKKGRSMILTTHFMDEADILGDRVAIMANGRLQCVGSPYFLKRHYGVGYTLVIVKDTDFDFVKCSVLINSYIPGTIVKEDRGTEITYNLVNDYSHVFEEMLNDLERNIDNIKFKNYGLVATTLEDVFMSVGADLSPINSESDDAITTTTDSTIDDILKQEIDSSLEELDKDESNVTGLRLFGQQVLAVWMKQWLVLIRSPWVMVLQFLAPVVLINSTLGVLRYVMSLSPTIRTRWLSLEEGYTESETLLSFNGSVASSVGALAAQAYQSLFANSGVMDMEINAIGSQPIEEYYLNRTSDPVVMGSLRHRLLIGSTFDDNSATAWFSNFGYHDVATSLAAIHSAILRSKNSDAVLNVYNHPLEASYIDQSDVQTMIAMLSMQLSSGIGSSVSIVSAVFIMFYIKERMSGAKLLQNAAGVAPSVLWGGAAIFNWFWFLITCVSIVISCVAFDVIGLSNVHELGRMFLCVMVYGAAMLPLVYLLSLKFKGPAVGFVGFYFLNVLFGMMGAQVVEALSSPMLDTEQAAHILDYLLQFYPLYSLVTSIRFLNQVGLREYTCLQGCEYLQAVYPNLECSMASMCEFHSNCCVRENPYFDWEEPGVLRYLLSMCFSCLIFWLLLMTIEYRVVQKVFTFKKTPPPIDESTLDEDVMTEARRARQVPPTRRSDHALLAHDLSKYYGKHLAVDQVSFSVNDGECFGLLGVNGAGKTTTFKMLMGDESISSGEAYVSGHSVQRNLDRVHENIGYCPQFDALFGELTGRQTLHMFALMRGLRLRTAAPSAETLAHALGFFKHLDKRVHQYSGGTKRKLNTAIAFMGRTRLVFVDEPTTGVDPAAKRHVWRATRGVQRAGRGVVLTSHSMEECEALCSRLTIMVNGRFQCLGTPQHLKNKFSQGFTLIIKMKTDDSDSDTQSVNSTTSVVDSVKLYVSGNFESPKIMEEYHGLLTYYLPDRSMAWSRMFGIMERAKQILQIEDYSISQTTLEQIFLQFTKYQREEGTTL

SEQ ID NO: 3 - DNA, 5265 bp, Ostrinia nubilalis
gtgagtagggaaaatatgaatcggaaaagagaaagcggaagggcggccggatcgtggatgaaattccgtttgctaatgtggaagaattttctgcaacaatggaggcatcctattcaaactatcgtagaggtgttgttgccagttatcacgatgtctctagtgctactgcttcggtggcagattgaacctaccgcagaagatgcgataaacttttcgccattacctgcacaatcactgaataattctatacaaatatttgctggtctcaacgtaactaatttctccattgcatactctccaagaagtgaagccttggaggatgtcttacgtagttccatggcaaacttactgctaaacaatgcgttcgatctaatcagaattatcactgatctgtggcctgctgaaccgccattcccactgccagcgaatataacgtggcctgaaaccccgccagtcacactgcctccagatttagactggcctgatttagagggtgtaaacaaaactctgatttacgagtttctcagaaccttaatacgagttcaaccatacaattctagtgttgatctcagaactatttatgcttacgaagagagaactaagcaagtaatagcagctgtggaatttgatgactctctttttggtgctacggatttgcctggaagtatatcgtattccttaaggtttccggagaggcctcgccttaatgcattatttggtcgaggcggccgatcttggagaacagacgaattgtttcctgcatttgagttacctggaccaagatcccgttcttccgatggaggctccaatcctggctacgtcagggaaatgtttattgcgcttcagcaagtaatatcaacgcaactaataacaagaattactggggagcctatggagtcattctcagtcaatattcagagatatccacaccctgcatatgtcgatgatatggcagtagaggcactccagtttctatttcccatgttcattatgctgacctttagctacacagccgtgaatatcaccagagccatcacagttgagaaggaacttcaacttaaggaaacattgaaaattatgggcttacccacatggatacattggactgcatggttttgcaaacagttcctctttttattggtagcagctgttcttattatgattcttctaaaggttaattggtttacgaacgaagatgggttcagcgattacgccgtatttacaaatacaccatggagtgtcttgctattctatttaacactctacttggcatgcgtgatattcttttgtttcatgctcagtggtatattttcgaaaggtagtacagcagcgctatttgccggtgtggtgtggttcctcacctacgtcccagcgtttatcctgtccattgacattgctatgtcagtccctatgcaaatatttacatgtctcagtatcaactctgcaatgtcttacggattccagctgcttctgggtagagagagcacaggcggtatgcaatggggcgagttcatgtcggcgcccgtgacggagaccgaccggctgctgttcgggcacgtggtaatcatgctggtggtggactcggtgctgtacatgctgatcgcgctgtacctggagcaggtgctgcccggcccctacggcacgcccaggccctggtacttcttattccagaaacaattttgggggtgcggcactaaatctgcatcagattcagtattactaaatgttgctgaaactcctgatgtaataaaggagaacgatccggtgggtcacccagttggagttaaaatgaacaatctgacaaaagtattcggcaagaatactgcagtaaataatttgagtttaaatatatacgacgaccaaattacagttttgctcggccataatggagcaggaaaatctacaactatatcgatgttgacaggtaatttggaggcaacatatgggacagtgtgggtggccggctatgacatgacttggaacaccagtgatgctcgctcacatataggactatgccctcaacataacgtcctgttcaatgagctcaccgtcagggaacatttagaattctttgccagactgaaaggattccaaggcgaagaattgaacaatgaaatagacactcttatcgagaaactagagttgacagagaagcgtgactaccagtccaaaggcttgtccggcggacagaagcggaggctgtgcgttggcgtggcactgtgtggcgacgcgcgagtggtgctccttgacgagcctacgtcgggcatggacccctcctcgcgccgcgctctctgggacctattgcagaaggaaaaaaaaggtcgttccatgatattgacaacgcacttcatggacgaagctgacatcctgggggaccggatcgctattatggcgagcgggcagttgcaatgcgtcggctcgccatacttcctcaagcgacactacggcgtcgggtacactctggtcattgtcaaggatgttgacttccagttggaagcctgcacggagattttaagcaaatatattccaggaactactgttaaggaagataggggaacggaagttacatacaatttgactaacggtcgatctcaaatattcgaaacaatgttattggacctagaaaaaaatatgcaacaaataaaattcaaaaactatggattagttgccacgaccttagaagatgtgtttatgtcagttggatctgatgtatcatccacacagtcagagtcagatgaggtggaaactgcaactgagtcatcgtattacgactttgattcatcttcaatggataacttaacacaagaacagtcgatcagtggttttcgtttgttggtgcaacatgttttggccgtctggctgaagctgtctctggtctggatccggtcctggggcctgattctgctgcagattttggtaccggtgctacaaatgaccgcaacacttggagttatggaatacattttcagtttgataccaactatacagcgcagggcgctatcgtttgccgtaggttattcgcaaacggagagccttttgagtttcatcggcaattccacatcatcactgggggccctagcgactgcagcatacgagacgatgatcaactcgagcgttgtcgacaccatgtcgttgaccatggtcaatgaaccgattgatgaatattatatggaaagggcggaacaaggaggagtgggaccgctgcgccacacggttttggcgggtgccactttcacagatgattcggtaaccgcatggttcagcaacttcggctaccacgacattgccacttcgttagccgccgttcacaccgcgcttatcaaagctaaaaacgcttcgtatgaaatcaacgtgttcaaccatccacttgaagtcaactatgctgatcaaagtgaccttcaaatgcgggtgacgatgctagccatgcaactggcttccggcatcgggagcagtttgggtattgccagtgcagttttcgtcatgttttatattaaggagcgcgtgtctcgagcgaaactgctgcagaaggcggccggcatccagcctgcagttctatggggcagcgcggctgtgttcgactggctgtggttccttgtggtctgcgtcactatcgtcatcatttgtgccgcttttgacgtcatgggcctctcatccgtcgacgaactcggtcgcctatacttgtgcctgatggtgtacggcgctgcgatggtgcctctgaactacctgtcttccctcctgttccaaggcccggcacttggcttcgttgtcatattcttcataaatgtactcttcggaaatatgggggctcagatcgtggacgcactgtcatcgccgcagctaaataccgcagaggcggcgaggatcctggactatatactgcagttctttccactctacagtctcgtaacgtctgtcagactattgaatcagttcggcatgttagagagctcgtgcctacaaagctgtggatacttacagaacattatgaacataccagagtgcaatatgactgttatgtgcgatctctatgaagaatgctgtattcctgcagacccatactgggcctgggacggtggcattatgcgatatgtaactgtcatgctgatcacctgtcttgtattgtggggaatactcatgattatagagtacgacgttattaagaagataatcagacgagagaagaaaccaccacaagttgacgaaagcacactagacgaggatgtattggacgaagagaagcacgtcgcgcgcatcggctccgacctctctcagcacagcctcgtggcgcgcgggctctccaagtactacggcaagcacctggccgtcaatcaagtgtccttcagcgtaagcgactctgaatgcttcggtctgctgggggtgaacggagccggcaagaccaccacgttcaagatgctaatgggcgacgagaccgtctccagcggagacgcgttcgtcagtggccactctgtcaggaaggacatcacacgtgttcatgaaaatatcggatactgcccgcagttcgacgccgtattcggcgaactgacgggccgtgagacgttacttttgttctcgctgctccgcgggctcagcgccagccgcgctgacgctcgcacgcacgcgctggcgcatgcgctcggcttcaccaagcacttgaacaaacgggtaaaccagtattcagggggcaacaaacgcaaattgagcacagcggtagccttactgggccgcactcggttggtgttcgtcgacgaacccaccactggcgttgaccctgcggctaaacgacaggtatggcgtgctatccgaggcgcccagcgttcaggcagaggcgtggtgctgacgtcacatagcatggaggagtgcgaggcgctatgctcgcgcctcaccatcatggtcaacggccgcttccagtgccttggcacccctcaacacctgaaaaacaagttttctgaaggttttactttaactattaaaatgagacaagaagaggagcaagcatcaactagttcatgcattgttcagagacctgtagatacggtcaaacaattcgtcgaatccaacttcactaacccaaaacttatggaagagtatcaaggtttgctgacatactacttgcccgaccgcagcatcgcatggtcaagaatgtttggaatcatggagcaagccaagagagacctccaggtggaggactacagcatttcccaaaccactttagaacagatcttcctacaatttaccaaatatcaacaagaggctgcaattcaataa

SEQ ID NO: 4 - protein, 1754 aa, Ostrinia nubilalis
VSRENMNRKRESGRAAGSWMKFRLLMWKNFLQQWRHPIQTIVEVLLPVITMSLVLLLRWQIEPTAEDAINFSPLPAQSLNNSIQIFAGLNVTNFSIAYSPRSEALEDVLRSSMANLLLNNAFDLIRIITDLWPAEPPFPLPANITWPETPPVTLPPDLDWPDLEGVNKTLIYEFLRTLIRVQPYNSSVDLRTIYAYEERTKQVIAAVEFDDSLFGATDLPGSISYSLRFPERPRLNALFGRGGRSWRTDELFPAFELPGPRSRSSDGGSNPGYVREMFIALQQVISTQLITRITGEPMESFSVNIQRYPHPAYVDDMAVEALQFLFPMFIMLTFSYTAVNITRAITVEKELQLKETLKIMGLPTWIHWTAWFCKQFLFLLVAAVLIMILLKVNWFTNEDGFSDYAVFTNTPWSVLLFYLTLYLACVIFFCFMLSGIFSKGSTAALFAGVVWFLTYVPAFILSIDIAMSVPMQIFTCLSINSAMSYGFQLLLGRESTGGMQWGEFMSAPVTETDRLLFGHVVIMLVVDSVLYMLIALYLEQVLPGPYGTPRPWYFLFQKQFWGCGTKSASDSVLLNVAETPDVIKENDPVGHPVGVKMNNLTKVFGKNTAVNNLSLNIYDDQITVLLGHNGAGKSTTISMLTGNLEATYGTVWVAGYDMTWNTSDARSHIGLCPQHNVLFNELTVREHLEFFARLKGFQGEELNNEIDTLIEKLELTEKRDYQSKGLSGGQKRRLCVGVALCGDARVVLLDEPTSGMDPSSRRALWDLLQKEKKGRSMILTTHFMDEADILGDRIAIMASGQLQCVGSPYFLKRHYGVGYTLVIVKDVDFQLEACTEILSKYIPGTTVKEDRGTEVTYNLTNGRSQIFETMLLDLEKNMQQIKFKNYGLVATTLEDVFMSVGSDVSSTQSESDEVETATESSYYDFDSSSMDNLTQEQSISGFRLLVQHVLAVWLKLSLVWIRSWGLILLQILVPVLQMTATLGVMEYIFSLIPTIQRRALSFAVGYSQTESLLSFIGNSTSSLGALATAAYETMINSSVVDTMSLTMVNEPIDEYYMERAEQGGVGPLRHTVLAGATFTDDSVTAWFSNFGYHDIATSLAAVHTALIKAKNASYEINVFNHPLEVNYADQSDLQMRVTMLAMQLASGIGSSLGIASAVFVMFYIKERVSRAKLLQKAAGIQPAVLWGSAAVFDWLWFLVVCVTIVIICAAFDVMGLSSVDELGRLYLCLMVYGAAMVPLNYLSSLLFQGPALGFVVIFFINVLFGNMGAQIVDALSSPQLNTAEAARILDYILQFFPLYSLVTSVRLLNQFGMLESSCLQSCGYLQNIMNIPECNMTVMCDLYEECCIPADPYWAWDGGIMRYVTVMLITCLVLWGILMIIEYDVIKKIIRREKKPPQVDESTLDEDVLDEEKHVARIGSDLSQHSLVARGLSKYYGKHLAVNQVSFSVSDSECFGLLGVNGAGKTTTFKMLMGDETVSSGDAFVSGHSVRKDITRVHENIGYCPQFDAVFGELTGRETLLLFSLLRGLSASRADARTHALAHALGFTKHLNKRVNQYSGGNKRKLSTAVALLGRTRLVFVDEPTTGVDPAAKRQVWRAIRGAQRSGRGVVLTSHSMEECEALCSRLTIMVNGRFQCLGTPQHLKNKFSEGFTLTIKMRQEEEQASTSSCIVQRPVDTVKQFVESNFTNPKLMEEYQGLLTYYLPDRSIAWSRMFGIMEQAKRDLQVEDYSISQTTLEQIFLQFTKYQQEAAIQ

SEQ ID NO: 5 - DNA, 5250 bp, Ostrinia nubilalis
atgaatcggaaaagaggaagcggaagggcggccggatcgtggatgaaattccgtttgctaatgtggaagaattttctgcaacaatggaggcatcctattcaaactatcgtagaggtgttgttgccagttatcacgatgtctctagtgctactgcttcggtggcagattgaacctaccgcagaagatgcgataaacttttcgccattacctgcacaatcactgaataattctatacaaatatttgctggtctcaacgtaactaatttgtccattgcatactctccaagaagtgaagccttggaggatgtcttacgtagttccatggcaaacttactgctaaacaatgcgttcgatctaatcagaattatcactgatctgtggcctgctgaaccgccattcccactgccagcgaatataacgtggcctgaaaccccgccagtcacactgcctccagatttagactggcctgatttagagggtgtaaacaaaactctgatttacgagtttctcagaaccttaatacgagttcaaccatacaattctagtgttgatctcagaactatttatgcttacgaagagagaactaagcaagtaatagcagctgtggaatttgatgactctctttttgatgctacggatttgcctggaagtatatcgtattccttaaggtttccggagaggcctcgccttaatgcattatttggtcgaggcggccgatcttggagaacagacgaattgtttcctgcatttgaattacctggaccaagatcccgttcttctgatggaggctccaatcctggctacgtcagggaaatgtttattgcgcttcagcaagtaatatcaacgcaactaataacaagaattactggggagcctatggagtcattctcagtcaatattcagagatatccacaccctgcatatgtcgatgatatggcagtagaggcactccagtttctatttcccatgttcattatgctgacctttagctacacagccgtgaatatcaccagagccatcacagttgagaaggaacttcaacttaaggaaacattgaaaattatgggcttacccacatggatacattggactgcatggttttgcaaacagttcctctttttattggtagcagctgttcttattatgattcttctaaaggttaattggtttacgaacgaagatgggttcagcgattacgccatatttacaaatacaccatggagtgtcttgctattctatttaacactctacttggcatgcgtgatattcttttgtttcatgctcagtggtatattttcgaaaggtagtacagcagcgctatttgccggtgtggtgtggttcctcacctacgtcccagcgtttatcctgtccattgacattgctatgtcagtccctatgcaaatatttacatgtctcagtatcaactctgcaatgtcttacggattccagctgcttctgggtagagagagcacaggcggtatgcaatggggcgagttcatgtcggcgcccgtgacggagaccgaccggctgctgttcgggcacgtggttatcatgctggtggtggactcggtgctgtacatgctgatcgcgctgtacctggagcaggtgctgcccggcccctacggcacgcccaggccctggtacttcttattccagaaacaattttgggggtgcggcactaaatctgcatcagattcagtattactaaatgttcctgaaactcctgatgtaataaaggagaacgatccggtgggtcacccagttggagttaaaatgaacaatctgacaaaagtattcggcaagaatactgcagtaaataatttgagtttaaatatatacgacgaccaaattacagttttgctcggccataatggagcaggaaaatctacaactatatcgatgttgacaggtaatttggaggcaacatatgggacagtgtgggtggccggctatgacatgacctggaacaccagtgatgctcgctcacatataggactatgccctcaacataacgtcctgttcaatgagctcaccgtcagggaacatttagaattctttgccagactgaaaggattccaaggcgaagaattgaacaatgaaatagacactcttatcgagaaactagagttgacagagaagcgtgactaccagtccaaaggcttgtccggcggacagaagcggaggctgtgcgttggcgtggcactgtgtggcgacgcgcgagtggtgctccttgacgagcctacgtcgggcatggacccctcctcgcgccgcgctctctgggacctattgcagaaggaaaaaaaaggtcgttccatgatattgacaacgcacttcatggacgaagctgacatcctgggggaccggatcgctattatggcgagcgggcagttgcaatgcgtcggctcgccatacttcctcaagcgacactacggcgtcgggtacactctggtcattgtcaaggatgttgacttccagttggaagcctgcacggagattttaagcaaatatattccaggaactactgttaaggaagataggggaacggaagttacatacaatttgactaacggtcgatctcaaatattcgaaacaatgttattggacctagaaacaaatatgcaacgaataaaattcaaaaactatggattagtagcaacgaccttagaggatgtgtttatgtcagttggatctgatgtatcatccacacagtcagagtcagatgaggtggaaactgcaactgagtcatcgtattacgactttgattcatcttcaatggataacttaacacaagaacagtcgatcagtggttttcgtttgttggtgcaacatgttttggccgtctggctgaagctgtctctggtctggatccggtcctggggcctgattctgttgcagattttggtaccggtgctacaaatgaccgcaacacttggagttatggaatacattttcagtttgataccaactatacagcgcagggcgctatcgtttgccgtaggttactcgcaaacggagagccttttgagtttcatcggcaattccacatcatcgctgggggcgctagcgactgcagcatacgagatgatgatcaactcgagcgtcgtcgacaccatgtcgattaccatggtcaatgaaccgattgatgaatattatatggaaagggcggaacaaggaggagtaggaccactgcgccacacggttttgtcgggtgccactttcacagatgattcggtaaccgcatggttcagcaacttcggctaccacgacattgccacttcgttagccgccgttcacaccgcgcttatcaaagctaaaaacgcttcgtatgaaatcaacgtgttcaaccatccactagaagtcaactatgctgatcaaagtgaccttcaaatgcgggtgacgatgctagccatgcaactggcttccggcatcgggagcagtttgggtattgccagtgcagttttcgtcatgttttatattaaggagcgcgtgtctcgagcgaaactgctgcaaaaggcggccggcatccagcctgcagttctatggggcagcgcagctgtgttcgactggctgtggttccttgtggtttgcgtcaccatcgtcatcatttgtgccgcttttgacgtcatgggcctctcatccgtcgacgaactcggtcgcctatacttgtgcctgatggtgtacggcgctgcgatggtgcctctgaactacctgtcttccctcctgttccaaggcccggcacttggcttcgttgtcatattcttcataaatgtactcttcggacttatgggggctcagatcgtggacgcactgtcatcgccgcagctaaataccgcagaggcggcgaggatcctggactatatactgcagttctttccactctacagtctcgtaacgtctgtcagactattgaatcagttcggcatgttagagagctcgtgcctacaaagctgtggatacttacagaacattatgaacataccagagtgcaatatgactgttatgtgcgatctctatgaagaatgctgtattcctgcagacccatactgggcctgggacggtggcattatgcgatatgtaactgtcatgctgatcacctgtcttgtattgtggggaatactcatgattatagagtacgacgttattaagaagataatcagacgagagaagaaaccaccacaagttgacgaaagcacactagacgaggatgtattggacgaagagaagcacgtcgcgcgcatcggctccgacctctctcagcacagcctcgtggcgcgcgggctctccaagtactacggcaagcacctggccgtcaatcaagtgtccttcagcgtaagcgactctgaatgcttcggtctgctgggggtgaacggagccggcaagaccaccacgttcaagatgctaatgggcgacgagaccgtctccagcggagacgcgttcgtcagtggccactctgtcaggaaggacatcacacgtgttcatgaaaatatcggatactgcccgcagttcgacgccgtattcggcgaactgacgggccgtgagacgttacttttgttctcgctgctccgcgggctcagcgccagccgcgcggacgctcgcacgcacgcgctggcgcatgcgctcggcttcaccaagcacttggacaaacgggtaaaccagtattcagggggcaacaaacgcaaattgagcacagcggtagccttactgggccgcactcggttggtgttcgtcgacgaacccaccactggcgtcgaccctgcggctaaacgacaggtatggcgtgctatccgaggcgcccagcgttcaggcagaggcgtggtgctgacgtcacatagcatggaggagtgcgaggcgctatgctcgcgcctcaccatcatggtcaacggccgcttccagtgccttggcacccctcaacacctgaaaaacaagttttctgaaggtttcactttaactattaaaatgagacaagaagaggaacaagcatcaactagttcatgcattgttcagagacctgtagatacggtcaaacaattcgtcgaatccaacttcactaacccaaaacttatggaagagtatcaaggtttgctgacatactacttgcccgaccgcagcatcgcatggtcaagaatgtttggaatcatggagcaagccaagagagacctccaagtggaggactacagcatttcccaaaccactttagaacagatcttcctacaatttaccaaatatcaacaagaggctgcaattcaataa

SEQ ID NO: 6 - protein, 1749 aa, Ostrinia nubilalis
MNRKRGSGRAAGSWMKFRLLMWKNFLQQWRHPIQTIVEVLLPVITMSLVLLLRWQIEPTAEDAINFSPLPAQSLNNSIQIFAGLNVTNLSIAYSPRSEALEDVLRSSMANLLLNNAFDLIRIITDLWPAEPPFPLPANITWPETPPVTLPPDLDWPDLEGVNKTLIYEFLRTLIRVQPYNSSVDLRTIYAYEERTKQVIAAVEFDDSLFDATDLPGSISYSLRFPERPRLNALFGRGGRSWRTDELFPAFELPGPRSRSSDGGSNPGYVREMFIALQQVISTQLITRITGEPMESFSVNIQRYPHPAYVDDMAVEALQFLFPMFIMLTFSYTAVNITRAITVEKELQLKETLKIMGLPTWIHWTAWFCKQFLFLLVAAVLIMILLKVNWFTNEDGFSDYAIFTNTPWSVLLFYLTLYLACVIFFCFMLSGIFSKGSTAALFAGVVWFLTYVPAFILSIDIAMSVPMQIFTCLSINSAMSYGFQLLLGRESTGGMQWGEFMSAPVTETDRLLFGHVVIMLVVDSVLYMLIALYLEQVLPGPYGTPRPWYFLFQKQFWGCGTKSASDSVLLNVPETPDVIKENDPVGHPVGVKMNNLTKVFGKNTAVNNLSLNIYDDQITVLLGHNGAGKSTTISMLTGNLEATYGTVWVAGYDMTWNTSDARSHIGLCPQHNVLFNELTVREHLEFFARLKGFQGEELNNEIDTLIEKLELTEKRDYQSKGLSGGQKRRLCVGVALCGDARVVLLDEPTSGMDPSSRRALWDLLQKEKKGRSMILTTHFMDEADILGDRIAIMASGQLQCVGSPYFLKRHYGVGYTLVIVKDVDFQLEACTEILSKYIPGTTVKEDRGTEVTYNLTNGRSQIFETMLLDLETNMQRIKFKNYGLVATTLEDVFMSVGSDVSSTQSESDEVETATESSYYDFDSSSMDNLTQEQSISGFRLLVQHVLAVWLKLSLVWIRSWGLILLQILVPVLQMTATLGVMEYIFSLIPTIQRRALSFAVGYSQTESLLSFIGNSTSSLGALATAAYEMMINSSVVDTMSITMVNEPIDEYYMERAEQGGVGPLRHTVLSGATFTDDSVTAWFSNFGYHDIATSLAAVHTALIKAKNASYEINVFNHPLEVNYADQSDLQMRVTMLAMQLASGIGSSLGIASAVFVMFYIKERVSRAKLLQKAAGIQPAVLWGSAAVFDWLWFLVVCVTIVIICAAFDVMGLSSVDELGRLYLCLMVYGAAMVPLNYLSSLLFQGPALGFVVIFFINVLFGLMGAQIVDALSSPQLNTAEAARILDYILQFFPLYSLVTSVRLLNQFGMLESSCLQSCGYLQNIMNIPECNMTVMCDLYEECCIPADPYWAWDGGIMRYVTVMLITCLVLWGILMIIEYDVIKKIIRREKKPPQVDESTLDEDVLDEEKHVARIGSDLSQHSLVARGLSKYYGKHLAVNQVSFSVSDSECFGLLGVNGAGKTTTFKMLMGDETVSSGDAFVSGHSVRKDITRVHENIGYCPQFDAVFGELTGRETLLLFSLLRGLSASRADARTHALAHALGFTKHLDKRVNQYSGGNKRKLSTAVALLGRTRLVFVDEPTTGVDPAAKRQVWRAIRGAQRSGRGVVLTSHSMEECEALCSRLTIMVNGRFQCLGTPQHLKNKFSEGFTLTIKMRQEEEQASTSSCIVQRPVDTVKQFVESNFTNPKLMEEYQGLLTYYLPDRSIAWSRMFGIMEQAKRDLQVEDYSISQTTLEQIFLQFTKYQQEAAIQ

SEQ ID NO: 7 - DNA, 5900 bp, Spodoptera frugiperda
agtgaatatgccgtactctattgttaagtgaaaacgttattcgttcaaatataaatcggtctaaaaatataaaaggaggcagacgccatcgccttaaaaatgcggctggtaccgaagcaggcgagccccttcgcaaagttccggctgctgatgtggaagaacttcctgcagcaatggaggcacagaacgcaaacagtcctcgaaattctactcccagttctcaccatgacgctggtgctaatcctacgatggcagatagaaccagcagaaagagaaacacaaacctatccgcctttcagagcaaacacgctcaacttttccactgttgtactgtttggtctggattgtcctaatgtatctattgcctattcaccaactagtcctgtgttagaagatgtagttagaaatgcaataactaatttattaatacagaacatggaagatttaattgctaggttaccaatagaaatagagttaccacccactattgaaataaattctaccgctatacttgactggataaaatctcgtataagggtacaagcttataataatagtcatgaaacaagagggatttatatagaagaagaaaatacacgaagggttatagcggtcgtagagtttgacgataaactatacggcgcagaatcattgtccaataatttgtcgtactctctgagattccccgagagacctcgtcttaattcgttattccaaactggaggtcgcacttggaggacagacgcagttttccctgttttcgaaacgcctggtccacgattttcgaagtcttgggaaggtggtaacgatccaggttacgtaaacgaaatgttcatagcactgcaacaagttatttcaacggaattgatatcgaggtccacaggagtgaacatgagtgagttcacagtgttcctgcagaggtatcctcacccgccatacatcagggacatggcgcttgaccttctgcagttcatgttccctatgttcatcatgttgagcttcagttacactgccatcaacattacaagggcggtcacggtggaaaaggaattgcagctaaaggaaacgatgaaaatcatgggtctccctacatggttgcattggactgcatggacttgtaaacagtttgttttcttgttagtatccgcaagtctgacagtgatacttttaaaaataaattggttcacaaacgaagacggtttcagtgagtacacggtgtttactaacacgccttggacagtcttgatgtttttcacgatgctgtacttaacttgcgtgatatttttctcatttatgatgagcagctttttctctaaagctagtacagcagcgttgtgcactgtagttgtctggttcctcacatacatccctgccttcctcctggcaatggacatcgaaatgtctacaacagtccaggtctttacatgcctcagtattaattctgcgatgtcttatggattccagcttttgcttgccaaggaaagtactggaggcttgcagtggggcgacttcatgtcggcaccagggacggactctaatcgcttcgtgttcggccacgtggtcatcatgttagtggtggactgcttcgtctacatgctcgtcaccctctacttcgaacaagtgatgcctggaccattcggcacgcccaaacgctggtacttcccgttccagttgcgattttggttccctcattataaaccaggtacagttttggttttggaaaatgaaaacagtgaatttgaagacatcataaaggaaaaagaacccaatgaacacgaagtgggcgttaaaatgaataatttaacaaaaatctttggagctaatacggcggtcaacaacttatctttaaatatttacgacgatcaaatcacagtattacttggacataatggtgctgggaaatcgaccacaatatcaatgttgacaggcaatttggaggtaacccggggtacagtgactgtcgcgggatatgacatgacacacgaaacttatgcggctcgtgcccatatcggcttatgtccccaacacaatgtactgtttaacgaactcactgtcagagaacatttggaattctttgccaggttgaaaggatttaggggagcagagctcaagtctgagattgatactcttatcgaaaagttggaactacaagacaagcgagactatccctctaatggtttgtctggaggtcaaaagcgacgtttatgtgttggaatagctttaagtggagcagcacgtgtagtactactcgatgaacctacttctggaatggatccttcttcccgacgagctctttgggaacttctgcagaaggagaagaaaggtcgatcgatgatcttgacaacgcatttcatggacgaagctgatattctcggtgatcgcgtggcaattatggcgcaaggccgcttgcagtgtgtgggttcaccttatttcctgaaacgtcattatggcgtcggatacactctggtagtcgttaaggacgatgactttgatttcgaggaatgcacaagacttataaataaatacattccagacacggttgtcaaggaagaccgcggaacggaaattacttataacttaatcaacgattattcatacgcttttgaagaaatgctgaacgatttggaatgtaatatggaaaagataaaatacaaaaactatggtttaactgctaccactttagaggatgtgtttatgtcagtcggttcggacttggcaccagtaaataattcagataacgatgacgctgttactactacgactgactcgaccattgatgacattttgaaacatgaacttgattcgtctttggaagaattggatagagacgaaagcagtgtgaccggtttccgattgctatgtcagcaagtattagcagtgtggatgaagaagggactgacactgattcgctcaccttggttgatgatcttgcagttcttcgctcccgtcattctcatcaatgccacgcttggagttatgagatacgtgatgtccttaaccccaactataagatctagatttttatcattgactgaaggattcacgagtacggagaccttgttaagtttcaacggtacattaggatcgtctgtcggcgctatcgcggcagcagcatacgagatgatattcacggcctcagacgtggaaaacatgggcgtgactcacattgggaacgtacccatggacgagtattatctaaataggacgatggatccagtggtgatggggcagttacgtcaccagatcctgattggttccactttcgacgataacaatgcgacactttggttcagtaacttcggttaccacgacgtcgctatagctctatctaccttccactcagcttttcttagagcattcaactctactgcgcagcttaacgtttacaatcatccattggaggctacttatagagaccagacggacatgcagatgatgattgccatgctctctatgcagctgtcttctggtattggtagtagcgtcagtatcgtcagtgctgtattcatcatgttctttattaaggagcgcacatctggcgctaagttgttacaaaaggccgcgggagtacaaccagcagtgctttggggtagtgctgctgtcttcaactgggcttggttcctgattacctgcgtttccatagtaatcacttgtgccgctttccaagttattggactgtccactgctcaagaattagctcgaatgtacttgtgcgtaatgttgtacggtgctgcaatgttgccgctggtgtacatcctgtcgtttgcgttcaatgggcctgccgttggcttcgtcggttattactttatgaacgtgctttttggtatgatgggtgcacaaatagtggaagcactgtcttctcctcagctgaacacagcggaagccgcgaatatactcgactacatattacagttcttcccgctttacagtttaatcactgcagtcagatttttgaaccaagttgggctgcgtgaatacacttgtctacaaatgtgcgagtactatcaagccgtgaaccctaaccttcagtgcaccatggaaagcttatgttcacgctacgaagaatgctgtgtcgagccgaatgtttatttcaaatggaatcagccgggcgtatcgcgatacttgacaagtatgattatatcctgcatcgtgttctggacgattcttatgatcatcgagtacagggtcttccagaagttatgtacgatcaagaagaccccaccacctctagacgagagtatactggacgaggacgtgcaaaaagaggcgcagcgcgcgcgcaacgtgttgccctcgcaacgctacgagcatgcgctgatcgccaacgatctctccaagtactacggaaaacatcttgccgtaaatcaaatctcatttggcgttaacgacggcgaatgtttcggtctattgggtgtaaacggtgccggtaagactaccaccttcaagatgttaatgggtgacgagtctatctcaagcggcgaagcgtttgttagtggacactctgtcgagaaaagtcttggcaaagtacaccagaatatcggttactgtccccagttcgatgctttgtttggtgagctcacgggccgtgagacgctacatatgtttgctatgatgaagggtctgcgtttacgcagtgctgcaccaaccgctgaaacattagcacatgcactcggtttcctcaaacatcttgacaaaagggtaaatcaatattcaggaggaacgaaaagaaagctaaacacggcgatagcattcttgggacgaacgcgtcttgtgttcgtggatgagcccaccactggagtagatcctgccgctaagcgacatgtatggcgagcaacgcgtggcgtacagcgcgctggccgcggcgtagtgctgacgtcacacagcatggaggagtgcgaagctctttgctcacggctcactatcatggtcaacggacggttccagtgtttcggcacaccgcaacatcttaagaacaaattttctgaaggtttcacgttaattattaagatgaaaatggaagacagagacaacgacactgcttcgataaacagctcacgtagtgtagtggacactgtcaaagaatatgttactcaaaacttccagaatccacgtatcatggaggaataccagggactcctgacgtactaccttccagaccgtagtatggcgtggtcacggatgttcggcattatggagcgtgctaagagggacttggagattgaagactacagcatctcacagactacactagaacaaatattcttacagtttacaaagtaccagcgacaagcattcgaattactatagtgaacagtttcgtagtttagaggtaattcgttacatatcccacattggggatctgataattcggggacatgctctctggtaatattatgcgtgagtgcatagtttcatatattagatacaaacagtgcaatgccacttgtgtactagtatgacatcacagatgagtctcgctctagtcgataaaattgtagcaaaaattgttactaacattttatggtatagctctaagaagttaaagtgctaaattaaaaaataattaggtacactggcttctgtttgtatacgagaaacatttttccatataatcgtcttatatttgggctggaaacccgaaatatttataaacctagttggataaattaattaagtagtcaaagtagtagtttagttatcataaatgtcgtctcaacaactacttgtacattgtagttagaggagaggtctgaaggcaagcgtcaacagggtcgcatcgtacgcattccgtatgacgtcattagtacgcaatgcatgtaggcattgctgatgatgcggtccgtacgatgcgtcggccggtgttgtatgaatttctatacaagacaaactaaaatccgttg

SEQ ID NO: 8 - protein, 1735 aa, Spodoptera frugiperda
MRLVPKQASPFAKFRLLMWKNFLQQWRHRTQTVLEILLPVLTMTLVLILRWQIEPAERETQTYPPFRANTLNFSTVVLFGLDCPNVSIAYSPTSPVLEDVVRNAITNLLIQNMEDLIARLPIEIELPPTIEINSTAILDWIKSRIRVQAYNNSHETRGIYIEEENTRRVIAVVEFDDKLYGAESLSNNLSYSLRFPERPRLNSLFQTGGRTWRTDAVFPVFETPGPRFSKSWEGGNDPGYVNEMFIALQQVISTELISRSTGVNMSEFTVFLQRYPHPPYIRDMALDLLQFMFPMFIMLSFSYTAINITRAVTVEKELQLKETMKIMGLPTWLHWTAWTCKQFVFLLVSASLTVILLKINWFTNEDGFSEYTVFTNTPWTVLMFFTMLYLTCVIFFSFMMSSFFSKASTAALCTVVVWFLTYIPAFLLAMDIEMSTTVQVFTCLSINSAMSYGFQLLLAKESTGGLQWGDFMSAPGTDSNRFVFGHVVIMLVVDCFVYMLVTLYFEQVMPGPFGTPKRWYFPFQLRFWFPHYKPGTVLVLENENSEFEDIIKEKEPNEHEVGVKMNNLTKIFGANTAVNNLSLNIYDDQITVLLGHNGAGKSTTISMLTGNLEVTRGTVTVAGYDMTHETYAARAHIGLCPQHNVLFNELTVREHLEFFARLKGFRGAELKSEIDTLIEKLELQDKRDYPSNGLSGGQKRRLCVGIALSGAARVVLLDEPTSGMDPSSRRALWELLQKEKKGRSMILTTHFMDEADILGDRVAIMAQGRLQCVGSPYFLKRHYGVGYTLVVVKDDDFDFEECTRLINKYIPDTVVKEDRGTEITYNLINDYSYAFEEMLNDLECNMEKIKYKNYGLTATTLEDVFMSVGSDLAPVNNSDNDDAVTTTTDSTIDDILKHELDSSLEELDRDESSVTGFRLLCQQVLAVWMKKGLTLIRSPWLMILQFFAPVILINATLGVMRYVMSLTPTIRSRFLSLTEGFTSTETLLSFNGTLGSSVGAIAAAAYEMIFTASDVENMGVTHIGNVPMDEYYLNRTMDPVVMGQLRHQILIGSTFDDNNATLWFSNFGYHDVAIALSTFHSAFLRAFNSTAQLNVYNHPLEATYRDQTDMQMMIAMLSMQLSSGIGSSVSIVSAVFIMFFIKERTSGAKLLQKAAGVQPAVLWGSAAVFNWAWFLITCVSIVITCAAFQVIGLSTAQELARMYLCVMLYGAAMLPLVYILSFAFNGPAVGFVGYYFMNVLFGMMGAQIVEALSSPQLNTAEAANILDYILQFFPLYSLITAVRFLNQVGLREYTCLQMCEYYQAVNPNLQCTMESLCSRYEECCVEPNVYFKWNQPGVSRYLTSMIISCIVFWTILMIIEYRVFQKLCTIKKTPPPLDESILDEDVQKEAQRARNVLPSQRYEHALIANDLSKYYGKHLAVNQISFGVNDGECFGLLGVNGAGKTTTFKMLMGDESISSGEAFVSGHSVEKSLGKVHQNIGYCPQFDALFGELTGRETLHMFAMMKGLRLRSAAPTAETLAHALGFLKHLDKRVNQYSGGTKRKLNTAIAFLGRTRLVFVDEPTTGVDPAAKRHVWRATRGVQRAGRGVVLTSHSMEECEALCSRLTIMVNGRFQCFGTPQHLKNKFSEGFTLIIKMKMEDRDNDTASINSSRSVVDTVKEYVTQNFQNPRIMEEYQGLLTYYLPDRSMAWSRMFGIMERAKRDLEIEDYSISQTTLEQIFLQFTKYQRQAFELL

SEQ ID NO: 9 - DNA, 5283 bp, Chrysodeixis includens
atgcatgctcgagcggccgccagtgtgatggatatctgcagaattcggcttatgaagatgaggcgagaggctaagcccgcgggcgcgttcatgaagttccgcctgctgatgtggaagaacttcctgcagcagtggcggcaccggctgcagacggtgacggagctgctgctgcccgtgctcaccatgacgctggtgctggtgctgcgctggcagatggagcccagcatggtcggcaccctcacgtacccgccgataccagcacacacactcaactattctacagccattttagcgggtatgaacttaacacagatgtccatagcatactcaccaagaagtccagtattagatgacgtggtcagaactggaattacaaacttattagttgcgaatgcaaaagatctgcttccaatttttgaaaatatttcaatacctggtttaccagaaatagaacttccatctattcctgaagatttcaactctacactaattgtagagttcttgaagtctcgaataaaaatcgaggcttacaacaatagtcatgatctaagaggactgtacatccgcgaggagtccactcgcgtggttatcgctgggattgagtttgatgacaaactttatgatgcggaaagcttgtcaaacaatttatcatttgcactacggtttcctgaaagacctcgaatgaactctttcttccaacgaggggggcgcacttggcgaacagatatagtgttcccgttatttgaaatgccaggaccaagatatccatggtcatgggaaggcggcagagatccaggctacgtcaacgaaatgtttatcgcgctgcaaaacgctatctctaatgagttgatatccagggctaccggggaggacttgaaaaagttcagggttaatgtccagagattccctcacccgccttacatactcgacatggcggtggatttactgcagttcatgttcccgatgttcatcatgctgagcttcagttacactgccgttgatattgccagagcagtcacagtagaaaaagaattgcaactgaaggaaacgatgaagatcatgggcctacccacgtggctacactggacagcgtggttttgcaagcagtttctctatcttcttattacagcgattttaattatagttcttttaaagatacattggtttactaacgaagagggcttttctgaatacgcagtgtttactaataccccgtggacggtgctgttctttttcatggttttgtacttatcatgcgctatatttttctgttttatgataagcagtttcttttcaaaaggtagtacagcggctttgtgcatgggagtggtctggttcctgtcttacgtccccgccgttctcttggccatggacatcgacatgtctactgcaatgcaagtcttcacgtgccttagcatagattcagcgatgtcttacggtttccaacttctactcgccaaagaagccgttggaggtttgcagtggggcgacttcatgtcgtcaccggcggcggagacgaaccgcttcgtgttcgggcacgtggtcatcatgctggtggtcgactgtgtgctgtacatgctcgtcactctgtacctggagcaggtcatgccagggccctttggcacgcccaaaccctggtatttccccttccaaatgaaattttggtttcctaattatagttcagatgtcggttttattttggaaaacgaagttagtgagtctgaagatataattaaagagaaagacccaatcgaccatacaatcggtgtcaaaatgcatgatctaacaaaaatttacggaaataatgtagcggtcaatcatttatctttgaatatttacaacgatcagattaccgtgctcttggggcacaatggtgcggggaaatctaccaccatatctatgttgacaggaaatttaaaggcaactcgcgggtctatgagcgtggctgggtacgacatgagctcgcaagccgcggccgctcgcgcacacatcggcttgtgtccacaacacaacgtcttgttcaacgaactcaccgtcaaggaacatctccaatttttcgcccgcctcaaaggattcaaaggccaacaattgaaagacgaaattgatactcttatcgctaaattggagttggaagaaaagcgtgattatccttctaaaggtctttctggcggtcagaagcgtcggttatgcgttggtatagctttaagcggggcagctcgtgtcgtgctcctggacgaaccaacctcgggcatggaccctgcatcccgacgagctctctgggaccttttgcaacgggagaagaaaggtcgctcaatgatcccgacgacccactttatggacgaggcagacattctcggcgacagagtggccattatggcgaacggtcgcctgcagtgcgtgggctccccgtacttcctcaagcgtcactatggcgtcgggtacaccctggtcgttgtcaaggatgatgacttcgacttcgaggaatgctctaagctgattcataaatatgtcccaggcagtattatgaaggaagatcgcggttccgaaatcgcgtatagtcttgacaatgattactctcacactttcgaaaatatgttaaatgatcttgaaaagaatattggcacaattaaactgaaaaattatggcttggttgcaactactctggaagatgtatttatgtctgttggcgcggacctggcacctgtacagtcagagtcggacgacactgccaccacaaccaccgactcctcaatggacgatatactcaaacatgaaatcgattcttctttagaacaattggatagagacgagagcagcgtcaaaggtttcagtctattgtaccagcacgttctagcggtgtggatgaagttggccttagtctggatccgatcttggtggctggtgctattgcagtttgccgcccctgtagtcctgataaacgccacgcttggagtcttgcaatacgttatgtcgttcgcgcctatcattacaagcagggttttagatcttacagaaggttatgttctcaccgagaccctgttgagctacaacggttcttcgtcgacgtcgctcggagctctcgcagcacaagcctatgaaacgatgttcaaaacctccggagtcaacagtatggagctcacgttgattggcagtcgaccagtcgaagattattatctagagagggcaaacgacacagtggcaatggctaacctacgtcaccgcctgctgatcggctccactttcgatgaaaactcagctacggcccggttcagtaactttggctaccacgacgtagccacgtcgctagcaactgtctactcagctatactcaaagccaagaactcaaccgcttttatgaacgtttacaatcatcccctggaagccacgtattcggatcaaagtgacttgcagacgatgatagcgatgttgtccatgcagctggcgtctggcatcggcagcagtgtgggcatcgtgagcgctgtgttcatcatgttctatatcaaggagcgtgtatcgggcgccaagttgctgcagaaggcggcgggcgtgcagccggccgtgctgtggggcgcggccgctgtgttcgactggacgtgcttcttactcacctgcatatctatagtcatctcctgtgcggccttccaggtcataggcctgtctacagcttccgagttgggccgcatgtacctgtgtgcgatggtgtacggcgcggcgatgttgccgttcagctacatcatgtcgcacgtgttcagaggaccagccgtcggcttcgttagcttcttcttcatgaatgtcatctttggtatgatgggcccgcaagtggtggaggcgctgtcttcgccgacgctcaccacgcagcacgtggcgcacattatggacaacgtgctgcagttcttcccgctctacagtcttgttacatcagtcaggtatttgaaccagatcggcctccgtgagtacacgtgcctgcaaagctgtgaatacttgcaggcggtgtaccctaacgtcgagtgcacgatggccagcatgtgcgaattctccagtaactgttgcgttcgagataacccgtacttcgactgggaggagcctggcgttctgcggtacttggtcgccatgacaggcacctgcgccgttctatggacgattctgatggtcatcgagtacagactcttccaaaaggtattaagattccgcaagaccccgccgccagtggacgagagctcgttagacgaggacgtggcgcgcgaggctgagagcgcgcgtcacacgcactacgccgaccgcgcgaaccatgccctcctcgccacggacctcgccaagtactacgggaaacatctcgcggtggaccaagtgtccttcagtgtaagcgacggcgaatgtttcggtctgctgggcgtgaacggcgcgggcaagaccacgaccttcaagatgctgatgggcgacgagtccatctccagcggcgaggcctacgtcagcggacactctgtgcggaagaacctgaacagggtgcatgagaatattggttactgtccgcaattcgacgcgttattcggcgagctgactggtcgcgagacgctccgcatgttcgccctgatgcgaggacttcgcctcagcacggccgcgcccgctgtggagacgctctcacacgcactcggcttcttaagacatctcgataagagggtggatcaatattcaggaggcactaagcgaaagctgaacacagcgatagcgttcttaggaaagacgagacttgtgttcgtcgacgaacctaccaccggcgtagaccctgctgctaaacgacatgtatggcgagccacgcggggcgtgcagcgcgcaggccgtggcgtggtgctgacgtcacacagcatggaggagtgcgaagcgctgtgctcacgactcaccatcatggtcaacggacggttccaatgccttggaacaccacaacatctcaagaataaattctctgaaggttttacgttgactatcaaaatgaaaatggaggataatcctgaaacatcgtcgaacagcagcgcaatcagtaaagtggaccttgtcaaggaatacgtcgaagctaatttccagactcctaggataatggaggaataccaaggtctactaacatactacctaccagaccggacaatggcgtggtcgcgaatgttcggtatcatggagcgagcgaaacgagacttagaaatcgaggattacagcatatcacagacgacattagaacagatattcctacaattcacaaagtaccagcgacaagaaggcgatgaatcatag

SEQ ID NO: 10 - protein, 1760 aa, Chrysodeixis includens
MHARAAASVMDICRIRLMKMRREAKPAGAFMKFRLLMWKNFLQQWRHRLQTVTELLLPVLTMTLVLVLRWQMEPSMVGTLTYPPIPAHTLNYSTAILAGMNLTQMSIAYSPRSPVLDDVVRTGITNLLVANAKDLLPIFENISIPGLPEIELPSIPEDFNSTLIVEFLKSRIKIEAYNNSHDLRGLYIREESTRVVIAGIEFDDKLYDAESLSNNLSFALRFPERPRMNSFFQRGGRTWRTDIVFPLFEMPGPRYPWSWEGGRDPGYVNEMFIALQNAISNELISRATGEDLKKFRVNVQRFPHPPYILDMAVDLLQFMFPMFIMLSFSYTAVDIARAVTVEKELQLKETMKIMGLPTWLHWTAWFCKQFLYLLITAILIIVLLKIHWFTNEEGFSEYAVFTNTPWTVLFFFMVLYLSCAIFFCFMISSFFSKGSTAALCMGVVWFLSYVPAVLLAMDIDMSTAMQVFTCLSIDSAMSYGFQLLLAKEAVGGLQWGDFMSSPAAETNRFVFGHVVIMLVVDCVLYMLVTLYLEQVMPGPFGTPKPWYFPFQMKFWFPNYSSDVGFILENEVSESEDIIKEKDPIDHTIGVKMHDLTKIYGNNVAVNHLSLNIYNDQITVLLGHNGAGKSTTISMLTGNLKATRGSMSVAGYDMSSQAAAARAHIGLCPQHNVLFNELTVKEHLQFFARLKGFKGQQLKDEIDTLIAKLELEEKRDYPSKGLSGGQKRRLCVGIALSGAARVVLLDEPTSGMDPASRRALWDLLQREKKGRSMIPTTHFMDEADILGDRVAIMANGRLQCVGSPYFLKRHYGVGYTLVVVKDDDFDFEECSKLIHKYVPGSIMKEDRGSEIAYSLDNDYSHTFENMLNDLEKNIGTIKLKNYGLVATTLEDVFMSVGADLAPVQSESDDTATTTTDSSMDDILKHEIDSSLEQLDRDESSVKGFSLLYQHVLAVWMKLALVWIRSWWLVLLQFAAPVVLINATLGVLQYVMSFAPIITSRVLDLTEGYVLTETLLSYNGSSSTSLGALAAQAYETMFKTSGVNSMELTLIGSRPVEDYYLERANDTVAMANLRHRLLIGSTFDENSATARFSNFGYHDVATSLATVYSAILKAKNSTAFMNVYNHPLEATYSDQSDLQTMIAMLSMQLASGIGSSVGIVSAVFIMFYIKERVSGAKLLQKAAGVQPAVLWGAAAVFDWTCFLLTCISIVISCAAFQVIGLSTASELGRMYLCAMVYGAAMLPFSYIMSHVFRGPAVGFVSFFFMNVIFGMMGPQVVEALSSPTLTTQHVAHIMDNVLQFFPLYSLVTSVRYLNQIGLREYTCLQSCEYLQAVYPNVECTMASMCEFSSNCCVRDNPYFDWEEPGVLRYLVAMTGTCAVLWTILMVIEYRLFQKVLRFRKTPPPVDESSLDEDVAREAESARHTHYADRANHALLATDLAKYYGKHLAVDQVSFSVSDGECFGLLGVNGAGKTTTFKMLMGDESISSGEAYVSGHSVRKNLNRVHENIGYCPQFDALFGELTGRETLRMFALMRGLRLSTAAPAVETLSHALGFLRHLDKRVDQYSGGTKRKLNTAIAFLGKTRLVFVDEPTTGVDPAAKRHVWRATRGVQRAGRGVVLTSHSMEECEALCSRLTIMVNGRFQCLGTPQHLKNKFSEGFTLTIKMKMEDNPETSSNSSAISKVDLVKEYVEANFQTPRIMEEYQGLLTYYLPDRTMAWSRMFGIMERAKRDLEIEDYSISQTTLEQIFLQFTKYQRQEGDES

SEQ ID NO: 11 - DNA, 5208 bp, Spodoptera frugiperda
atgcggctggtaccgaagcaggcgagccccttcgcaaagttccggctgctgatgtggaagaacttcctgcagcaatggaggcacagaacgcaaacagtcctcgaaattctactcccagttctcaccatgacgctggtgctaatcctacgatggcagatagaaccagcagaaagagaaacacaaacctatccgcctttcagagcaaacacgctcaacttttccactgttgtactgtttggtctggattgtcctaatgtatctattgcctattcaccaactagtcctgtgttagaagatgtagttagaaatgcaataactaatttattaatacagaacatggaagatttaattgctaggttaccaatagaaatagagttaccacccactattgaaataaattctaccgctatacttgactggataaaatctcgtataagggtacaagcttataataatagtcatgaaacaagagggatttatatagaagaagaaaatacacgaagggttatagcggtcgtagagtttgacgataaactatacggcgcagaatcattgtccaataatttgtcgtactctctgagattccccgagagacctcgtcttaattcgttattccaaactggaggtcgcacttggaggacagacgcagttttccctgttttcgaaacgcctggtccacgattttcgaagtcttgggaaggtggtaacgatccaggttacgtaaacgaaatgttcatagcactgcaacaagttatttcaacggaattgatatcgaggtccacaggagtgaacatgagtgagttcacagtgttcctgcagaggtatcctcacccgccatacatcagggacatggcgcttgaccttctgcagttcatgttccctatgttcatcatgttgagcttcagttacactgccatcaacattacaagggcggtcacggtggaaaaggaattgcagctaaaggaaacgatgaaaatcatgggtctccctacatggttgcattggactgcatggacttgtaaacagtttgttttcttgttagtatccgcaagtctgacagtgatacttttaaaaataaattggttcacaaacgaagacggtttcagtgagtacacggtgtttactaacacgccttggacagtcttgatgtttttcacgatgctgtacttaacttgcgtgatatttttctcatttatgatgagcagctttttctctaaagctagtacagcagcgttgtgcactgtagttgtctggttcctcacatacatccctgccttcctcctggcaatggacatcgaaatgtctacaacagtccaggtctttacatgcctcagtattaattctgcgatgtcttatggattccagcttttgcttgccaaggaaagtactggaggcttgcagtggggcgacttcatgtcggcaccagggacggactctaatcgcttcgtgttcggccacgtggtcatcatgttagtggtggactgcttcgtctacatgctcgtcaccctctacttcgaacaagtgatgcctggaccattcggcacgcccaaacgctggtacttcccgttccagttgcgattttggttccctcattataaaccaggtacagttttggttttggaaaatgaaaacagtgaatttgaagacatcataaaggaaaaagaacccaatgaacacgaagtgggcgttaaaatgaataatttaacaaaaatctttggagctaatacggcggtcaacaacttatctttaaatatttacgacgatcaaatcacagtattacttggacataatggtgctgggaaatcgaccacaatatcaatgttgacaggcaatttggaggtaacccggggtacagtgactgtcgcgggatatgacatgacacacgaaacttatgcggctcgtgcccatatcggcttatgtccccaacacaatgtactgtttaacgaactcactgtcagagaacatttggaattctttgccaggttgaaaggatttaggggagcagagctcaagtctgagattgatactcttatcgaaaagttggaactacaagacaagcgagactatccctctaatggtttgtctggaggtcaaaagcgacgtttatgtgttggaatagctttaagtggagcagcacgtgtagtactactcgatgaacctacttctggaatggatccttcttcccgacgagctctttgggaacttctgcagaaggagaagaaaggtcgatcgatgatcttgacaacgcatttcatggacgaagctgatattctcggtgatcgcgtggcaattatggcgcaaggccgcttgcagtgtgtgggttcaccttatttcctgaaacgtcattatggcgtcggatacactctggtagtcgttaaggacgatgactttgatttcgaggaatgcacaagacttataaataaatacattccagacacggttgtcaaggaagaccgcggaacggaaattacttataacttaatcaacgattattcatacgcttttgaagaaatgctgaacgatttggaatgtaatatggaaaagataaaatacaaaaactatggtttaactgctaccactttagaggatgtgtttatgtcagtcggttcggacttggcaccagtaaataattcagataacgatgacgctgttactactacgactgactcgaccattgatgacattttgaaacatgaacttgattcgtctttggaagaattggatagagacgaaagcagtgtgaccggtttccgattgctatgtcagcaagtattagcagtgtggatgaagaagggactgacactgattcgctcaccttggttgatgatcttgcagttcttcgctcccgtcattctcatcaatgccacgcttggagttatgagatacgtgatgtccttaaccccaactataagatctagatttttatcattgactgaaggattcacgagtacggagaccttgttaagtttcaacggtacattaggatcgtctgtcggcgctatcgcggcagcagcatacgagatgatattcacggcctcagacgtggaaaacatgggcgtgactcacattgggaacgtacccatggacgagtattatctaaataggacgatggatccagtggtgatggggcagttacgtcaccagatcctgattggttccactttcgacgataacaatgcgacactttggttcagtaacttcggttaccacgacgtcgctatagctctatctaccttccactcagcttttcttagagcattcaactctactgcgcagcttaacgtttacaatcatccattggaggctacttatagagaccagacggacatgcagatgatgattgccatgctctctatgcagctgtcttctggtattggtagtagcgtcagtatcgtcagtgctgtattcatcatgttctttattaaggagcgcacatctggcgctaagttgttacaaaaggccgcgggagtacaaccagcagtgctttggggtagtgctgctgtcttcaactgggcttggttcctgattacctgcgtttccatagtaatcacttgtgccgctttccaagttattggactgtccactgctcaagaattagctcgaatgtacttgtgcgtaatgttgtacggtgctgcaatgttgccgctggtgtacatcctgtcgtttgcgttcaatgggcctgccgttggcttcgtcggttattactttatgaacgtgctttttggtatgatgggtgcacaaatagtggaagcactgtcttctcctcagctgaacacagcggaagccgcgaatatactcgactacatattacagttcttcccgctttacagtttaatcactgcagtcagatttttgaaccaagttgggctgcgtgaatacacttgtctacaaatgtgcgagtactatcaagccgtgaaccctaaccttcagtgcaccatggaaagcttatgttcacgctacgaagaatgctgtgtcgagccgaatgtttatttcaaatggaatcagccgggcgtatcgcgatacttgacaagtatgattatatcctgcatcgtgttctggacgattcttatgatcatcgagtacagggtcttccagaagttatgtacgatcaagaagaccccaccacctctagacgagagtatactggacgaggacgtgcaaaaagaggcgcagcgcgcgcgcaacgtgttgccctcgcaacgctacgagcatgcgctgatcgccaacgatctctccaagtactacggaaaacatcttgccgtaaatcaaatctcatttggcgttaacgacggcgaatgtttcggtctattgggtgtaaacggtgccggtaagactaccaccttcaagatgttaatgggtgacgagtctatctcaagcggcgaagcgtttgttagtggacactctgtcgagaaaagtcttggcaaagtacaccagaatatcggttactgtccccagttcgatgctttgtttggtgagctcacgggccgtgagacgctacatatgtttgctatgatgaagggtctgcgtttacgcagtgctgcaccaaccgctgaaacattagcacatgcactcggtttcctcaaacatcttgacaaaagggtaaatcaatattcaggaggaacgaaaagaaagctaaacacggcgatagcattcttgggacgaacgcgtcttgtgttcgtggatgagcccaccactggagtagatcctgccgctaagcgacatgtatggcgagcaacgcgtggcgtacagcgcgctggccgcggcgtagtgctgacgtcacacagcatggaggagtgcgaagctctttgctcacggctcactatcatggtcaacggacggttccagtgtttcggcacaccgcaacatcttaagaacaaattttctgaaggtttcacgttaattattaagatgaaaatggaagacagagacaacgacactgcttcgataaacagctcacgtagtgtagtggacactgtcaaagaatatgttactcaaaacttccagaatccacgtatcatggaggaataccagggactcctgacgtactaccttccagaccgtagtatggcgtggtcacggatgttcggcattatggagcgtgctaagagggacttggagattgaagactacagcatctcacagactacactagaacaaatattcttacagtttacaaagtaccagcgacaagcattcgaattactatag

SEQ ID NO: 12 - protein, 1249 aa, Helicoverpa zea
MRLETRHASAATKFRLLMWKNFLQQWRHRLQTVVELLLPVVTMALVLILRWQIPPYQIDTLTYPALPAHTLNYSTNILFAMNMEELSIAYSPASPVLDDVMRTAVINLLTANMKDLIPIFIDNLPPGIANITFPPDMNLNTSAIEEFVKSRIRVVPYNSSYEIRGIYVDEETTRSIIAAVEFDDKLYGAEQLSNNLSYSLRFPERPRLNSFFQTGGRTWRSDGVFPVFEVPGPRFPHSWEGGNDPGYVNEMFVALQQVISMELVSRATGLDLKSFRVNIQRYPHPPYLHDQSVDLLQFMFPLFIMLSFSYTAVNIARAVTVEKELQLKETMKIMGLPTWLHWTAWFVKQFIYLSITAVLLVVLLKVNWFTNDDGFSEYAVFTNTPWTVLLFFLILYLSCAIFFSFMVSSIFSKGSTAALFMAVAWFLTYIPAFLLAMDINMSTAVQVITCFSINSAMSYGFQLMLAKESTGGLQWGDFMTSPGTDTTRFVFGHVVIMLVVDCLIYMLITLYLEQVLPGPFGTPKPWYFPFQLQFWFPNYKSKDAGLIFENDNSEFDDIIKEKDPTDHEVGVKMQNLTKIFGNNIAVNNLSLNIYDDQITVLLGHNGAGKSTTISMLTGNLKVTRGTVNVAGYDMTSQSSAARSHIGLCPQHNILFNELTVKEHLEFFARLKGFKGKELYEEIDSLIEKLELQEKRDYPSKGLSGGQKRRLCVGIALSGAARVVLLDEPTSGMDPSSRRALWELLQKEKKGRSMILTTHFMDEADILGDRVAIMANGRLQCVGSPYFLKRHYGVGYTLVIVKDTDFDFVKCSVLINSYIPGTIVKEDRGTEITYNLVNDYSHVFEEMLNDLERNIDNIKFKNYGLVATTLEDVFMSVGADLSPINSESDDAITTTTDSTIDDILKQEIDSSLEELDKDESNVTGLRLFGQQVLAVWMKQWLVLIRSPWVMVLQFLAPVVLINSTLGVLRYVMSLSPTIRTRWLSLEEGYTESETLLSFNGSVASSVGALAAQAYQSLFANSGVMDMEINAIGSQPIEEYYLNRTSDPVVMGSLRHRLLIGSTFDDNSATAWFSNFGYHDVATSLAAIHSAILRSKNSDAVLNVYNHPLEASYIDQSDVQTMIAMLSMQLSSGIGSSVSIVSAVFIMFYIKERMSGAKLLQNAAGVAPSVLWGGAAIFNWFWFLITCVSIVISCVAFDVIGLSNVHELGRMFLCVMVYGAAMLPLVYLLSLKFKGPAVGFVGFYFLNVLFG

SEQ ID NO: 13 - DNA, 11229 bp, Helicoverpa zea
atgagattagaaacgaggcacgctagtgccgccaccaagttccgcctgctcatgtggaagaactttctgcagcaatggagacatcggctacaaactgtcgtagagctactattgccagtcgtgacaatggcgctggtgctcatccttcggtggcagataccgccttatcaaatcgatacactcacttaccccgcgttgccagcgcacacactcaactactctaccaatatcctgtaagttaccacaaaatattcttcttttaaattgctaatatccaacgtttttcatctttggttggatgacaaccaatttcatcaactcgtatgcttatttatcttaatccatcctaaaaaatctgtcaagcttcttgcccggtaatcaatatttaaggcaatcagattcagttgtgtgcctttaaatattacagatattgtaaagatagcgcatttatagttatagctaggtcctactaacttgctccactataaacattttataacataaacaaacatttattcacgaacattttattcacagacagcattcgttttatatattttctaaaataatttctagagaggtatattcaggataggatgtgtgtttatcaaataatctttgaagttcatacatatcctttatcatatctaaaagctaattcttttgctaaacatactgtaaaattataagtggtaatctgtatgtttagtcaattttggtgactaagcaatagataaaacagagatgttttattgattaggtacagttaaagattgccgaagttacaatcgcggtaatttatgaaagcatcaaataaacataaggattcgatgatttcctcagaacattaaatgcaaatattaggtacctacttgtcgccttcaatcattgcagattttcatttctagagactatagctgctgaaagttttctttaaatattgctacaataaatacatttcaaatatgagaatttccgtttattattcaaagttatgaaatttaaataatatacaatgagcatctgtattattaaaactcatagttactcttatgtctatgcactctaggtatatgttttcattcgtgaacacatttgctatttgtactcactccataagccgagataaggcgataatattacatctcgtggttataatcgtccctaatgtttaatgaaattcatttttcaggtttgccatgaatatggaagaattatcaattgcatactccccggcaagtccagtgttagatgatgtaatgagaactgctgttattaatttactaacagccaatatgaaagatctgattcctatttttattgataacttaccaccggggatagccaatataacatttccaccagatatgaacttaaatacgtcggccattgaggagttcgtgaagtcacgaatacgagtcgtaccttataacagcagttatgaaataagagggatctacgttgacgaagaaactacacgcagcattatcgctgccgtcgagtttgacgataaactatatggtatgaataaactcattgcttacacaattttttgtgcctatttccagctattgatattcaggtaatgttacaaacagcggcttctaatctatttcaggagcagaacagttgtcaaataatttatcttattcgctacgttttcctgagagaccccgtctcaattcgtttttccaaactggagggcgcacctggagatctgacggagtattcccggttttcgaagtgcctgggcctagatttcctcactcgtgggaaggtggtaatgacccaggtaaatatatttattcgctttaggacgtctggcaaacactaattaaaaaatatattaactttgtttaaaataaaaacaagtaaggtatcgaaactaacgaaaataaggtattgttaggattaggtacatactgaaattaatcaagtattgtgttaactacaataaatattgtgcacaggttacgttaacgaaatgttcgtggcacttcaacaagttatttccatggagctggtatcaagggcaaccgggttggacttgaagtcatttagggtgaacatacagaggtacccgcacccgccgtaccttcacgaccagtcagtggatctgctgcaatttatgttccccctgttcatcatgttgagcttcagctacactgccgtcaacattgcacgggcggtcacagttgagaaggaattgcaattaaaggtacctagatagtaattccaaggtacaatgttggaggataccaattaaataaccttgctaatttttattatgttgttttaggaaactatgaaaattatgggcctccccacatggttgcactggacagcatggtttgttaaacagtttatctacctatcaatcacagctgttctgctagttgtgttgctaaaggtgagattttcaaaattagtacacagattcttacgtagtttagatttaataataacttatgtattgtactgtacttattgtttttaggtaaattggtttactaacgacgatggcttcagcgaatatgctgtatttactaatacaccttggacggttttgctattcttcttgatactgtatttatcttgcgcgatatttttttctttcatggtaagcagtatattttcaaaaggtatgctttcttcttatttctttttaatttaacaacctacattaacatatttaacaatatattatttttgtggtaaaggtagtacggccgcgttgtttatggcggtggcatggttcctcacttacatccctgctttcctcctggccatggatatcaatatgtcgactgcggtgcaggtcatcacatgcttcagtattaactctgcgatgtcctatggtttccaactaatgctcgctaaggaaagcactggaggtaaattattacgagagtttattatacgagccacttaaaataattacaatataaatgctctgctccaaagtttgtttgatccgaatcagggaatagaaaaattatgcccaataattataattttcctaatttgttgcaccgcttcacacgtaacacgattcgaacaagacggagtgcgagtggcggtaattgaagtcgatgtgccacattagcgtaggagaataaattctaacatagcgactggtctaagtgctaaattgctttactgaacgtattcgctcaattagggctgcagtggggcgacttcatgacgtcaccagggacggacaccacgcgcttcgtgttcggccacgtggtcatcatgctggtagtggactgcctcatctacatgctgatcaccctctaccttgaacaagtgctaccaggccccttcgggactcccaaaccttggtacttccccttccagttgcagttctggttcccaaattataaatcgaaaggtaagatattttgtagagttgtactctacctaatttatgaatcaaattagtaatttaaaatagtcattgttacaagcattaatgtcatagcaataatcattatgctacaaagtcgaattaggtatctttgtaataaaatccaatgactcaaaaatgctatgattttttttagatgctggattaattttcgaaaatgataatagtgaattcgatgatattataaaagaaaaggatcccacagaccacgaagttggtgttaaaatgcaagtaagtttatcatcacttaaaagtaacattaagtacttacacactaatatattatgtgaaaatatatttgttaattcattaaaaaaaatatttgtacgtatttatttacagaatttaacaaaaatctttgggaataacatagctgttaacaatttatctttgaatatctatgacgaccaaatcacagttttacttggtcataatggtgctggcaaatcaactacaatatctatgttaacaggtttgtaataaaatattttttatttatgctcttctttaaccatgtttttaaatataattaacaaggattaagtgcttgtatttagtttgtacccgggagcttcgttgattttcaatgacattattagatggagattagggataagagtactttttgatagccttgtcactatctatgtctatatcctccgtatgcaaagttgcgataaaatgcaattcattcaagttttacttaaaatactaggatgagtgaagatgtaacatacgttgcagagtgggtgcaagtcaattgattcactactatgaagtcttgtttctggtaataagcaaagaccggaactattgcgcaacattgttattgtcaagtgggcactagctttaatgtaacgatgccggtcagtgtgttaatctatctaaatagagttgaaggttcttcaaacatttttctgtctaacgacgaacacacattatatgtaatttcagcggcacgcggaaaccattttataaaagagtatgaattgtcccagttattatggtccgtttttaccacccatttaaaaaaaaaagcagttgacgactcgtattgtccgaggttttacatgggcggaaagatgtaaataaaatcgaaatcatatacatatgtcataacatactttaatttttgaatatattgtgtttcaggaaatttaaaggtaactcgcgggacagtgaacgttgccggatatgatatgacttctcaaagctccgcagcccgttcccacattggattgtgtcctcagcacaatatactgtttaacgaactcacggtcaaagaacatttggaattctttgctagactaaaaggatttaaaggcaaagaactgtatgaagagatagactcacttattgaaaaattggaactacaggagaaggtatctacaatttatgattatttggaatttgtcagttcagttctgtctctatcaattagagagatttttttaattagagcggcctcatatcatatttcttcaagttacaaatggaactagaaagtgattgggcgtacctgcaatataagacatgttatttgtttcacagcgtgactacccctcaaaaggtctgtcaggaggtcagaagcgtcgtctttgtgttggtattgctctgagtggggcggcacgagtggtcttactcgacgaacctacgtctggcatggatccttcatctcgtcgagcactgtgggaactcttacagaaggaaaagaaaggtaggtcgaaattcaatcgttagaacagagaattaggtatttctttgtataaagtatttttgttttaaattattattaatataaagggaccgacatataaacctaatagtgatggctatagtcgcatgttaatatgtttattatatcgttaggtcgctcgatgatcctgacgactcattttatggacgaagcagatattctcggcgacagagtggctataatggcaaacggtagactgcaatgcgtgggctctccgtatttcctcaaacgtcattatggcgtcgggtataccctggtgatcgttaaggacacagacttcgactttgtgaaatgctccgtacttatcaatagctatattcctggtactattgttaaagaagatcgaggtaagtttttactggttaaaatgttgtctgatatatgatccatcaaactataattgcgtccttttattgagaatactagcttttgcccgcggcttcgctcccgtcaactgacttctctactttaccctatttttttttcataagaaccttctcctgacaataacaaacacaacaaaaaaagaattagccaaattggtccaggttgagttatgcgcttaccaacacattttgcgattcatttttatattatagattaaatataaacaaatatttcttttttaaggaacggaaatcacttataatttggtaaatgattactcacacgtttttgaagaaatgttgaatgatttggaaagaaatattgataacatcaaatttaaaaactatggtttagttgctactacattagaagatgtctttatgtcgtaagtattttctataaatgtttagttgttagtacgttagcctaattttggtaagtggggtgataacaaagtacttatgttgcagcgttggtgcagacttaagtccaattaattccgaatctgacgatgctattactactactactgactcgactatcgatgatatattaaaacaagaaatcgattcatctttggaagaacgtaagttgtccaaagatattgggtattttaaacgaaaaaaaaaaaaatcattagatatcctttttacaacttgattacaaaaacggtccttgttttctagtggataaggacgagagtaacgtgacgggtctccgcttgttcggtcagcaagtgctggctgtatggatgaagcagtggctggtgctgatccgctcgccatgggtcatggtactgcagtttttggcgccagtggtactcatcaactccacgctaggagttctgcgttacgtcatgtctttatcaccgaccattagaactaggtggttgtcgttggaagaagggtgagttaaatagtaaaatcactttattgacatcggaaggcgcaacgcctattgaccggttaagttcgtgaaattcttttatctattatatcatcaagtaaatctaggtacttatattgggaatcgatcgtatcgacgcctaagtagtatctgctaggacataaaatacttacctacatattgcttgacatcaacaatctacctaaatgcaataggactcttgtgtcagagactgcgaatcttatggaacttacagtacctaaatacataacatacctacatacatataacatcataaattattttcaggtatatggaaagcgaaactctgctcagcttcaacggcagtatagcgtcatcagtgggtgccctagccgcgcaagcataccaaagcctgttcgccaattctggtgttatggacatggaaatcaacgctatcggaagccagccaatagaagaatattatctaaatagagtgagtcactatacaatttcgaagtcggcaaccgatatatcatgatttcgataaagacataaacagggatcaaaacttcgataaataaataatcacctgctatatcataattacaagtcattatgagtgagattcatacattattgtcatgatggatggatggatttacatttttacaaatttaaatgaactcatccataaaaaaaaaactcaatccttaggctataaatactcaatcagattgcactgaaacggagagctgtggagtttttaaacatctttcgttaggtattttcgttatactgaaatatcctttctccatccactcagtaaatctattggttatataaaaacctctaacttgtaggtacttgctaataagagattttttgcttacacagacaagtgatcccgttgtgatgggttcgctgcggcaccgcttgctgataggctccacatttgacgacaactctgctaccgcctggttcagtaactttggctaccacgatgttgctacatcgcttgcggcaatccactcagctattctcagatctaaaaactctgatgcagtactcaatgtatataatcatccgttggaagcttcgtatatagatcaggtaagttttacagacgaactgtctatatctttgtactctttctttttaacctatacagcttagtcgtagcaattagaattaatcgctaatgtcttcaatgaatgttttcagagtgacgtgcagactatgatagctatgttgtccatgcagttgtcctctggcatcggcagtagtgtgagcattgttagtgcggttttcatcatgttttatatcaaggtaagtacattatttaacctcgaaaagtattttctgctcttatgtaccgattctttcaaagttttgtgacttcttcaggttataacggttcgtttttgttttcgcaggaacgtatgtcgggggcaaaacttctacaaaatgcagcaggcgtggcgccttctgtgctgtggggcggcgcagcgatcttcaattggttttggttcctcatcacttgtgtttccatcgtcatctcgtgcgtcgcttttgatgtcatcgggttatcgaacgtgcatgaattaggttatatggtatttcctagtagcagtccttttaaataatgtttaaaagaaactgattaaatgtgacttgtacctttataggctggtactaattaataattgcaatttttatttcaggtcgaatgtttttgtgcgtcatggtatacggtgcggcgatgttgccattagtgtaccttttgtcgcttaagttcaagggaccagctgtcggcttcgtgggcttctatttcctcaacgtgcttttcggtaagttgtaggatagccaaaagggttcaagtatatatataagtattgagtattgtatacttaaattaagtttcagagtcacccataatgaatctctgctttctttttccctttttgtagtgaccgtattattactgaatcgggtaggttcctgaaaattcacttgtgttgtattctacaggtatgatgggtgcgcaggtggtggaggcactatcctctcctatgctggacacagagcaagccgcccacatccttgactacttactgcagttctacccgctttacagtcttgtcacttctatcaggtaggtactcgctgcaacaagcccaatatcctacgttctaaaccatataagttaaagtaataacacattatattttcatttgggtggtgatctcaatccgtataatttcgatttgcaggtttttaaatcaggtcggcctacgggagtatacttgcttacaaggctgtgaatacttgcaggcagtatacccgaatctagagtgtagcatggcaagcatgtgcgaattccacagtaactgctgcggtaagtggcatagcttcccataaatatgaattaattgagatgataataggtatcatcaatttatttgtcgatttctccttgcagttcgtgaaaacccatacttcgattgggaggaaccaggcgtcctgaggtacttgctcagtatgtgcttctcctgcctaatcttctggttgctgcttatgaccattgaatacagagtggtgcaaaaggtaattatagccctgtatttacatcattaaaaattattagtccattgagatattttctaaaattaaaactgtaaccattttttgacacatttctatacacttttggtcaaagtttaaaaaatgtttctgacgtttggtttcgatgtgtcgtaggtgttcacattcaagaagactcctcctccaatagacgagagcacgttagacgaggacgtgatgacagaggcgaggcgcgcgcgccaggtgccgccgacacgccgcagcgaccacgcgcttctcgctcacgacctctccaagtactacgggaaacatctcgccgtagaccaagtctcgttcagtgagtcaacttcgcatttaaaaaaataatttgtattgtaggtacatttatatacattgtccggaatttcgaaattaacttttccattattcatcaaggtgtgaacgacggcgaatgcttcggtctattgggtgtgaatggtgccggaaaaacgaccaccttcaagatgctgatgggtgatgagtccatttcaagcggcgaggcgtatgtctccgggcactcggtgcagaggaatctcgatagagtacacgagaatattggtcagtagccaaaaaacctcttgaaattttaatataaaaacttttattaagtactgctgacacgtaggttcgtgtaaccacgtacatatattcatgttatctaatttatattgcaggatattgtccgcaatttgacgcattatttggtgagctgacgggtcgccagacactacacatgtttgcgttgatgcgcggcttgcgtttacgcactgcagcaccttcggctgaaacactcgcacatgcgcttggcttcttcaaacatcttgataaaagggtaatcttttatttctttaaatatatgtctaggcctactgtattgtagttgtcataacatttattttgaatttaatttttcaggtgcatcagtattcaggcggcacgaaacgcaagcttaacacggcgatagcattcatgggacgaacacggcttgtgtttgttgatgagcctaccactggagttgatcccgccgctaaacgccacgtgagtacttcgacctaatcagcgaaaaccagaacaagtagactcaagccatctttcgataggatatagtgcatgccctcgagtggtcttaaggatactagatttaggttttttttggtccttaacattcagcaatatagaatccaacgtttttaaattgatcaggtatggcgcgctacccgcggcgtgcagcgagcaggtcgcggcgtggtgctgacgtcacatagtatggaggagtgcgaggcgttgtgttcgcgactgaccatcatggtcaacggtcgcttccagtgtctgggaacgccacaacatctcaagaacaaattttctcaaggtaaaggaaaaaatcgatagtgtcgttcatcggattcattcgtgttttgcataaccgaatgtagagcaggcccctaccaatgttttacataggtctgtctaataagaaaacccctttgtaagtttaatggtataccagtcgttgatttacaaattacaccctgtgacaaagtctttttgcgttttcccagtcacgcagagacctactgtcattaaaagatggtcactcattctcccattcacaccaggacagaccgcgttaacggcttattgataaataacttgtttcaggctttactttaatcattaaaatgaaaactgacgacagtgaaagcgacacgcagtcagtaaacagcactaccagcgtagtagatagtgtcaaactatacgtctctgggaactttgaaagtccaaagataatgtaagtaacaaaatctatttcaagttctggagcatagaattagaacttaaaaactgaatttatcactatcgtttcaattagaattccagcagtggtgtgctttattggggtaattggctgctctttatatagataattctatttttcagggaagagtatcatggtcttctaacttactacttgcctgaccgtagcatggcatggtcacgaatgtttggtatcatggagcgcgccaaacagatcttacaaattgaggactacagcatatcgcagactaccctcgaacaaatattcttgcagttcaccaaataccaaagagaagaaggaacgacgttataa

SEQ ID NO: 14 - DNA, 12949 bp, Helicoverpa zea
atgagattagaaacgaggcacgctagtgccgccaccaagttccgcctgctcatgtggaagaactttctgcagcaatggagacatcggctacaaactgtcgtagagctactattgccagtcgtgacaatggcgctggtgctcatccttcggtggcagataccgccttatcaaatcgatacactcacttaccccgcgttgccagcgcacacactcaactactctaccaatatcctgtaagttaccacaaaatattcttcttttaaattgctaatatccaacgtttttcatctttggttggatgacaaccaatttcatcaactcgtatgcttatttatcttaatccaccctaaaaaatctggcaagcttcttgcccggtaatcaataaggcaatcagattcagttgtgtgcctttaaatattacagatattgtaaagatagcgcatttatagttatagctaggtcctactaacttgctccactataaacattttataacataaacaaacatttattcacgaacattttattcacagacagcattcgttttatatattttctaaaataatttctagagaggtatattcaggataggatgtgtgtttatcaaataatctttgaagttcatacatatcctttatcatatctaaaagctaattcttttgctaaacatactgtaaaattataagtggtaatctgtatgtttagtcaattttggtgactaagcaatagataaaacagagatgttttattgattaggtacagttaaagattgccgaagttacaatcgcggtaatttatgaaagcatcaaataaacataaggattcgatgatttcctcagaacattaaatgcaaatataaaggtagaattccgtgggtcgcggcttacgcagccgcgcgcggcttacgacagtcgtcggccgcgcgcggctgtcgtagccgctatccacggaattctacctttaggtacctacttgtcgccttcaatcattgcagattttcatttctagagactatagctgctgaaagttttctttaaatattgctacaataaatacatttcaaatatgagaatttccgtttattattcaaagttatgaaatttaaataatatacaatgagcatctgtattattaaaactcatagttactcttatgtctatgcactctaggtatatgttttcattcgtgaacacatttgctatttgtactcactccataagccgagataaggcgataatattacatctcgtggttataatcgtccctaatgtttaatgaaattcatttttcagttttgccatgaatatggaagaattatcaattgcatactccccggcaagtccagtgttagatgatgtaatgagaactgctgttattaatttactaacagccaatatgaaagatctgattcctatttttattgataacttaccaccggggatagccaatataacatttccaccagatatgaacttaaatacgtcggccattgaggagttcgtgaagtcacgaatacgagtcgtaccttataacagcagttatgaaataagagggatctacgttgacgaagaaactacacgcagcattatcgctgccgtcgagtttgacgataaactatatggtatgaataaactcgattgcttacacaattttttgtgcctatttccagctactgatattcaggtaatgttacaaacagcggcttctaatctatttcaggagcagaacagttgtcaaataatttatcttattcgctacgttttcctgagagaccccgtctcaattcgtttttccaaactggagggcgcacctggagatctgacggagtattcccggttttcgaagtgcctgggcctagatttcctcactcgtgggaaggtggtaatgacccaggtaaatatatttattcgctttaggacgtctggcaaacactaattaaaaaatatattaactttgtttaaaataaaaacaagtaaggtatcgaaactaacgaaaataaggtattgttaggattaggtacatactgaaattaatcaagtattgtgttaactacaataaatattgtgcacaggttacgttaacgaaatgttcgtggcacttcaacaagttatttccatggagctggtatcaagggcaaccgggttggacttgaagtcatttagggtgaacatacagaggtacccgcacccgccgtaccttcacgaccagtcagtggatctgctgcaatttatgttccccctgttcatcatgttgagcttcagctacactgctgtcaacattgcacgggcggtcacagttgagaaggaattgcaattaaaggtacctagatagtaattccaaggtacaatgttggaggataccaattaaataaccttgctaatttttattatgttgttttaggaaactatgaaaattatgggcctccccacatggttgcactggacagcatggtttgttaaacagtttatctacctatcaatcacagctgttctgctagttgtgttgctaaaggtgagattttcaaaattagtacacagattcttacgtagtttagatttaataataacttatgtattgtactgtacttattgtttttaggtaaattggtttactaacgacgatggcttcagcgaatatgctgtatttactaatacaccttggacggttttgctattcttcttgatactgtatttatcttgcgcgatatttttttctttcatggtaagcagtatattttcaaaaggtatgctttcttcttatttctttttaatttaacaacctacattaacatatttaacaatatattatttttgtggtaaaggtagtacggccgcgttgtttatggcggtggcatggttcctcacttacatccctgctttcctcctggccatggatatcaatatgtcgactgcggtgcaggtcatcacatgcttcagtattaactctgcgatgtcctatggtttccaactaatgctcgctaaggaaagcactggaggtaaattattacgagagtttattatacgagccacttaaaataattacaatataaatgctctgctccaaagtttgtttgatccgaatcagggaatagaaaaattatgcccaataattataattttcctaatttgttgcaccgcttcacacgtaacacgattcgaacaagacggagtgcgagtggcggtaattgaagtcgatgtgccacattagcgtaggagaataaattctaacatagcgactggtctaagtgctaaattgctttactgaacgtattcgctcaattagggctgcagtggggcgacttcatgacgtcaccagggacggacaccacgcgcttcgtgttcggccacgtggtcatcatgctggtagtggactgcctcatctacatgctgatcaccctctaccttgaacaagtgctaccaggccccttcgggactcccaaaccttggtacttccccttccagttgcagttctggttcccaaattataaatcgaaaggtaagatattttgtagagttgtactctacctaatttatgaatcaaattagtaatttaaaatagtcattgttacaagcattaatgtcatagcaataatcattatgctacaaagtcgaattaggtatctttgtaataaaatccaatgactcagaaatgctatgattttttttagatgctggattaattttcgaaaatgataatagtgaattcgatgatattataaaagaaaaggatcccacagaccacgaagttggtgttaaaatgcaagtaagtttatcatcacttaaaagtaacattaagtacttacacactaatatattatgtgaaaatatatttgttaattcattaaaaaaaatatttgtacgtatttatttacagaatttaacaaaaatctttgggaataacatagctgttaacaatttatctttgaatatctatgacgaccaaatcacagttttacttggtcataatggtgctggcaaatcaactacaatatctatgttaacaggtttgtaataaaatattttttatttatgctcttctttaaccatgtttttaaatttaattaacaaggattaagtgcttgtatttagtttgtacccgggagcttcgttgattttcaatgacattattagatggagattagggataagagtactttttgatgccttgtcactatctatgtctatatcctccgtatgcaaagttgcgataaaatgcaattcattcaagttttacttaaaatactaggatgagtgaagatgtaacatacgttgcagagtgggtgcaagtcaattgattcactactatgaagtcttgtttctggtaataagcaaagaccggaactattgcgcaacattgttattgtcaagtgggcactagctttaatgtaacgatgccggtcagtgtgttaatctatctaaatagagttgaaggttcttcaaacatttttctgtctaacgacgaacacacattatatgtaatttcagcggcacgcggaaaccattttataaaagagtatgaattgtcccagttattatggtccgtttttaccacccatttaaaaaaaaaagcagttgacgactcgtattgtccgaggttttacatgggcggaaagatgtaaataaaatcgaaatcatatacatatgtcataacatactttaatttttgaatattttgtgtttcaggaaatttaaaggtaactcgcgggacagtgaacgttgccggatatgatatgacttctcaaagctccgcagcccgttcccacattggattgtgtcctcagcacaatatactgtttaacgaactcacggtcaaagaacatttggaattctttgctagactaaaaggatttaaaggcaaagaactgtatgaagagatagactcacttattgaaaaattggaactacaggagaaggtatctacaatttatgattatttggaatttgtcagttcagttctgtctctatcaattagagagatttttttaattagagcggcctcatatcgtatttcttcaagttacaaatggaactagaaagtgattgggcgtacctgcaatataagacatgttatttgtttcacagcgtgactacccctcaaaaggtctgtcaggaggtcagaagcgtcgtctttgtgttggtattgctctgagtggggcggcacgagtggtcttactcgacgaacctacgtctggcatggatccttcatctcgtcgagcactgtgggaactcttacagaaggaaaagaaaggtaggtcgaaattcaatcgttagaacagagaattaggtatttctttgtataaagtatttttgttttaaattattattaatataaagggaccgacatataaacctaatagtgatggctatagtcgcatgttaatatgtttattatatcgttaggtcgctcgatgatcctgacgactcattttatggacgaagcagatattctcggcgacagagtggctataatggcaaacggtagactgcaatgcgtgggctctccgtatttcctcaaacgtcattatggcgtcgggtataccctggtgatcgttaaggacacagacttcgactttgtgaaatgctccgtacttatcaatagctatattcctggtactattgttaaagaagatcgaggtaagtttttactggttaaaatgttgtctgatatatgatccatcaaactataattgcgtccttttattgagaatactagcttttgcccgcggcttcgctcccgtcaactgacttctctactttaccctattttttttttcataagaaccttctcctgacaataacaaacacaacaaaaaaagaattagccaaattggtccaggttgagttatgcgcttaccaacacattttgcgattcatttttatattatagattaaatataaacaaatatttcttttttaaggaacggaaatcacttataatttggtaaacgattactcacacgtttttgaagaaatgttgaatgatttggaaagaaatattgataacatcaaatttaaaaactatggtttagttgctactacattagaagatgtctttatgtcgtaagtattttctataaatgtttagttgttagtacgttagcctaattttggtaagtggggtgataacaaagtacttatgttgcagcgttggtgcagacttaagtccaattaattccgaatctgacgatgctattactactactactgactcgactatcgatgatatattaaaacaagaaatcgattcatctttggaagaacgtaagttgtccaaagatattgggtattttaaacgaaaaaaaaaacattagatatcctttttacaacttgattacaaaaacggtccttgttttctagtggataaggacgagagtaacgtgacgggcctccgcttgttcggtcagcaagtgctggctgtatggatgaagcagtggctggtgctgatccgctcgccatgggtcatggtactgcagtttttggcgccagtggtactcatcaactccacgctaggagttctgcgttacgtcatgtctttatcaccgaccattagaactaggtggttgtcgttggaagaagggtgagttaaatagtaaaatcactttattgacatcggaaggcgcaacgcctattgaccggttaagttcgtgaaattcttttatctattatatcatcaagtaaatctaggtacttatattgggaatcgatcgtatcgacgcctagtatctgctaggacataaaatacttacctacatattgcttgacatcaacaatctacctaaatgcaataggactcttgtgtcagagactgcgaatcttatggaacttacagtacctaaatacataacatacctacatacatataacatcataaattattttcaggtatatggaaagcgaaactctgctcagcttcaacggcagtatagcgtcatcagtgggtgccctagccgcgcaagcataccaaagcctgttcgccaattctggtgttacggacatggaaatcaacgctatcggaagccagccaatagaagaatattatctaaatagagtgagtcactatacaatttcgaagtcggcaaccgatatatcatgatttcgataaagacaaacagggatcaaaacttcgataaataaataatcacctgctatatcataattacaagtcattatgagtgagattcatacattattgtcatgatggatggatggatttacatttttacaaatttaaatgaactcatccataaaaaaaaatactcaatccttaggctataaatactataaatactcaatcagattccactgaaacggagagctgtggagtttttaaacatctttcgttaggtattttagttatactgaaatatcctttctccatccactcagtaaatctattggttatataaaaacctctaacttgtaggtacttgctaataagagattttttgcttacacagacaagtgatcccgttgtgatgggttcgctgcggcaccgcttgctgataggctccacatttgacgacaactctgctaccgcctggttcagtaactttggctaccacgatgttgctacatcgcttgcggcaatccactcagctattctcagatctaaaaactctgatgcagtactcaatgtatataatcatccgttggaagcttcgtatatagatcaggtaagttttacagacgaactgtctatatctttttactctttctttttaacctatacagcttagtcgcaattagaattaatcgctaatctcttcaatgaatgttttcagagtgacgtgcagactatgatagctatgttgtccatgcagttgtcctctggcatcggcagtagtgtgagcattgttagtgcggttttcatcatgttttatatcaaggtaagtacattatttaacctcgaaaagtattttctgctcttatgtaccgattctttcaaacttttgtgacttcttcaggttataacggttcgtttttgttttcgcaggaacgtatgtcgggggcaaaacttctacaaaatgcagcaggcgtggcgccttctgtgctgtggggcggcgcagcgatcttcaattggttttggttcctcatcacttgtgtttccatcgtcatctcgtgcgtcgcttttgatgtcatcgggttatcgaacgtgcatgaattaggtaatatggtgtttcctagcagtgccggttttaactctaccagcgtcctgggcgagatttctacggcgccctccaactgcaactcaagcccatacgaaaaactgagacatatgtagttatcaatttgcgcgagcgaagcgagcgcgaattctgtttttatagttaacaagtcaaagcaaaaattaagtaaaatgtagcccaatcgtacctacataagttattgctggttggtgaatgcaaaaacatgggtacatggcttctgattgcgcgagcgaagcaagcgcgaaattatttgttatattttagaactcaaaacaaaaattacttgactctttgttggtgttggcgcctgtgtataaaaattttgggacttaaagtagtaccgtaaataagaaagtccatggaaactagaagttactttcttattaataaaagaacttaaaaacgacgctacctttttgctagggtagactaaaaaattgttgaaaaataaaaacacctgtaaagtgaagcaagcccgaaaatttttgagttttggatcactaaaagtcctaaacatatcatcatcctccgagcctttttcccaatcatgttggggtcggcttccagtctaaccggattcagctgagtaccagtgctttacaagaagcgactgcctatctgacctcctcaacccagttacccgggcaacccgataccccttggttagactggtgtcagacttactggcttctgactacccgtaacgactgccaaggatgttctatgacagccgggacctacagtcctaaaagtcctaaacatatgtatacaggaattaattttaagcagtgcgcaaaaaaaaactggtggtccggaatcattaacagaacatatctgcatcttgagtaaaacaaaatttttcattcttttgtccgccctaaaatcagcaaaatatggataccaactattcttacgcgcttggagcagcgctcgcgtcagttaaccggtttcgctttgcagcgcgcctactataacgactgtgactgtacaatcggttacaaaataaacatctttcgatatcaataacttttcttttttgtactaaaacacgacaaaacaaaaatatacgtatctataaaacttacttaattataggttgacaaagtttgcgcactggataaaattcattcctgtataataaaaggcgactgtgaagtgaagaagccgcgagcgaagggagcgcgaattttttgagtattgggacattaaaagtagctgtatgtcataaaaaaaacaaagtgtaaagtaaagaaatcgcgagcgaagcgagcgcgaaaatttttgagttttgggacatcaaagtagtgtttgttcgagaatttctcggaattaaacacaaattcgcttcggcgcccctgagcccgcagcgcccgggttattcgcacaccctgcaccataagttaagacggccctgatgctatccatacatttggatacagttcttgtaagtttcgatctttgatgaaatttaaaacaaaaagaagagtaacattctgatcaaggattggttgggggcgcctgtggcgcccccttatgaccggcgccctgggccgtcgcccaaccgcgccctaccctaaagccgctactgtttcctagtagcagtccttttaaataatgtttaaaagaaactgattaaatgtgacttgtacctttataggctggtactaattaataattgcaattattatttcaggtcgaatgtttttgtgcgtcatggtatacggtgcggcgatgttgccattagtgtaccttttgtcgcttaagttcaagggaccagctgtcggcttcgtgggcttctatttcctcaacgtgcttttcggtaagttataggatagccaaaagggttcaagtatatatataagtattgagtattgtatactaaaattaagtttcagagtcacccataatgaatctctgctttctttttccctttttgtaatgaccgtattattcctgaatcgggtaggttcctgaaaattcacttgtgttgtattctacaggtatgatgggtgcgcaggtggtggaggcactatcctctcctatgctggacacagagcaagccgcccacatccttgactacttactgcagttctacccgctttacagtcttgtcacttctatcaggtaggtactcgctgcaacaagcccaatatcctacgttctaaaccatataagttaaagtaatcacacattatattttcatttgggtggtgatctcaatccgtataatttcgatttgcaggtttttaaatcaagtcggcctacgggagtatacttgcttacaaggctgtgaatacttgcaggcagtatacccgaatctagagtgtagcatggcaagcatgtgcgaattccacagtaactgctgcggtaagtggcatagcttcccataaatatgaattaattgagatgataataggtatcatcaatttatttgtcgatttctccttgcagttcgtgaaaacccatacttcgattgggaggaaccaggcgtcctgaggtacttgctcagtatgtgcttctcctgcctaatcttctggttgctgcttatgaccattgaatacagagtggtgcaaaaggtaattatagccctgtatctacatcattaaaaattattagtccattgaggtattttctaaaattaaaactgtaactattttttgacacatttctatacacttttggtcaaagtttaaaagatgtttctgacgtttggtttcgatgtgtcgtaggtgttcacattcaagaagactcctcctccaatagacgagagcacgttagacgaggacgtgatgacagaggcgaggcgcgcgcgccaggtgccgccgacacgccgcagcgaccacgcgcttctcgctcacgacctctccaagtactacgggaaacatctcgccgtagaccaagtctcgtttagtgagtcaacttcgcatttaaaaaaataatttgtattgtaggtacatttatatacattgtccggaatttccaaattaacctttccattattcatcaaggtgtgaacgacggcgaatgcttcggtctattgggtgtgaatggtgccggaaaaacgaccaccttcaagatgctgatgggtgatgagtccatttcaagcggcgaggcgtatgtctccgggcactcggtgcagaggaatctcgatagagtacacgagaatattggtcagtagccaaaaaacctcttgaaattttaatataaaaacttttattactgctgacacgtaggttcgtgtaaccacgtacatatattcatgttatctaatttatattgcaggatattgtccgcaatttgacgcattatttggtgagctgacgggtcgccagacactacacatgtttgcgttgatgcgcggcttgcgtttacgcactgcagcaccttcggctgaaacactcgcacatgcgcttggcttcttcaaacatcttgataaaagggtaatcttttatttctttaaatatatgtctaggcctactgtattgtagttgtcataacatttattttgaatttaatttttcaggtgcatcagtattcaggcggcacgaaacgcaagcttaacacggcgatagcattcatgggacgaacacggcttgtgtttgttgatgagcctaccactggagttgatcccgccgctaaacgccacgtgagtacttcgacctaatcagcgaaaaccagaacaagtagactcaagccatctttcgataggatatagtgcatgccctcgagtggtcttaaggatactagattcaggtttttttttggtccttaacattcagcaatatagaatccaacgtttttaaattgatcaggtatggcgcgctacccgcggcgtgcagcgagcaggtcgcggcgtggtgctgacgtcacatagtatggaggagtgcgaggcgttgtgttcgcgactgaccatcatggtcaacggtcgcttccagtgtctgggaacgccacaacatctcaagaacaaattttctcaaggtaaaggaaaaaatcgatagtgtcgttcatcggattcattcgtgttttgcataaccgaatgtagagcaggcccctaccaatgttttacataggtctgtctaataagaaaacccctttgtaagtttaatggtataccagtcgttgatttacaaatgacaccctgtgacaaagtctttttgcgttttcccagtcacgcagagacctactgtcattaaaagatggtcacccattctcccattcacaccaggacagagcgcgttaacggcttattgataaataactttaatttgtttcaggctttactttaatcattaaaatgaaaactgacgacagtgacagcgacacgcagtcagtaaacagcactaccagcgtagtagatagtgtcaaactatacgtctctgggaactttgaaagtccaaagataatgtaagtaacaaaatctatttcaagttctggagcatagaattagaacttaaaaactgaatttatcactatcgtttcaattagaattccagcagtggtgtgctttattggggtaattggctgctctttatatagataattctatttttcagggaagagtatcatggtcttctaacttactacttgcctgaccgtagcatggcatggtcacgaatgtttggtatcatggagcgcgccaaacagatcttacaaattgaggactacagcatatcgcagactaccctcgaacaaatattcttgcagttcaccaaataccaaagagaagaaggaacgacgttataa

SEQ ID NO: 15 - DNA, 15983 bp, Helicoverpa zea
atgagattagaaacgaggcacgctagtgccgccaccaagttccgcctgctcatgtggaagaactttctgcagcaatggagacatcggctacaaactgtcgtagagctactattgccagtcgtgacaatggcgctggtgctcatccttcggtggcagataccgccttatcaaatcgatacactcacttaccccgcgttgccagcgcacacactcaactactctaccaatatcctgtaagttaccacaaaatattcttcttttaaattgctaatatccaacgtttttcatctttggttggatgacaaccaatttcatcaactcgtatgcttatttatcttaatccatcctaaaaaatctgtcaagcttcttgcccggtaatcaatatttaaggcaatcagattcagttgtgtgcctttaaatattacagatattgtaaagatagcgcatttatagttatagctaggtcctactaacttgctccactataaacattttataacataaacaaacatttattcacgaacattttattcacagacagcattcgttttatatattttctaaaacagtttctagagaggtatattcagtacaggatgtgtgtttatcaaataatctttgaagttcatacatatcctttatcatatctaaaagctaattcttttgctaaacatactgtaaaattataagtggtaatctgtatgtttagtcaattttggtgactaagcaatagataaaacagagatgttttattgattaggtacagttaaagattgccgaagttacaatcgcggtaatttatgaaagcattaaataaacataaggattcgatgatttcctcagaacattaaatgcaaatataaaggtagaattccgtgggtcgcggcttacgcagccgcgcgcggcttacgacagtcgtcggccgcgcgcggctgtcgtagccgctatccacggaattctacctttaggtacctacttgtcgccttcaatcattgcagattttcatttctagagactatagctgctgaaagttttctttaaatattgctacaataaatacatttcaaatatgagaatttccgtttattattcaaagttatgaaatttaaataatatacaatgagcatctgtattattaaaactcatagttactcttatgtctatgcactctaggtatatgttttcattcgtgaacacatttgctatttgtactcactccataagccgagataaggcgataatattacatctcgtggttataatcgtccctaatgtttaatgaaattcatttttcagttttgccatgaatatggaagaattatcaattgcatactccccggcaagtccagtgttagatgatgtaatgagaactgctgttattaatttactaacagccaatatgaaagatctgattcctatttttattgataacttaccaccggggatagccaatataacatttccaccagatatgaacttaaatacgtcggccattgaggagttcgtgaagtcacgaatacgagtcgtaccttataacagcagttatgaaataagagggatctacgttgacgaagaaactacacgcagcattatcgctgccgtcgagtttgacgataaactatatggtatgaataaactcgattgcttacacaattttttgtgcctatttccagctactgatattcaggtaatgttacaaacagcggcttctaatctatttcaggagcagaacagttgtcaaataatttatcttattcgctacgttttcctgagagaccccgtctcaattcgtttttccaaactggagggcgcacctggagatctgacggagtattcccggttttcgaagtgcctgggcctagatttcctcactcgtgggaaggtggtaatgacccaggtaaatatatttattcgctttaggacgtctggcaaacactaattaaaaaatatattaactttgtttaaaataaaaacaagtaaggtatcgaaactaacgaaaataaggtattgttaggattaggtacatactgaaattaatcaagtattgtgttaactacaataaatattgtgcacaggttacgttaacgaaatgttcgtggcacttcaacaagttatttccatggagctggtatcaagggcaaccgggttggacttgaagtcatttagggtgaacatacagaggtacccgcacccgccgtaccttcacgaccagtcagtggatctgctgcaatttatgttccccctgttcatcatgttgagcttcagctacactgctgtcaacattgcacgggcggtcacagttgagaaggaattgcaattaaaggtacctagatagtaattccaaggtacaatgttggaggataccaattaaataaccttgctaatttttattatgttgttttaggaaactatgaaaattatgggcctccccacatggttgcactggacagcatggtttgttaaacagtttatctacctatcaatcacagctgttctgctagttgtgttgctaaaggtgagattttcaaaattagtacacagattcttacgtagtttagatttaataataacttatgtattgtactgtacttattgtttttaggtaaattggtttactaacgacgatggcttcagcgaatatgctgtatttactaatacaccttggacggttttgctattcttcttgatactgtatttatcttgcgcgatatttttttctttcatggtaagcagtatattttcaaaaggtatgctttcttcttatttctttttaatttaacaacctacattaacatatttaacaatatattatttttgtggtaaaggtagtacggccgcgttgtttatggcggtggcatggttcctcacttacatccctgctttcctcctggccatggatatcaatatgtcgactgcggtgcaggtcatcacatgcttcagtattaactctgcgatgtcctatggtttccaactaatgctcgctaaggaaagcactggaggtaaattattacgagagtttattatacgagccacttaaaataattacaatataaatgctctgctccaaagtttgtttgatccgaatcagggaatagaaaaattatgcccaataattataattttcctaatttgttgcaccgcttcacacgtaacacgattcgaacaagacggagtgcgagtggcggtaattgaagtcgatgtgccacattagcgtaggagaataaattctaacatagcgactggtctaagtgctaaattgctttactgaacgtattcgctcaattagggctgcagtggggcgacttcatgacgtcaccagggacggacaccacgcgcttcgtgttcggccacgtggtcatcatgctggtagtggactgcctcatctacatgctgatcaccctctaccttgaacaagtgctaccaggccccttcgggactcccaaaccttggtacttccccttccagttgcagttctggttcccaaattataaatcgaaaggtaagatattttgtagagttgtactctacctaatttatgaatcaaattagtaatttaaaatagtcattgttacaagcattaatgtcatagcaataatcattatgctacaaagtcgaattaggtatctttgtaataaaatccaatgactcagaaatgctatgattttttttagatgctggattaattttcgaaaatgataatagtgaattcgatgatattataaaagaaaaggatcccacagaccacgaagttggtgttaaaatgcaagtaagtttatcatcacttaaaagtaacattaagtacttacacactaatatattatgtgaaaatatatttgttaattcattaaaaaaaatatttgtacgtatttatttacagaatttaacaaaaatctttgggaataacatagctgttaacaatttatctttgaatatctatgacgaccaaatcacagttttacttggtcataatggtgctggcaaatcaactacaatatctatgttaacaggtttgtaataaaatattttttatttatgctcttctttaaccatgtttttaaatttaattaacaaggattaagtgcttgtatttagtttgtacccgggagcttcgttgattttcaatgacattattagatggagattagggataagagtactttttgatgccttgtcactatctatgtctatatcctccgtatgcaaagttgcgataaaatgcaattcattcaagttttacttaaaatactaggatgagtgaagatgtaacatacgttgcagagtgggtgcaagtcaattgattcactactatgaagtcttgtttctggtaataagcaaagaccggaactattgcgcaacattgttattgtcaagtgggcactagctttaatgtaacgatgccggtcagtgtgttaatctatctaaatagagttgaaggttcttcaaacatttttctgtctaacgacgaacacacattatatgtaatttcagcggcacgcggaaaccattttataaaagagtatgaattgtcccagttattatggtccgtttttaccacccatttaaaaaaaaaagcagttgacgactcgtattgtccgaggttttacatgggcggaaagatgtaaataaaatcgaaatcatatacatatgtcataacatactttaatttttgaatattttgtgtttcaggaaatttaaaggtaactcgcgggacagtgaacgttgccggatatgatatgacttctcaaagctccgcagcccgttcccacattggattgtgtcctcagcacaatatactgtttaacgaactcacggtcaaagaacatttggaattctttgctagactaaaaggatttaaaggcaaagaactgtatgaagagatagactcacttattgaaaaattggaactacaggagaaggtatctacaatttatgattatttggaatttgtcagttcagttctgtctctatcaattagagagatttttttaattagagcggcctcatatcatatttcttcaagttacaaatggaactagaaagtgattgggcgtacctgcaatacaagggcggatccagcttcggcgccaggggggggtcacacagtcgtggtcaagtcacaaaaaatattatattgtagcgtatacttcttttaatattaaaagtagtagtataaatacaataagcgcgatcgtagcgagctcgaaatttttacgaatgtttaagaaaagtgttttcatgtagaaaatggcccgagcagagtgagcgcaatttctcgtatttatactacacacacgcgccaaacaaaaaagcgcgagcgaagcgagcccgaaaatttttgttctggtcgaggactaaggttaaaaaagtgttgttatgtagaaaatggtccgagctgagcgaacgcgatttcttggacataatcacagacgcgaaactgaaaaaagcgcgagcgaagcgagcgcgaaaattttttttcagttcgaggactaaaagtagatgaaaacgctttcttgatgtataacaaatatacagcacaaatacaagaaagcgcgatgttcatgagatcagggccgtctagcttatgtagcgcctgggtgcaatcatcacccaagcgccacacatagtacatattgatatcgaagtactttacaaggaatataaataagaacgttcgaacgaaaatcacttttttggactaagaaaaaatgtttccaaatcatcgtaggctcaattttaggctcactgttggccagatttaagttatgaaagtcgagacgggacaacataattgtaaaaattagcgcgatcgaagcgagcgcgcaaattttttaattttgggacacaaaaagttcataagttttaaaaagcgctgtaaagtaacaagcagtcggaagcgcaaactgttttgtttttgaggaccccataaatattttttttttgctatatttgacttatgaagttatcaagggaaggcatatataatattgcgcgagcgaagcgagcgcgaaattttttgaccctacgacacaaaagcacctgattaagtacattgtaaagcaacaagtagccgcaagcgcagcgaacgcgaactttttaaaattatttgtttgaagactcacaaatcagactgggaattacgtacaattaaaaaggaaccgtgattagagcgagtgccatttgtgttcgttgattcggtgcgtcaataaaaataataaacaatcagaaatacagtacctacagacatagtcatttactcgcgagcgtagtgagctagcattttttcacttagttggaggatgttaaaagttaaaaaaaaataatcaaaatgatcaatccaacaaagcgaaggcgacttttttgtcagtatcatgatacaatgtggaacttccttatccaacgggtgtaatttcttcgaaatttcctggtggtggggcgtctcgctgcgcccctgatatcgaggcgcccgggttattcgcacaccctgcacttaggttaagacggccctgcatgagactaatacattacagcatgtaatgtagacaacccggacagcggattcttagtaacagggtcccgcttcctccttttgggtacggataaagagtaaataaagaatagagagcgagcgttgcgagcacgaattcttcagcaaaactactctacctgtaactatgtaggtatctacctattcactcgaaataaaaattatcttatacttataacaaaaacataaaacatcgtgtttaaccatttcaattattggtcctcttaggtcctgaaccaggcccaggggggggtcatgaccttgtgacctaccccctggatccgccgttgctgcaatataagacatgttatttgtttcacagcgtgactacccctcaaaaggtctgtcaggaggtcagaagcgtcgtctttgtgttggtattgctctgagtggggcggcacgagtggtcttactcgacgaacctacgtctggcatggatccttcatctcgtcgagcactgtgggaactcttacagaaggaaaagaaaggtaggtcgaaattcaatcgttagaacagagaattaggtatttctttgtataaagtatttttgttttaaattattattaatataaagggaccgacatataaacctaatagtgatggctatagtcgcatgttaatatgtttattatatcgttaggtcgctcgatgatcctgacgactcattttatggacgaagcagatattctcggcgacagagtggctataatggcaaacggtagactgcaatgcgtgggctctccgtatttcctcaaacgtcattatggcgtcgggtataccctggtgatcgttaaggacacagacttcgactttgtgaaatgctccgtacttatcaatagctatattcctggtactattgttaaagaagatcgaggtaagtttttactggttaaaatgttgtctgatatatgatccatcaaactataattgcgtccttttattgagaatactagcttttgcccgcggcttcgctcccgtcaactgacttctctactttaccctattttttttttcataagaaccttctcctgacaataacaaacacaacaaaaaaagaattagccaaattggtccaggttgagttatgcgcttaccaacacattttgcgattcatttttatattatagattaaatataaacaaatatttcttttttaaggaacggaaatcacttataatttggtaaacgattactcacacgtttttgaagaaatgttgaatgatttggaaagaaatattgataacatcaaatttaaaaactatggtttagttgctactacattagaagatgtctttatgtcgtaagtattttctataaatgtttagttgttagtacgttagcctaattttggtaagtggggtgataacaaagtacttatgttgcagcgttggtgcagacttaagtccaattaattccgaatctgacgatgctattactactactactgactcgactatcgatgatatattaaaacaagaaatcgattcatctttggaagaacgtaagttgtccaaagatattgggtattttaaacgaaaaaaaaaatcattagatatcctttttacaacttgattacaaaaacggtccttgttttctagtggataaggacgagagtaacgtgacgggcctccgcttgttcggtcagcaagtgctggctgtatggatgaagcagtggctggtgctgatccgctcgccatgggtcatggtactgcagtttttggcgccagtggtactcatcaactccacgctaggagttctgcgttacgtcatgtctttatcaccgaccattagaactaggtggttgtcgttggaagaagggtgagttaaatagtaaaatcactttattgacatcggaaggcgcaacgcctattgaccggttaagttcgtgaaattcttttatctcttatatcatcaagtaaatctaggtacttatattgggaatcgatcgtatcgacgcctagtatctgcaaggacataaaatacttacctacatattgcttcacatcaacaatctacctaaatgcaataggactcttgtgtcagagactgcgaatcttatggaacttacagtacctaaatacataacatacatacatacttataacatcataaattattttcaggtatacggaaagcgaaactctgctcagcttcaacggtagtgtagcgtcatcagtgggtgccctagccgcgcaagcataccaaagcctgttcgccaattctggtgttatggacatggaaatcaacgctatcggaagccagccaatagaagaatattatctaaatagagtgagtcactatacaatttcgaagtcggcaaccgatatatcatgatttcgataaagacaaacagggatcaaaacttcgataaataaataatcacctgctatatcataattacaagtcattatgagtgagattcatacattattgtcatgatggatggatggatttacatttttacaaatttaaatgaactcatccataaaaaaaaatactcaatccttaggctataaatactataaatactcaatcagattccactgaaacggagagctgtggagtttttaaacatctttcgttaggtattttagttatactgaaatatcctttctccatccactcagtaaatctattggttatataaaaacctctaacttgtaggtacttgctaataagagattttttgcttacacagacaagtgatcccgttgtgatgggttcgctgcggcaccgcttgctgataggctccacatttgacgacaactctgctaccgcctggttcagtaactttggctaccacgatgttgctacatcgcttgcggcaatccactcagctattctcagatctaaaaactctgatgcagtactcaatgtatataatcatccgttggaagcttcgtatatagatcaggtaagttttacagacgaactgtctatatctttttactctttctttttaacctatacagcttagtcgtagcaattagaattaatcgctaatctcttcaatgaatgttttcagagtgacgtgcagactatgatagctatgttgtccatgcagttgtcctctggcatcggcagtagtgtgagcattgttagtgcggttttcatcatgttttatatcaaggtaagtacattatttaacctcgaaaagtattttctgctcttttttttttttttttttttttttataatttcgggacacctttttcacacacggtcggttaaccccatggtaagttatttattaacttgtgttatgggtgctaacacaactgataaactacatatagctacatatatacatatttataaatacatatcataacacccagacaacggccaacaagcatgctcatcacacaaatgtcgaccgaaccgggaatcgaacccgggacctcaggttcggcggtccggcatgatgaccattgcgccatcgaggtcgtcttatgtaccgattctttcaaacttttgtgacttcttcaggttataacggttcgtttttgttttcgcaggaacgtatgtcgggggcaaaacttctacaaaatgcagcaggcgtggcgccttctgtgctgtggggcggcgcagcgatcttcaattggttttggttcctcatcacttgtgtttccatcgtcatctcgtgcgtcgcttttgatgtcatcgggttatcgaacgtgcatgaattaggtaatatggtgtttcctagcagtgccggttttaactctaccagcgtcctgggcgagatttctacggcgccctccaactgcaactcaagcccatacgaaaaactgagacatatgtagttatcaatttgcgcgagcgaagcgagcgcgaattctgtttttatagttaacaagtcaaagcaaaaattaagtaaaatgtagcccaatcgtacctacataagttattgctggttggtgaatgcaaaaacatgggtacatggcttctgattgcgcgagcgaagcaagcgcgaaattatttgttatattttagaactcaaaacaaaaattacttgactctttgttggtgttggcgcctgtgtataaaaattttgggacttaaagtagtaccgtaaataagaaagtccatggaaactagaagttactttcttattaataaaagaacttaaaaacgacgctacctttttgctagggtagactaaaaaattgttgaaaaataaaaacacctgtaaagtgaagcaagcccgaaaatttttgagttttggatcactaaaagtcctaaacatatcatcatcctccgagcctttttcccaatcatgttggggtcggcttccagtctaaccggattcagctgagtaccagtgctttacaagaagcgactgcctatctgacctcctcaacccagttacccgggcaacccgataccccttggttagactggtgtcagacttactggcttctgactacccgtaacgactgccaaggatgttctatgacagccgggacctacagtcctaaaagtcctaaacatatacaggaattaattttaagcagtgcgcaaaaaaaaactggtggtccggaatcattaacagaacatatctgcatcttgagtaaaacaaaatttttcattcttttgtccgccctaaaatcagcaaaatatggataccaactattcttacgcgcttggagcagcgctcgcgtcagttaaccggtttcgctttgcagcgcgcctactataacgactgtgaccgtacaatcggttacaaaataaacatctttcgatatcaataacttttcttttttgtactaaaacacgacaaaacaaaaatatacgtatctataaaacttacttaattataggttgacaaagtttgcgcactggataaaattcattcctgtataataaaaggcgactgtgaagtgaagaagccgcgagcgaagggagcgcgaattttttgagtattgggacattaaaagtagctgtatgtcataaaaaaaacaaagtgtaaagtaaagaaatcgcgagcgaagcgagcgcgaaaatttttgagttttgggacatcaaagtagtgtttgttcgagaatttctcggaattaaacacaaattcgcttcggcgcccctgagcccgcagcgcccgggttattcgcacaccctgcaccataagttaagacggccctgatgctatccatacatttggatacagttcttgtaagttgcgatctttgatgaaatttaaaacaaaaagaagagtaacattctgatcaaggattggttggggccgcctgtggcgcccccttatgaccggcgccctgggccgtcgcccaaccgcgccctaccctaaagccgctactgtttcctagtagcagtccttttaaataatgtttaaaagaaactgattaaatgtgacttgtacctttataggctggtactaattaataattgcaattattatttcaggtcgaatgtttttgtgcgtcatggtatacggtgcggcgatgttgccattagtgtaccttttgtcgcttaagttcaagggaccagctgtcggcttcgtgggcttctatttcctcaacgtgcttttcggtaagttataggatagccaaaagggttcaagtatatatataagtattgagtattgtatactaaaattaagtttcagagtcacccataatgaatctctgctttctttttccctttttgtaatgaccgtattattcctgaatcgggtaggttcctgaaaattcacttgtgttgtattctacaggtatgatgggtgcgcaggtggtggaggcactatcctctcctatgctggacacagagcaagccgcccacatccttgactacttactgcagttctacccgctttacagtcttgtcacttctatcaggtaggtactcgctgcaacaagcccaatatcctacgttctaaaccatataagttaaagtaatcacacattatattttcatttgggtggtgatctcaatccgtataatttcgatttgcaggtttttaaatcaagtcggcctacgggagtatacttgcttacaaggctgtgaatacttgcaggcagtatacccgaatctagagtgtagcatggcaagcatgtgcgaattccacagtaactgctgcggtaagtggcatagcttcccataaatatgaattaattgagatgataataggtatcatcaatttatttgtcgatttctccttgcagttcgtgaaaacccatacttcgattgggaggaaccaggcgtcctgaggtacttgctcagtatgtgcttctcctgcctaatcttctggttgctgcttatgaccattgaatacagagtggtgcaaaaggtaattatagccctgtatttacatcattaaaaattattagtccattgaggtattttctaaaattaaaactgtaactattttttgacacatttctatacacttttggtcaaagtttaaaagatgtttctgacgtttggtttcgatgtgtcgtaggtgttcacattcaagaagactcctcctccaatagacgagagcacgttagacgaggacgtgatgacagaggcgaggcgcgcgcgccaggtgccgccgacacgccgcagcgaccacgcgcttctcgctcacgacctctccaagtactacgggaaacatctcgccgtagaccaagtctcgtttagtgagtcaacttcgcatttaaaaaaataatttgtattgtaggtacatttatatacattgtccggaatttccaaattaacctttccattattcatcaaggtgtgaacgacggcgaatgcttcggtctattgggtgtgaatggtgccggaaaaacgaccaccttcaagatgctgatgggtgatgagtccatttcaagcggcgaggcgtatgtctccgggcactcggtgcagaggaatctcgatagagtacacgagaatattggtcagtagccaaaaaacctcttgaaattttaatataaaaacttttattactgctgacacgtaggttcgtgtaaccacgtacatatattcatgtctaatttatattgcaggatattgtccgcaatttgacgcattatttggtgagctgacgggtcgccagacactacacatgtttgcgttgatgcgcggcttgcgtttacgcactgcagcaccttcggctgaaacactcgcacatgcgcttggcttcttcaaacatcttgataaaagggtaatcttttatttctttaaatatatgtctaggcctactgtattgtagttgtcataacatttattttgaatttaatttttcaggtgcatcagtattcaggcggcacgaaacgcaagcttaacacggcgatagcattcatgggacgaacacggcttgtgtttgttgatgagcctaccactggagttgatcccgccgctaaacgccacgtgagtacttcgacctaatcagcgaaaaccagaacaagtagactcaagccatctttcgataggatatagtgcatgccctcgagtggtcttaaggatactagattcaggtttttttttggtccttaacattcagcaatatagaatccaacgtttttaaattgatcaggtatggcgcgctacccgcggcgtgcagcgagcaggtcgcggcgtggtgctgacgtcacatagtatggaggagtgcgaggcgttgtgttcgcgactgaccatcatggtcaacggtcgcttccagtgtctgggaacgccacaacatctcaagaacaaattttctcaaggtaaaggaaaaaatcgatagtgtcgttcatcggattcattcgtgttttgcataaccgaatgtagagcaggcccctaccaatgttttacataggtctgtctaataagaaaacccctttgtaagtttaatggtataccagtcgttgatttacaaatgacaccctgtgacaaagtctttttgcgttttcccagtcacgcagagacctactgtcattaaaagatggtcacccattctcccattcacaccaggacagagcgcgttaacggcttattgataaataactttaatttgtttcaggctttactttaatcattaaaatgaaaactgacgacagtgacagcgacacgcagtcagtaaacagcactaccagcgtagtagatagtgtcaaactatacgtctctgggaactttgaaagtccaaagataatgtaagtaacaaaatctatttcaagttctggagctatataggataagttctaattctatattagaacttaaaaactgaatttatcactatcgtttcaataggcatgatgacagtaatcaaaaatcattaaaataatatatttattaaattaaacttgaagcttggtatataaaacgcgcattgttttctttattaataatgtgattaatatgtatttttataaaataaaattacgaaatagggcaatccgccatgatatcttgaacaccaatcagagctcgtgacgtcatttctcaaaacaactcgcgcgaaagcgcgcgaacgtcacatttcgttattgtgaacttccactgcgatctttgtttttaattaattagttgtttataacacgagttatggagcccggatttatcaatgcaaacagcgctagcattgatatgcagcctagaattgatatcataatgctgggaaagtttttggcatcaaataaagatttttgttcagctgaatttagaaatcttaaaacttccatgtaagtatactagtttaattaaaaaacaatgagagatgaaacctaacctcgaaatagttatttacattataaactatgctagaatctagagaactacgtaataacttacatcaaagtgatcttcacaaaaataaagttgtaccctgggcaatattgcttttgaatctcgccttgcaagtttaagccgcttgtttcgtatttttgtaggtattgtgaggaacgtacacaaataactgaccccacaacacctatgccctttcgaattcatatttaataacacaaaaaacttaattatcgcacgagcgttgttcacttgcgtcttgtaatttcagtcgtgacgtcacaagtgacaccatgacgctgacaagcgttttcgcgccggattcaaagtggacagagaaaaatgcaattatttgataaaaaatcttcgcattttcttaaaattaataatttttcatataaaatagacgtatacctacattatacaaaggaatttaaaaatttgtcatcatgcctattagaattccagcagtggtgtgctttattggggtaattggctgctctttatatagataattctatttttcagggaagagtatcatggtcttctaacttactacttgcctgaccgtagcatggcatggtcacgaatgtttggtatcatggagcgcgccaaacagatcttacaaattgaggactacagcatatcgcagactaccctcgaacaaatattcttgcagttcaccaaataccaaagagaagaaggaacgacgttataa

SEQ ID NO: 16 - DNA, 5229 bp, Helicoverpa zea
atgagattagaaacgaggcacgctagtgccgccaccaagttccgcctgctcatgtggaagaactttctgcagcaatggagacatcggctacaaactgtcgtagagctactattgccagtcgtgacaatggcgctggtgctcatccttcggtggcagataccgccttatcaaatcgatacactcacttaccccgcgttgccagcgcacacactcaactactctaccaatatcctttttgccatgaatatggaagaattatcaattgcatactccccggcaagtccagtgttagatgatgtaatgagaactgctgttattaatttactgacagccaatatgaaagatctgattcctatttttattgataacttaccaccggggatagccaatataacatttccaccagatatgaacttaaatacgtcggccattgaggagttcgtgaagtcacgaatacgagtcgtaccttataacagcagttatgagataagagggatcaacgttgacgaagaaactacacgcagcattatcgctgccgtcgagtttgacgataaactatatggagcagaacagttgtcaaataatttatcttattcgctacgttttcctgagagaccccgtctcaattcgtttttccaaactggagggcgcacctggagatctgacggagtattcccggttttcgaagtgcctgggcctagatttcctcactcgtgggaaggtggtaatgacccaggttacgttaacgaaatgttcgtggcacttcaacaagttatttccatggagctggtatcaagggcaaccgggttggacttgaagtcatttagggtgaacatacagaggtacccgcacccgccgtaccttcacgaccagtcagtggatctgctgcaatttatgttccccctgctcatcatgttgagcttcagctacactgctgtcaacattgcacgggcggtcacagttgagaaggaattgcaattaaaggaaactatgaaaattatgggcctccccacatggttgcactggacagcatggtttgttaaacagtttatctacctatcaatcacagctgttctgctagttgtgttgctaaaggtaaattggtttactaacgacgatggcttcagcgaatatgctgtatttactaatacaccttggacggttttgctattcttcttgatactgtatttatcttgcgcgatatttttttctttcatggtaagcagtatattttcaaaaggtagtacggccgcgttgtttatggcggtggcatggttcctcacttacatccctgctttcctcctggccatggatatcaatatgtcgactgcggtgcaggtcatcacatgcttcagtattaactctgcgatgtcctatggtttccaactaatgctcgctaaggaaagcactggagggctgcagtggggcgacttcatgacgtcaccagggacggacaccacgcgcttcgtgttcggccacgtggtcatcatgctggtagtggactgcctcatctacatgctgatcaccctctaccttgaacaagtgctaccaggccccttcgggactcccaaaccttggtacttccccttccagttgcagttctggttcccaaattataaatcgaaagatgctggattaattttcgaaaatgataatagtgaattcgatgatattataaaagaaaaggatcccacagaccacgaagttggtgttaaaatgcaaaatttaacaaaaatctttgggaataacatagctgttaacaatttatctttgaatatctatgacgaccaaatcacagttttacttggtcataatggtgctggcaaatcaactacaatatctatgttaacaggaaatttaaaggtaactcgcgggacagtgaacgttgccggatatgatatgacttctcaaagctccgcagcccgttcccacattggattgtgtcctcagcacaatatactgtttaacgaactcacggtcaaagaacatttggaattctttgctagactaaaaggatttaaaggcaaagaactgtatgaagagatagactcacttattgaaaaattggaactacaggaggagcgtgactacccctcaaaaggtctgtcaggaggtcagaagcgtcgtctttgtgttggtattgctctgagtggggcggcacgagtggtcttactcgacgaacctacgtctggcatggatccttcatctcgtcgagcactgtgggaactcttacagaaggaaaagaaaggtcgctcgatgatcctgacgactcattttatggacgaagcagatattctcggcgacagagtggctataatggcaaacggtagactgcaatgcgtgggctctccgtatttcctcaaacgtcattatggcgtcgggtataccctggtgatcgttaaggacacagacttcgactttgtgaaatgctccgtacttatcaatagctatattcctggtactattgttaaagaagatcgaggaacggaaatcacttataatttggtaaatgattactcacacgtttttgaagaaatgttgaatgatttggaaagaaatattgataacatcaaatttaaaaactatggtttagttgctactacattagaagatgtctttatgtccgttggtgcagacttaagtccaattaattccgaatctgacgatgctattactactactactgactcgactatcgatgatatattaaaacaagaaatcgattcatctttggaagaactggataaggacgagagtaacgtgacgggtctccgcttgttcggtcagcaagtgctggctgtatggatgaagcagtggctggtgctgatccgctcgccatgggtcatggtactgcagtttttggcgccagtggtactcatcaactccacgctaggagttctgcgttacgtcatgtctttatcaccgaccattagaactaggtggttgtcgttggaagaagggtatatggaaagcgaaactctgctcagcttcaacggcagtatagcgtcatcagtgggtgccctagccgcgcaagcataccaaagcctgttcgccaattctggtgttatggacatggaaatcaacgctatcggaagccagccaatagaagaatattatctaaatagaacaagtgatcccgttgtgatgggttcgctgcggcaccgcttgctgataggctccacatttgacgacaactctgctaccgcctggttcagtaactttggctaccacgatgttgctacatcgcttgcggcaatccactcagctattctcagatctaaaaactctgatgcagtactcaatgtatataatcatccgttggaagcttcgtatatagatcagagtgacgtgcagactatgatagctatgttgtccatgcagttgtcctctggcatcggcagtagtgtgagcattgttagtgcggttttcatcatgttttatatcaaggaacgtatgtcgggggcaaaacttctacaaaatgcagcaggcgtggcgccttctgtgctgtggggcggcgcagcgatcttcaattggttttggttcctcatcacttgtgtttccatcgtcatctcgtgcgtcgcttttgatgtcatcgggttatcgaacgtgcatgaattaggtcgaatgtttttgtgcgtcatggtatacggtgcggcgatgttgccattagtgtaccttttgtcgcttaagttcaagggaccagctgtcggcttcgtgggcttctatttcctcaacgtgcttttcggtatgatgggtgcgcaggtggtggaggcactatcctctcctatgctggacacagagcgagccgcccacatccttgactacttactgcagttctacccgctttacagtcttgtcacttctatcaggtttttaaatcaggtcggcctacgggagtatacttgcttacaaggctgtgaatacttgcaggcagtatacccgaatctagagtgtagcatggcaagcatgtgcgaattccacagtaactgctgcgttcgtgaaaacccatacttcgattgggaggaaccaggcgtcctgaggtacttgctcagtatgtgcttctcctgcctaatcttctggttgctgcttatgaccattgaatacagagtggtgcaaaaggtgttcacattcaagaagactcctcctccaatagacgagagcacgttagacgaggacgtgatgacagaggcgaggcgcgcgcgccaggtgccgccgacacgccgcagcgaccacgcgcttctcgctcacgacctctccaagtactacgggaaacatctcgccgtagaccaagtctcgttcagtgtgaacgacggcgaatgcttcggtctattgggtgtgaatggtgccggaaaaacgaccaccttcaagatgctgatgggtgatgagtccatttcaagcggcgaggcgtatgtctccgggcactcggtgcagaggaatctcgatagagtacacgagaatattggatattgtccgcaatttgacgcattatttggtgagctgacgggtcgccagacactacacatgtttgcgttgatgcgcggcttgcgtttacgcactgcagcacgttcggctgaaacactcgcacatgcgcttggcttcttcaaacatcttgataaaagggtgcatcagtattcaggcggcacgaaacgcaagcttaacacggcgatagcattcatgggacgaacacggcttgtgtttgttgatgagcctaccactggagttgatcccgccgctaaacgccacgtatggcgcgctacccgcggcgtgcagcgagcaggtcgcggcgtggtgctgacgtcacatagtatggaggagtgcgaggcgttgtgttcgcgactgaccatcatggtcaacggtcgcttccagtgtctgggaacgccacaacatctcaagaacaaattttctcaaggctttactttaatcattaaaatgaaaactgacgacagtgaaagcgacacgcagtcagtaaacagcactaccagcgtagtagatagtgtcaaactatacgtctctgggaactttgaaagtccaaagataatggaagagtatcatggtcttctaacttactacttgcctgaccgtagcatggcatggtcacgaatgtttggtatcatggagcgcgccaaacagatcttacaaattgaggactacagcatatcgcagactaccctcgaacaaatattcttgcagttcaccaaataccaaagagaagaaggaacgacgttataa

SEQ ID NO: 17 - protein, 1742 aa, Helicoverpa zea
MRLETRHASAATKFRLLMWKNFLQQWRHRLQTVVELLLPVVTMALVLILRWQIPPYQIDTLTYPALPAHTLNYSTNILFAMNMEELSIAYSPASPVLDDVMRTAVINLLTANMKDLIPIFIDNLPPGIANITFPPDMNLNTSAIEEFVKSRIRVVPYNSSYEIRGINVDEETTRSIIAAVEFDDKLYGAEQLSNNLSYSLRFPERPRLNSFFQTGGRTWRSDGVFPVFEVPGPRFPHSWEGGNDPGYVNEMFVALQQVISMELVSRATGLDLKSFRVNIQRYPHPPYLHDQSVDLLQFMFPLLIMLSFSYTAVNIARAVTVEKELQLKETMKIMGLPTWLHWTAWFVKQFIYLSITAVLLVVLLKVNWFTNDDGFSEYAVFTNTPWTVLLFFLILYLSCAIFFSFMVSSIFSKGSTAALFMAVAWFLTYIPAFLLAMDINMSTAVQVITCFSINSAMSYGFQLMLAKESTGGLQWGDFMTSPGTDTTRFVFGHVVIMLVVDCLIYMLITLYLEQVLPGPFGTPKPWYFPFQLQFWFPNYKSKDAGLIFENDNSEFDDIIKEKDPTDHEVGVKMQNLTKIFGNNIAVNNLSLNIYDDQITVLLGHNGAGKSTTISMLTGNLKVTRGTVNVAGYDMTSQSSAARSHIGLCPQHNILFNELTVKEHLEFFARLKGFKGKELYEEIDSLIEKLELQEERDYPSKGLSGGQKRRLCVGIALSGAARVVLLDEPTSGMDPSSRRALWELLQKEKKGRSMILTTHFMDEADILGDRVAIMANGRLQCVGSPYFLKRHYGVGYTLVIVKDTDFDFVKCSVLINSYIPGTIVKEDRGTEITYNLVNDYSHVFEEMLNDLERNIDNIKFKNYGLVATTLEDVFMSVGADLSPINSESDDAITTTTDSTIDDILKQEIDSSLEELDKDESNVTGLRLFGQQVLAVWMKQWLVLIRSPWVMVLQFLAPVVLINSTLGVLRYVMSLSPTIRTRWLSLEEGYMESETLLSFNGSIASSVGALAAQAYQSLFANSGVMDMEINAIGSQPIEEYYLNRTSDPVVMGSLRHRLLIGSTFDDNSATAWFSNFGYHDVATSLAAIHSAILRSKNSDAVLNVYNHPLEASYIDQSDVQTMIAMLSMQLSSGIGSSVSIVSAVFIMFYIKERMSGAKLLQNAAGVAPSVLWGGAAIFNWFWFLITCVSIVISCVAFDVIGLSNVHELGRMFLCVMVYGAAMLPLVYLLSLKFKGPAVGFVGFYFLNVLFGMMGAQVVEALSSPMLDTERAAHILDYLLQFYPLYSLVTSIRFLNQVGLREYTCLQGCEYLQAVYPNLECSMASMCEFHSNCCVRENPYFDWEEPGVLRYLLSMCFSCLIFWLLLMTIEYRVVQKVFTFKKTPPPIDESTLDEDVMTEARRARQVPPTRRSDHALLAHDLSKYYGKHLAVDQVSFSVNDGECFGLLGVNGAGKTTTFKMLMGDESISSGEAYVSGHSVQRNLDRVHENIGYCPQFDALFGELTGRQTLHMFALMRGLRLRTAARSAETLAHALGFFKHLDKRVHQYSGGTKRKLNTAIAFMGRTRLVFVDEPTTGVDPAAKRHVWRATRGVQRAGRGVVLTSHSMEECEALCSRLTIMVNGRFQCLGTPQHLKNKFSQGFTLIIKMKTDDSESDTQSVNSTTSVVDSVKLYVSGNFESPKIMEEYHGLLTYYLPDRSMAWSRMFGIMERAKQILQIEDYSISQTTLEQIFLQFTKYQREEGTTL

SEQ ID NO: 18 - DNA, 5232 bp, Chrysodeixis includens
atgaagatgaggcgagaggctaagcccgcgggcgcgttcatgaagttccgcctgctgatgtggaagaacttcctgcagcagtggcggcaccggctgcagacggtgacggagctgctgctgcccgtgctcaccatgacgctggtgctggtgctgcgctggcagatggagcccagcatggtcggcaccctcacgtacccgccgataccagcacacacactcaactattctacagccattttagcgggcatgaacttaacacagatgtccatagcgtactccccaagaagtccagtattagatgacgtggtcagaactggaattacaaacttattagttgcgaatgcaaaagatctgcttccaatttttgaaaatatttcaatacctggtttaccagaaatagaacttccatctattcctgaagatttcaactctacactaattgtagagttcttgaagtctcgaataaaaatcgaggcttacaacaatagtcatgatctaagaggactgtacatccgcgaggagtccactcgcgtggttatcgctggcattgagtttgatgacaaactatatgatgcggaaagcttgtcaaacaatttatcatttgcactacggtttcctgaaaggcctcgaatgaactcgtttttccaacgaggggggcgtacttggcgaaccgatatagtgttcccattatttgaaatgccaggaccaagatatccatggtcatgggaaggcggcagagatccaggctacgtcaacgaaatgtttatcgcgctgcaaaacgctatctctaatgagttgatatccagggctaccggggaggacttgaaaaagttcagggttaacgtccagagattccctcacccgccttacatactcgacatggcggtggatttactgcagttcatgttcccgatgttcatcatgctcagcttcagttacactgctgttaacatagccagagcagtcaccgtagaaaaagaattgcagctgaaggaaacgatgaggatcatgggtctacccacatggctacactggacagcgtggttttgcaagcagtttctctatcttctaattacagcgattctaattatagtccttttaaagatacattggtttactaacgaagagggcttttctgaatacgcagtgtttactaataccccgtggacggtgctgttctttttcatggttttgtacttatcatgcgctatatttttctgttttatgataagcagtttcttttcaaaaggtagtacagcggctttgtgcatgggagtggtctggttcctatcttacgtccccgctgtcctcttggctatggacatcgatatgtcgacagcaatgcaagtcttcacgtgccttagcataaattcagcgatgtcttacgggttccaacttctactggccaaagaagccgttggaggtttgcagtggggcgacttcatgtcgtcaccggcggcggagaccaaccgcttcgtgttcggccacgtggtcatcatgctggtggtcgactgtgtgctgtacatgctcgtcactctgtacctggagcaggtcatgccagggccctttggcacgcccaaaccctggtatttccccttccaattgaaattctggttccctaattatagttcagatgtcggttttattttggaaaacgaagttagtgagtctgaagatataattaaagagaaagacccaattgaccatacaatcggtgtcaaaatgcatgatctaacaaaaatttacggaaataatgtagcggtcaatcatttatctttgaatatttacaacgatcagattaccgtgctcttggggcacaatggtgcggggaaatctaccaccatatctatgttgacaggaaatttaaaggcaactcgcgggtctatgagcgtggctgggtacgacatgagctcgcaagccgcggccgctcgcgcacacatcggcttgtgtccacaacacaacgtcttgttcaacgaactcaccgtcaaggaacatctccaatttttcgcccgcctcaaaggattcaaaggccaacaattgaaagacgaaattgatactcttatcgctaaattggagttggaagaaaagcgtgattatccttctaaaggtctttctggcggtcagaagcgtcggttatgcgttggtatagctttaagcggggcagctcgtgtcgtgctcctggacgaaccaacctcgggcatggaccctgcatcccgacgagctctctgggaccttttgcaacgggagaagaaaggtcgctcaatgatcctgacgacccactttatgaacgaggcagacattctcggcgacagagtggccattatggcgaacggtcgcctgcagtgcgtgggctccccgtacttcctcaagcgtcactatggcgtcgggtacaccctggtcgttgtcaaggatgatgacttcgacttcgaggaatgctctaagctgattcataaatatgtcccaggcagtattatgaaggaagatcgcggttccgaaatcgcgtatagtctcgacaatgattactctcacactttcgaaaatatgttaaatgatcttgaaaagaatattggcacaattaaactgaaaaattatggcttggttgcaactactctggaagatgtatttatgtctgttggcgcggacctggcacctgtacagtcagagtcggacgacactgccaccacaaccaccgactcctcaatggacgatatactcaaacatgaaatcgattcttctttagaacaattggatagagacgagagcagcgtcaaaggtttcagtctattgtaccagcacgttctagcggtgtggatgaagttggccttagtctggatccgatcttggtggctggtgctattgcagtttgccgcccctgtagtcctgataaacgccacgcttggagtcttgcaatacgttatgtcgttcgcgcctatcattacaagcagggttttagatcttacagaaggttatgttctcaccgaggccctgttgagctacaacggttcttcgtcgacgtcgctcggagctctcgcagcacaagcctatgaaacgatgttcaaaacctccggagtcaacagtatggagctcacgttgattggcagtcgaccagtcgaagattattatctagagagggcaaacgacacagtggcaatggctaacctacgtcaccgcctgctgatcggctccactttcgatgaaaactcagctacggcctggttcagtaactttggctaccacgacgtagccacgtcgctagcaactgtctactcagctatactcaaagccaagaactcaaccgcttttatgaacgtttacaatcatcccctggaagccacgtattcggatcaaagtgacttgcagacgatgatagcgatgttgtccatgcagctggcgtctggcatcggcagcagtgtgggcatcgtgagcgctgtgttcatcatgttctatatcaaggagcgtgtatcgggcgccaagttgctgcagaaggcggcgggcgtgcagccggccgtgctgtggggcgcggccgctgtgttcgactggacgtgcttcttactcacctgcatatctatagtcatctcctgtgcggccttccaggtcataggcctgtctacagcttccgagttgggccgcatgtacctgtgtgcgatggtgtacggcgcggcgatgttgccgttcagctacatcatgtcgcacgtgttcagaggaccagccgtcggcttcgttagcttcttcttcatgaatgtcatctttggtatgatgggcccgcaagtggtggaggcgctgtcttcgccgacgctcaccacgcagcacgtggcgcacattatggacaacgtgctgcagttcttcccgctctacagtcttgttacatcagtcaggtatttgaaccagatcggcctccgtgagtacacgtgcctgcaaagctgtgaatacttgcaggcggtgtaccctaacgtcgagtgcacgatggccagcatgtgcgaattctccagtaactgttgcgttcgagataacccgtacttcgactgggaggagcctggcgttctgcggtacttggtcgccatgacaggcacctgcgccgttctatggacgattctgatggtcatcgagtacagactcttccaaaaggtattaagattccgcaagaccccgccgccagtggacgagagctcgttagacgaggacgtggcgcgcgaggctgagagcgcgcgtcacacgcactacgccgaccgcgcgaaccatgccctcctcgccacggacctcgccaagtactacgggaaacatctcgcggtgggccaagtgtccttcagtgtaagcgacggcgaatgtttcggtctgctgggcgtgaacggcgcgggcaagaccacgaccttcaagatgctgatgggcgacgagtccatctccagcggcgaggcctacgtcagcggacactctgtgcggaagaacctgaacagggtgcatgagaatattggttactgtccgcaattcgacgcgttattcggcgagctgactggtcgcgagacgctccgcatgttcgccctgatgcgaggacttcgcctcagcacggccgcgcccgctgtggagacgctctcacacgcactcggcttcttaagacatctcgataagagggtggatcaatattcaggaggcactaagcgaaagctgaacacagcgatagcgttcttaggaaagacgagacttgtgttcgtcgacgaacctaccaccggcgtagaccctgctgctaaacgacatgtatggcgagccacgcggggcgtgcagcgcgcaggccgtggcgtggtgctgacgtcacacagcatggaggagtgcgaagcgctgtgctcacgactcaccatcatggtcaacggacggttccaatgccttggaacaccacaacatctcaagaataaattctctgaaggttttacgttaactatcaaaatgaaaatggaggataatcctgaaacatcgtcgaacagcagcgcaatcagtaaagtggaccttgtcaaggaatacgtcgaagctaatttccagactcctaggataatggaggaataccaaggtctactaacatactacctaccagaccggacaatggcgtggtcacgaatgttcggtatcatggagcgagcgaaacgagacttagaaatcgaggattacagcatatcacagacgacattagaacagatattcctacaattcacaaagtaccagcgacaagaaggcgatgaatcatag

SEQ ID NO: 19 - protein, 1743 aa, Chrysodeixis includens
MKMRREAKPAGAFMKFRLLMWKNFLQQWRHRLQTVTELLLPVLTMTLVLVLRWQMEPSMVGTLTYPPIPAHTLNYSTAILAGMNLTQMSIAYSPRSPVLDDVVRTGITNLLVANAKDLLPIFENISIPGLPEIELPSIPEDFNSTLIVEFLKSRIKIEAYNNSHDLRGLYIREESTRVVIAGIEFDDKLYDAESLSNNLSFALRFPERPRMNSFFQRGGRTWRTDIVFPLFEMPGPRYPWSWEGGRDPGYVNEMFIALQNAISNELISRATGEDLKKFRVNVQRFPHPPYILDMAVDLLQFMFPMFIMLSFSYTAVNIARAVTVEKELQLKETMRIMGLPTWLHWTAWFCKQFLYLLITAILIIVLLKIHWFTNEEGFSEYAVFTNTPWTVLFFFMVLYLSCAIFFCFMISSFFSKGSTAALCMGVVWFLSYVPAVLLAMDIDMSTAMQVFTCLSINSAMSYGFQLLLAKEAVGGLQWGDFMSSPAAETNRFVFGHVVIMLVVDCVLYMLVTLYLEQVMPGPFGTPKPWYFPFQLKFWFPNYSSDVGFILENEVSESEDIIKEKDPIDHTIGVKMHDLTKIYGNNVAVNHLSLNIYNDQITVLLGHNGAGKSTTISMLTGNLKATRGSMSVAGYDMSSQAAAARAHIGLCPQHNVLFNELTVKEHLQFFARLKGFKGQQLKDEIDTLIAKLELEEKRDYPSKGLSGGQKRRLCVGIALSGAARVVLLDEPTSGMDPASRRALWDLLQREKKGRSMILTTHFMNEADILGDRVAIMANGRLQCVGSPYFLKRHYGVGYTLVVVKDDDFDFEECSKLIHKYVPGSIMKEDRGSEIAYSLDNDYSHTFENMLNDLEKNIGTIKLKNYGLVATTLEDVFMSVGADLAPVQSESDDTATTTTDSSMDDILKHEIDSSLEQLDRDESSVKGFSLLYQHVLAVWMKLALVWIRSWWLVLLQFAAPVVLINATLGVLQYVMSFAPIITSRVLDLTEGYVLTEALLSYNGSSSTSLGALAAQAYETMFKTSGVNSMELTLIGSRPVEDYYLERANDTVAMANLRHRLLIGSTFDENSATAWFSNFGYHDVATSLATVYSAILKAKNSTAFMNVYNHPLEATYSDQSDLQTMIAMLSMQLASGIGSSVGIVSAVFIMFYIKERVSGAKLLQKAAGVQPAVLWGAAAVFDWTCFLLTCISIVISCAAFQVIGLSTASELGRMYLCAMVYGAAMLPFSYIMSHVFRGPAVGFVSFFFMNVIFGMMGPQVVEALSSPTLTTQHVAHIMDNVLQFFPLYSLVTSVRYLNQIGLREYTCLQSCEYLQAVYPNVECTMASMCEFSSNCCVRDNPYFDWEEPGVLRYLVAMTGTCAVLWTILMVIEYRLFQKVLRFRKTPPPVDESSLDEDVAREAESARHTHYADRANHALLATDLAKYYGKHLAVGQVSFSVSDGECFGLLGVNGAGKTTTFKMLMGDESISSGEAYVSGHSVRKNLNRVHENIGYCPQFDALFGELTGRETLRMFALMRGLRLSTAAPAVETLSHALGFLRHLDKRVDQYSGGTKRKLNTAIAFLGKTRLVFVDEPTTGVDPAAKRHVWRATRGVQRAGRGVVLTSHSMEECEALCSRLTIMVNGRFQCLGTPQHLKNKFSEGFTLTIKMKMEDNPETSSNSSAISKVDLVKEYVEANFQTPRIMEEYQGLLTYYLPDRTMAWSRMFGIMERAKRDLEIEDYSISQTTLEQIFLQFTKYQRQEGDES

SEQ ID NO: 20 - DNA, 1932 bp, Artificial Sequence
  shuffled variant
gcgaaagcccagcgttccggtaccagatccatgggaaactctgtccttaattccggtcgtaccacaatatgtgatgcatacaacgtggcagctcacgaccctttctcattccagcacaaatcactagacactgttcagagggaatggactgagtggaagaagaacaatcattcgctatatctcgacccgatcgtcggaaccgtggcttcattcttgctcaagaaggtgggttctctcgttggtaagaggattctctcggaactaaggaatttgatcttcccctccggtagcacaaatctcatgcaggatatactccgtgagaccgagcaattcctgaaccagcgactggacacggacaccttggcacgagttaatgctgaattgacaggtctacaggcaaatgtcgaggagttcaatcgccaagttgacaacttcctaaatcccaatcgtaacgccgtgcctttgtctattacgtcgtccgtcaacacgatgcagcagctattcttgaaccggttacctcaattccagatgcaaggctaccaattgttgttactcccgttattcgcccaagctgctaatcttcacctgagcttcatcagggatgtcatcctgaatgcagacgagtggggcatatcggcagctacactacgtacttatagggattacctgaagaactacacgcgtgactactcaaactactgtatcaacacctatcagtccgccttcaaaggcctgaatacaaggctccacggtacgttggagtttcggacatacatgttcctgaacgtgttcgagtatgtctccatctggtcgcttttcaagtaccagtcattgctggtctcgtcaggtgctaacctatacgcaagtggaagtggacctcagcaaacccaatcgttcacgagtcaagactggccattcctgtatagcttgttccaggtcaactccaactacgtgctgaacggcttctcaggtgctcgattgtccaacactttcccaaacatcggtggacttccaggaagcactacgactcatgccctgctggctgcacgagtcaactactctggtggaatctcaagtggcgatattggagcttcaccattcaaccagaacttcaactgcagcacattcctgcctcctttgcttacgccattcgttagatcctggctcgacagtggaagtgatcgagaaggagtcgctactgtgaccaactggcagacagagagtttcgagacaacactcggtctacgatcaggagcattcacagcaagaggaaacagcaactacttcccagactacttcattcgaaacatctctggagtacctctagtcgttaggaacgaagaccttcgtcgtcctctgcactacaatgagatcaggaacattgcctcaccttcaggtacacctggtggagcacgagcatacatggtctcagttcacaaccgtaagaacaacatccatgcagttcatgagaacggatcaatgatccacttggctccaaacgactacaccggatttacaatcagtcctatccacgccactcaggtgaacaaccagactcgaacgttcatcagtgagaagtttggaaaccaaggcgattctctgaggtttgagcagaacaataccacggcaaggtacactctcaggggtaatggaaactcttacaacctatacttgcgtgtctccagcataggcaattcaactatcagggttaccatcaacggtcgagtgtacacagctacaaacgtcaataccaccactaacaacgatggtgtaaacgacaatggtgctcgcttcagcgacatcaacatcggaaacgtagtcgcaagcagtaacagtgacgtacctctggacattaacgttacgttcaactcaggcacacagttcgatttgatgaacaccatgctggtaccgacaaacattagcccattgtat

SEQ ID NO: 21 - protein, 644 aa, Artificial Sequence
  shuffled variant
AKAQRSGTRSMGNSVLNSGRTTICDAYNVAAHDPFSFQHKSLDTVQREWTEWKKNNHSLYLDPIVGTVASFLLKKVGSLVGKRILSELRNLIFPSGSTNLMQDILRETEQFLNQRLDTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHGTLEFRTYMFLNVFEYVSIWSLFKYQSLLVSSGANLYASGSGPQQTQSFTSQDWPFLYSLFQVNSNYVLNGFSGARLSNTFPNIGGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTFNSGTQFDLMNTMLVPTNISPLY

SEQ ID NO: 22 - DNA, 26 bp, Helicoverpa zea
atgagattagaaacgaggcacgctag

SEQ ID NO: 23 - DNA, 28 bp, Helicoverpa zea
ttataacgtcgttccttcttctctttgg

SEQ ID NO: 24 - DNA, 23 bp, Ostrinia nubilalis
atgaatcggaaaagaggaagcgg

SEQ ID NO: 25 - DNA, 29 bp, Ostrinia nubilalis
ttattgaattgcagcctcttgttgatatt

SEQ ID NO: 26 - DNA, 23 bp, Chrysodeixis includens
atgaagatgaggcgagaggctaa

SEQ ID NO: 27 - DNA, 24 bp, Chrysodeixis includens
ctatgattcatcgccttcttgtcg